(19) United States
(12) Patent Application Publication
(10) Pub. No.: US 2026/0245711 A1
(43) Pub. Date: Aug. 20, 2026

(54) SYSTEMS AND METHODS FOR EQUIPMENT TRACKING AND COMPLIANCE MONITORING WITHIN A FACILITY

(71) Applicant: VSI Health and Hygiene Group, LLC, Alpharetta, GA (US)

(72) Inventor: Charles Agnew Osborne, JR., Cumming, GA (US)

(21) Appl. No.: 19/643,223

(22) Filed: Apr. 9, 2026

Related U.S. Application Data
(63) Continuation-in-part of application No. 19/400,459, filed on Nov. 25, 2025.
(60) Provisional application No. 63/831,430, filed on Jun. 27, 2025.
(60) Provisional application No. 63/725,069, filed on Nov. 26, 2024.

(51) Int. Cl. G16H 40/40; A47K 5/12; A47K 10/32; G01J 5/00; G01S 13/50; G05B 19/418

(52) U.S. Cl. CPC G16H 40/40; A47K 5/1217; A47K 10/32; G01J 5/0025; G01S 13/50; G05B 19/4187; G05B 2219/42337

(57) ABSTRACT

A dispenser includes a dispensing mechanism, at least one activation sensor, a power management system, and a control system configured to activate the dispensing mechanism to dispense a fluid or sheet material in response to a signal from the at least one activation sensor. The power management system can include at least one detection sensor and is configured to connect and disconnect operative components of the dispenser, such as the control system, from a power source when the at least one detection sensor detects the presence of an individual within a prescribed detection range, area, or zone thereof. In addition, some of the operative components, such as the control system and the dispensing mechanism, can remain disconnected from the power source until the at least one activation sensor has been activated.

### CROSS REFERENCE

This application is a Continuation-in-Part of U.S. Non-Provisional Patent Application No. 19/400,459, filed November 25, 2025, and which claims the benefit of U.S. Provisional Application No. 63/725,069, filed November 26, 2024, and claims the benefit of U.S. Provisional Application No. 63/831,430, filed June 27, 2025.

### INCORPORATION BY REFERENCE

U.S. Non-Provisional Patent Application No. 19/400,459, filed November 25, 2025, U.S. Provisional Application No. 63/725,069, filed November 26, 2024, and U.S. Provisional Application No. 63/831,430, filed June 27, 2025, are specifically incorporated by reference herein as if set forth in their entireties.

### TECHNICAL FIELD

The present disclosure generally is directed to systems and methods for control and monitoring various systems and equipment, such as control and monitoring of dispensers within a facility, and to systems and methods for tracking and locating equipment and personnel within the facility and for facilitating determinations of compliance with procedures and/or protocols at the facility. In some aspects, the present disclosure is directed to systems for monitoring, communication with and control of facility equipment such as dispensing systems, assemblies, dispensers, which can be linked to and/or configured to communicate and cooperate with equipment tracking systems for mapping movement of and locating equipment within a facility, as well as with compliance systems for ensuring compliance with procedures and/or protocols, such as compliance with health and safety protocols of a medical facility. Other aspects are also described.

### BACKGROUND

Dispensers for dispensing sheet materials, such as tissue paper, paper towels, and other paper products, as well as for dispensing fluid materials or liquids like soaps, scrubs, and hand sanitizers, are commonly used in hospitals, restrooms, homes, and various other facilities for sanitary and hygiene purposes and to help control the amount of paper or fluids dispensed. Many types of automated dispensers typically are maintained in a substantially active state, continuously looking for the next user and ready to dispense an amount of paper or fluid. However, by remaining in a substantially always active or powered-on state, the steady state power consumption of such dispensers can remain at a generally constant high level, which can lead to increased operational costs and in cases where the dispensers are powered by batteries, appreciably shorter battery life.

For example, facilities such as hospitals have strict health and safety protocols that require personnel to diligently wash their hands and use hand sanitizer, etc.., and thus such dispensers are often left in a fully powered-on, active and ready to dispense state, leading to increased power consumption. In addition, tracking the actual use of the dispensers by personnel, and thus their compliance with the facility’s health and safety protocols, can be difficult. While systems have been developed to help monitor compliance with such protocols, a need still exists for improving accuracy and efficiency of such monitoring systems, as well as for enabling enhanced tracking and location of personnel and equipment moving about a facility, such as when needed for responding to emergency situations.

Therefore, it can be seen that there is a need for dispensing systems, assemblies, dispensers, and methods of operation thereof that can provide increased control of the operation therefore to minimize power consumption without disrupting use of the dispensing systems, which can provide for enhanced monitoring, collection and communication of information regarding the use and status of the dispensing systems, assemblies and dispensers, and which also can assist in tracking compliance and mapping and location of personnel and equipment moving about a facility. The present disclosure addresses these and other related and unrelated issues in the art.

### SUMMARY

In one aspect, the present disclosure is directed to systems and methods for the operation/control of, monitoring and tracking use of, movement and/or location of equipment and personnel within a facility. In embodiments, such equipment can include dispensing systems and/or assemblies, including dispensers and methods of operation and assembly thereof, and in some embodiments, further can include other types of equipment that can be moved about the facility.

In some example embodiments, such systems can include equipment tracking systems and compliance systems that can interface with a plurality of dispensing systems positioned at selected, identified locations about the facility. In embodiments, such dispensing systems can include at least one dispenser or dispensing assembly, and in some embodiments, can include a series of dispensers or dispensing assemblies that can be in communication with one another. In embodiments, each dispenser of the dispensing systems generally can be operable for dispensing selected amounts of a sheet material, for example, paper products, including paper towels, tissue, napkins, etc., and/or for dispensing fluid materials or liquids, e.g., liquid soap, hand sanitizers, gels, etc.

In addition, in various embodiments, the dispensing systems can be used in a variety of different environments, including, without limitation, use in commercial facilities (e.g., in operating, patient and treatment rooms, bathrooms and/or kitchens of restaurants, large venues such as arenas, airports, schools, etc..) and other locations; and in some embodiments, also can be used in a residential setting (e.g., in a bathroom in a person’s home). In embodiments, each dispenser of the dispensing systems can include a dispenser housing having a dispensing mechanism and a supply of a fluid or a sheet material to be dispensed, which can be dispensed in metered, predetermined amounts during each dispensing operation or cycle. In embodiments, the housing can be a metal, plastic, or other, similar rigid material, and in some embodiments, can be treated with an antimicrobial agent.

In an example construction, the dispenser (or dispensers in embodiments where the dispensing systems include a series of dispensers) can comprise a sheet material dispenser having one or more rolls of a sheet material (e.g., tissue, paper towels, etc..), a feed roller that can be driven manually or by a motor to feed a predetermined amount of sheet material during a dispensing operation, and, in embodiments, one or more pressing rollers configured to urge the sheet material against the feed roller such that the sheet material is pulled or drawn therebetween during a dispensing operation. In another construction, the dispenser can include a liquid dispenser with a supply chamber or reservoir containing a fluid, e.g., liquid soap, hand sanitizer, etc., one or more nozzles or other discharge outlets for dispensing the liquid to users (e.g., upon activation of one or more sensors or other activation mechanisms), and a pumping mechanism for directing the fluid from the supply chamber to a discharge outlet(s).

In embodiments, the dispenser, or, in embodiments such as where the dispensing system includes a series of dispensers, each dispenser of the dispensing system further can include a power supply and a power management system in communication with a dispensing assembly. In embodiments, the dispensing assembly can include a dispensing mechanism, at least one activation sensor, and a control system.

In embodiments, the dispensing mechanism can include a feed roller that can be driven by a drive assembly that, in embodiments, can include a motor or other actuator, or can be manually operable to drive the rotation of the feed roller, and one or more pressing rollers. The dispensing mechanism can communicate with the control system and/or the at least one activation sensor and generally will be configured to dispense a selected amount of a fluid or sheet material in response to an activation or initiation signal.

The at least one activation sensor is configured to detect and be activated by a user to initiate dispensing of the selected amount of the fluid or sheet material. For example, when a user’s hand is within a close enough proximity to the activation sensor, or, in some embodiments, when the user engages the activation sensor such as by placing their hand within a detection zone for the activation sensor or making some other activation gesture or action, the activation sensor can send an activation signal or prompt to the control system to initiate a dispensing operation. In embodiments, the detection zone can be located within a recessed area or pocket.

In embodiments, the control system will be in communication with a power source and with various components of the dispenser, such as the dispensing mechanism, the at least one activation sensor. In addition, in embodiments, where one or more dispensers of the dispensing system include a power management system in communication with the control system, the power management systems thereof can be configured to control the powering on/off of the control system and the other components of the dispenser. In embodiments, the control system also can be in communication with and/or control other components such as a display, communications system, and other features of the dispenser.

In addition, in some embodiments, the dispenser can include one or more monitoring sensors configured to monitor the dispensing system, usage of the dispenser, the occurrence of fault conditions such as low power, low or no supply, transmissions from identifying badges carried by individuals, etc. For example, in some embodiments, the dispenser can include a detector configured to monitor/measure a level or amount of a sheet material or fluid remaining in a supply and communicate this to the control system. In embodiments, if the level is determined to be below a threshold level or amount, the control system can generate an alert or otherwise signal a low/no supply condition to facilitate replacement of the supply when needed (so as to not waste unused fluid or sheet material within the supply that can occur with premature replacement of the supply) and without undue delay.

Still further in embodiments, the dispenser can include a display that can include indicators such as a screen or lights, and a communication system. In embodiments, the communication system can include a transmitter that can be linked to or included as part of the control system (or, in embodiments, multiple transmitters linked to various components or assemblies of the dispenser). The transmitter generally will be configured to transmit information/data such as dispenser usage and diagnostic information, tracking information regarding individuals’ use of the dispenser (or no use), fault conditions such as a low power or low/no supply of material (e.g., paper or fluid material), or other information, to a server, network, mobile application, facility control system, mobile or wireless device, or combinations thereof.

In embodiments, the power management system is in communication with the power supply and the operative components of the dispenser, such as the controller of the control system, dispensing assembly, including, for example, the dispensing mechanism (e.g., motors, sensors, etc.), the at least one activation sensor, and/or other components. Additionally, the power management system will include at least one detection sensor, such as, in one embodiment, at least one passive infrared radiation (PIR) sensor. The at least PIR sensor generally will be configured to capture infrared radiation of one or more individuals within a prescribed detection range, area, or zone, and upon such detection, can initiate a powering-on of the controller, at least one activation sensor and/or other components, including, in some embodiments, one or more receivers, a transmitter, etc. (e.g., couple or connect the controller, at least one activation sensor, and/or other components to a power source such as a battery).

Alternatively, or in addition, in some further embodiments, at least one detection sensor of the power management system can comprise a radar sensor configured to at least periodically emit pulses of radio waves. In such an embodiment, radar sensor can activate/power on various operative components of the dispenser based on a timing of a return of reflected radio waves can indicate whether an individual has passed within the detection range, area, or zone. In embodiments such as in warmer and/or more well-lit environments where temperatures are closer to a body temperature, the use of a radar sensor may be operable to detect the presence of individuals within the detection range, area, or zone with increased accuracy and further can, in some embodiments, enable a reduction in the size of the detection range, area, or zone of the power management system.

In embodiments, the control system, or at least various components thereof, such as, for example, the controller, one or more sensors, the transmitter, etc.., and the dispensing mechanism, can be kept in a powered down state (e.g., disconnected from the power supply) if the presence of a user within the prescribed detection range, area, or zone is not detected by the at least one PIR sensor, and can be reconnected to the power supply or otherwise placed in a powered-on state when the presence of one or more individuals is detected within the prescribed detection range, area, or zone For example, the power management system can include at least one PIR sensor and an associated controller (e.g., the at least one PIR sensor can have its own controller) that can include programming to place the control system in a powered down state (e.g., after a prescribed time, or other event).

In embodiments, the power management system can include or can be in communication with a switch or other connection mechanism configured to disconnect or decouple one or more of the operative components of the dispenser, such as, for example, the dispensing assembly, including the control system, activation sensor, dispensing mechanism, and other components, from the power source, such that the primary operative components of the dispenser, with the exception of one or more PIR sensors, are not drawing power when not in operation. When a user enters the detection area or zone covered by the at least one PIR sensor, the power management system can engage the switch to enable power from the power source to be supplied to one or more components of the dispensing assembly.

For example, in embodiments, when infrared radiation from the user is detected by the at least one PIR sensor, the power management system can initially enable the connection of the activation sensor(s) to the power source, while maintaining other components such as the dispensing mechanism and the control system of the dispenser to remain disconnected from the power supply and/or otherwise remain in a substantially powered down state. The control system, dispensing mechanism and other components can remain in their powered-down/off state, drawing minimal to no power from the power source, until the activation sensor is triggered, such as by detection of motion by an individual or user. In embodiments, if the activation sensor is not triggered/activated (e.g., no activation signal is generated within a preset time), the power management system can shut down power to the activation sensor, without the remaining components for the dispensing assembly and the dispensing system overall having to draw power.

If the activation sensor is engaged by the user, one or more activation signals or prompts can be sent, either directly to the control system, or initially to the power management system, to cause the controller of the control system and the dispensing mechanism to be connected to the power supply and/or otherwise be placed in a powered-on state. Once in the powered-on state, the controller is configured to activate the dispensing mechanism to dispense the selected amount of the fluid or sheet material upon receipt of the one or more activation signals or prompts from the at least one activation sensor.

Thereafter, the power management system can disconnect the control system, the at least one activation sensor, and the dispensing mechanism from the power source and connect the at least one activation sensor to the power source when the at least one PIR sensor does not capture infrared radiation of one or more individuals within the prescribed detection range, area, or zone. For example, after a prescribed period of time with no detection of infrared radiation by the PIR sensor, the operative components of the dispenser can be disconnected from the power supply. In addition, in embodiments, the control system and the dispensing mechanism, as well as other operative components such as a transmitter and display, etc. can remain disconnected from the power source until the at least one activation sensor has detected the user.

In various embodiments, the at least one PIR sensor of the power management system, the control system (e.g., a controller of the dispenser), the at least one activation sensor, and the dispensing mechanism, as well as other operative components of the dispenser/dispensing system, can include a smart architecture, with each having their own associated processor(s) or CPU, which can be configured and/or include programing or instructions enabling each of such operative components to perform its functions independently of the others. In addition, in embodiments, the at least one PIR sensor of the power management system, the control system, at least one activation sensor, and the dispensing mechanism, and/or other operative components of the dispenser/dispensing system, can comprise changeable or “plug-and-play” components, enabling such components, and, in embodiments, the processors thereof, to be removed and replaced and/or adapted to update the dispenser, such as to accommodate different or changing usage conditions.

In another aspect, the dispensing system can comprise a series of dispensers arranged throughout out a selected area (e.g., a bathroom such as in an airport, etc..). In embodiments, each of the dispensers can be independently operable. In some embodiments, the series of dispensers can include a lead dispenser and a plurality of drone dispensers. The lead dispenser can include a power management system and a control system that can be configured for controlling one or more operations of the lead dispenser, and at least partially control operation of one or more of the drone or other dispensers. For example, the lead dispenser can be positioned at a central or initial location selected to detect the user initially entering into the detection area space, or zone, and when it detects the user, can in turn wake up or otherwise cause the power management systems of the one or more drone or other dispensers to be placed in a powered-on or active state connected to a power supply, while the dispensing assemblies of such dispensers remain in a substantially powered-off state, for example, being disconnected from the power supply. Thereafter, each drone or other dispenser can be operated independently to feed a fluid or sheet material only when activated, while the dispensing assemblies thereof otherwise remain in the substantially powered-off state.

In embodiments, the dispensing system can also include, be linked to, or can be part of a network through which various dispensers, for example, one or more drone dispensers and a lead dispenser within an area or facility, can communicate with each other and with a central server, facility management system, to the cloud, or other recipient (e.g., via Wi-Fi or other network connection). In embodiments, such communications generally will be provided wirelessly.

For example, in embodiments, where the dispensing system includes a central, primary or lead dispenser and one or more secondary or drone dispensers, the lead dispenser can be configured to communicate information related to the lead dispenser and/or information received from the plurality of drone dispensers to other recipients, such as to a central server, facility management or building control system, manager, to the cloud, etc... via the network. In some embodiments, the primary or lead dispenser, or each individual dispenser, including the primary or lead dispenser and secondary or drone dispensers, can be configured to communicate information such as alerts for low supplies of sheet materials or fluids, jams, low power, or various other operating conditions thereof, to a central server, facility management or building control system, manager, to the cloud, etc.

In embodiments, the dispensing system, including each dispenser thereof, also can include one or more transmitters configured to transmit and/or receive signals for exchanging information and transmitting data (e.g., dispenser usage, supply levels, etc..), diagnostic information including alerts, and/or other notifications from one or more dispensers to the lead dispenser (e.g., if the one or more drone dispensers are experiencing an error condition, a low power condition, and/or a low supply condition), and/or to other recipients. In embodiments, some of the dispensers can be configured to send messages to a lead or primary receiving dispenser, which can then send an information packet including data/information collected/received from one or more dispensers to the central server, facility management or building control system, manager, to the cloud, etc., (e.g., wirelessly such as by Bluetooth® or Wi-Fi); while in other embodiments, each dispenser can individually transmit its information, in addition to or without sending such information to a lead or primary receiving dispenser.

In embodiments, the dispensers can be configured to send messages such as alerts, dispenser operation and supply information, refill reminders, etc.., at predetermined times. For example, in some embodiments, the dispensers can be configured to transmit messages such as alerts and other information to the central server, facility management or building control system, manager, to the cloud, etc. when the dispenser is activated. Thus, when the dispenser is in a powered down state in which its controller is powered off, no messages may be sent, conserving power since the dispenser can remain disconnected from Wi-Fi until a dispensing operation is initiated, has been completed, and/or is in progress. In addition, once the message has been sent, the network connection (e.g., Wi-Fi) of the dispenser can be closed, for example, when the power management system disconnects the control system, the network connection also can be disconnected, including powering down or off the transmitter and/or receiver.

In still other still other embodiments, if, for example, an alert indicating an error or fault condition such as a jam, low or no supply condition, has been generated, the dispenser can further be configured to connect to the network and transmit an update to indicate if/when the fault condition has been cleared.

In addition, in various embodiments, the control system of the dispenser can include one or more transmitters and/or receivers configured to at least periodically monitor the selected activation area or zone within a determined proximity of the dispenser to detect signals being transmitted by transmitters carried by individuals. For example, the dispenser can be part of a dispensing system located within a hospital or other, similar facility in which compliance with certain health and safety protocols (e.g., requirements for doctors, nurses and other personnel to perform handwashing and other sanitizing operations upon entering and/or leaving a patient’s room). In some such instances, the dispensing system can communicate with or be integrated with or as a part of a compliance system for the facility.

In embodiments, the control system of one or more dispensers of an overall dispenser system can include one or more processors or controllers (which also can be referred to as CPUs) and further can be configured to provide bidirectional communications. The bidirectional communication functionality/operation of the dispensers enables the dispensers to communicate with the badges carried by individuals as part of the compliance system and with the tracking systems associated with identified pieces of equipment for tracking the movements of individuals and the pieces of equipment via use of the dispensers of an overall dispenser system or network of dispensers, without necessitating the use of additional sensors or monitoring systems to be positioned about the facility.

In embodiments, the control system can be configured to look for and/or request identification signals from an individual’s badge at multiple points/times within a dispensing operation. For example, when the control system of the dispenser initially receives a first signal from the individual’s badge, it can identify the presence of that particular individual and proximity to the dispenser. The control system can then search for a second identification signal in conjunction with the activation of the dispensing mechanism, and search for an additional identification signal from the individual’s badge (e.g., a third signal) upon completion of the dispensing operation. If the additional (e.g. one or more second and/or third signals) identification signals are not received and/or a dispensing operation is not initiated after the first identification signal from the individual’s badge is detected, the control system can generate a noncompliance record indicating that the individual failed to use the dispenser. In embodiments, such a noncompliance record can be transmitted together with the identification of the individual associated with a detected badge to server, network, mobile application, mobile or other wireless device, facility control, or other system, for tracking and monitoring of movements of individuals throughout the facility and their compliance with health and safety protocols.

Still further, in embodiments, the power management system of one or more of the dispensers can be configured to selectively power up the dispenser periodically (e.g., at preset time intervals) to enable transmission of messages such as alerts, status messages, etc. at selected time intervals. For example, in embodiments, the power management system (e.g., at least the PIR sensor) can include a processor or CPU that, in addition to programming/instructions for controlling the connection of the operative elements of the dispenser to the power supply, can include programming/instructions to turn on the transmitter and control system of the dispenser, with or without powering on the activation sensor(s), dispensing mechanism or other components, at least for a time sufficient to send the message. Thereafter, in embodiments, the power management system can power down (e.g., disconnect from the power supply) the control system and transmitter, such as, for example, upon receiving a confirmation from the recipient of the message (e.g., a central server, facility management or building control system, manager, to the cloud, etc..) that the message has been received, or after a selected time period, which can allow for additional communications between the dispensers and the recipient.

In some embodiments, the power management system can include a series of PIR sensors that can be configured to connect different components and/or multiple dispensers of a dispensing system to power. For example, in embodiments, each dispenser of the dispensing system can include a series of PIR sensors, which further can each include a controller. Each PIR sensor can be configured to selectively connect a controller (e.g., a processor or CPU) configured for controlling operation of each of one or more different components, and/or an overall dispenser control system controller, to power or place such controllers in a powered-on state. In embodiments, each of the component controllers can be configured to communicate with the PIR controller and with one or more of the component controllers.

For example, in embodiments such as where the dispensing systems include a series of dispensers, multiple dispensers could be linked to a primary or initial power management system (e.g., of a primary or lead dispenser) that includes at least one PIR sensor that can communicate with one or more controllers of a subset of the dispensers (e.g. two or more paper towel dispensers and fluid dispensers for at least some of a series of sinks) in a selected area or location. The controllers in communication with the at least one PIR sensor of such a primary of initial power management system (e.g., for a lead or primary dispenser within an area) could be a main dispenser controller configured to control substantially all operations of the dispenser, or, in some embodiments, can comprise a separate, dedicated processor or CPU configured to communicate with the at least one PIR sensor.

By way of example, in embodiments, when at least one PIR sensor of the primary power management system detects infrared radiation from an individual (e.g., as they enter a room and move near an initial or lead dispenser), it can communicate with a corresponding controller for at least one PIR sensor of a power management system for each dispenser of the subset of dispensers (e.g., soap and sheet material dispensers at series of sinks) to place the at least one PIR sensor of such dispensers into a powered-on state, connected to power, while other dispensers (e.g., sanitizer dispensers and/or tissue dispensers in a stall) can remain in a powered-off state. The remaining components of the dispensers and/or other dispensers (e.g., those in a bathroom stall) can be maintained in a powered-off/powered-down state until the at least one associated PIR sensor (which has been signaled to transition to a powered-on state to look for infrared radiation) for each of those dispensers detects infrared radiation from someone coming within a predetermined detection area, zone, range or distance of the PIR sensor. Thus, in embodiments, additional reductions in power consumption and load shedding among various components of each dispenser of a dispensing system can be provided by limiting the number of active components/dispensers to those in use or likely to be used, while allowing unneeded dispensers and/or components to be shut down and placed into a substantially powered-off state.

In still other embodiments, if, for example, an alert indicating a fault condition such as a low supply condition, has been generated, the dispensers can further be configured to connect to the network and transmit an update to indicate if/when the fault condition has been cleared.

According to some aspects of the disclosure, a dispensing system is provided, comprising: a material; a dispensing mechanism configured to dispense a selected amount of the material during a dispensing operation; a power supply for supplying power to drive operation of the dispensing mechanism during a dispensing operation; and a control system comprising: a controller in communication with the dispensing mechanism and the power supply; at least one activation sensor in communication with the controller and configured to send an activation signal or prompt to the controller to initiate the dispensing operation; and a power management system including at least detection sensor in communication with the control system and configured to capture infrared radiation; wherein at least the controller, at least one activation sensor, and the at least one detection sensor each include a processor; and wherein the at least one detection sensor is configured to connect the controller, the at least one activation sensor, and/or any other operative components of the dispenser, or combinations thereof, to the power source when the at least one detection sensor detects a presence of one or more individuals within the prescribed detection range, area, or zone.

In embodiments, the dispensing system can comprise one or more dispensers configured for dispensing sheet materials.

In other embodiments, the dispensing system can comprise one or more dispensers configured to dispense a fluid or liquid material.

In embodiments, the dispensing system further comprises at least one transmitter.

In embodiments, the at least one transmitter is configured to transmit dispenser status information to a server, mobile application, one or more additional dispensers, a facility control system, network, or combinations thereof.

In embodiments, the dispenser status information can comprise dispenser diagnostic information, dispenser usage information, or combinations thereof. In some embodiments, the dispenser diagnostic information can comprise material supply information, power level information, power usage information, or combinations thereof.

In embodiments, the dispensing system can further comprise at least one supply sensor positioned within a housing of the dispenser to monitor and communicate a remaining amount of material of the supply of material to the controller.

In embodiments, the at least one detection sensor of the power management system can comprise a passive infrared radiation (PIR) sensor configured to detect infrared radiation of the one or more individuals within the prescribed detection range, area, or zone.

In other embodiments, the at least one detection sensor of the power management system can comprise a radar sensor configured to detect movement of the one or more individuals within the prescribed detection range, area, or zone.

In some embodiments, the control system includes at least one transmitter linked to the controller, and wherein the controller is configured to engage the transmitter to send an alert upon detection of at least one fault condition. In embodiments, the at least one fault condition can comprise a low or no supply condition, a low power condition, a dispenser jam condition, failure of any of the operative components of the dispenser, or combinations thereof.

In embodiments of the dispensing system, the control system can be further configured to capture information related to movements and/or activities of individuals within a facility to facilitate identifying, logging, mapping, and/or tracking of the movements and/or activities of the individuals throughout the facility.

In embodiments, the dispensing system can further comprise a plurality of badges carried by a plurality of individuals, each badge of the plurality of badges being configured to communicate with the control system to provide information identifying individuals moving within a selected zone, area, proximity or range of the dispensing system.

In some embodiments of the dispensing system, each badge can be configured to transmit a plurality of signals, each of the signals being transmitted at a predetermined distance or at a predetermined signal strength, and wherein the control system can be configured to determine a plurality of positions or movements of each individual within the selected target range based on the distance or signal strength of the signals received from a badge carried by the individual.

In embodiments of the dispensing system, the dispensing system can be located within a medical facility, the medical facility including a facility control system having at least one database configured to store information related to patients and the individuals moving throughout the medical facility.

In various embodiments of the dispensing system, the control system is configured to transmit dispenser use information and identifying information for individuals moving within a selected zone, area, proximity or range of the dispensing system to the facility control system. In embodiments, the facility control system can be configured to cross-reference the dispenser use information with the information stored in the database to facilitate a determination of use of the dispensing system by the patients, the individuals, or a combination thereof.

According to other aspects, a method is provided, comprising: sensing, by an activation sensor, an individual moving within a selected activation zone for a dispenser, the dispenser configured to dispense a selected amount of a fluid or a sheet material; in response to an activation signal from the activation sensor of the at least one dispenser, initiating a dispensing operation, wherein the dispensing operation comprises: activating a dispensing mechanism and dispensing a selected amount of the fluid or sheet material from a supply; wherein prior to sensing the individual by the at least one activation sensor, at least a control system of the dispenser, the at least one activation sensor, and the dispensing mechanism are disconnected from a power source via operation of a power management system connected to the power source; and connecting the at least one activation sensor to the power supply when the power management system detects one or more individuals within the prescribed detection range, area, or zone; and wherein the control system and the dispensing mechanism remain disconnected from the power source until the at least one activation sensor has detected the user within the selected activation zone.

In embodiments, the power management system can include at least one detection sensor.

In some embodiments, the at least one detection sensor of the power management system comprises a PIR sensor; and wherein the at least one activation sensor is connected to the power supply when the PIR sensor detects infrared radiation of one or more individuals within the prescribed detection range, area, or zone.

In other embodiments, the at least one detection sensor of the power management system comprises a radar sensor; and wherein the at least one activation sensor is connected to the power supply when the radar sensor detects movement of one or more individuals within the prescribed detection range, area, or zone.

In embodiments, the method further can comprise monitoring a level or amount of a fluid or sheet material within the at least one dispenser; and if the level or amount of a fluid or sheet material is below a threshold level, and transmitting an alert to a server, network, mobile application, facility control system, mobile or other wireless device, or combinations thereof.

In embodiments, the method further can comprise monitoring usage of the at least one dispenser by one or more of a plurality of individuals moving within a threshold activation proximity, range, or area of the at least one dispenser.

In some embodiments of the method, monitoring usage of the at least one dispenser by the one or more individuals comprises receiving signals from at least one transmitter carried by the one or more individuals by the control system to detect initiation of a dispensing operation.

In further embodiments of the method, monitoring usage of the at least one dispenser can comprise checking for signals from the at least one transmitter carried by at least one individual within the activation proximity, range, or area of the at least one dispenser at more multiple times during the dispensing operation.

In embodiments, the signals include information identifying the at least one individual carrying the at least one transmitter.

In some embodiments of the method, monitoring usage of the at least one dispenser by the one or more individuals comprises receiving at least one signal from the at least one transmitter of the at least one individual prior to activation of the dispensing mechanism, at least a second signal from the at least one transmitter of the at least one individual after the activation of the dispensing mechanism and during the dispensing operation, and at least one signal from the at least one transmitter of the at least one individual after completion of the dispensing operation.

In embodiments, the method can further comprise transmitting the dispenser use information of the at least one dispenser by the one or more individuals to a server, network, mobile application, facility control system, mobile device, or combinations thereof.

In embodiments, the method can further comprise recording a date and time of use of the at least one dispenser by each of the one or more individuals.

In other embodiments, the method can further comprise transmitting one or more signals from a plurality of badges carried by a plurality of individuals throughout the facility; and receiving the one or more signals by the dispensing system to facilitate identifying, mapping, and/or tracking of movements and/or activities of the individuals moving throughout the facility.

In some embodiments of the method, the dispensing system can include a plurality of dispensers; and wherein identifying, mapping, and/or tracking of movements and/or activities of the individuals moving throughout the facility comprises: transmitting one or more signals from a plurality of badges carried by the individuals throughout the facility; and receiving the one or more signals at a series of dispensers of the plurality of dispensers to facilitate the identifying, mapping, and/or tracking of movements and/or activities of the individuals throughout the facility.

In additional embodiments, the method further can comprise cross-referencing information related to the individuals stored in a database with information related to identified, mapped, and/or tracked movements and/or activities of the individuals.

In embodiments, the method can further comprise transmitting dispenser information from a set of drone dispensers of the plurality of dispensers to a lead dispenser of the plurality of dispensers.

In some embodiments, the method can further comprise connecting a power source to a controller, at least one activation sensor, transmitter, one or more receivers, or combinations thereof, of at least the lead dispenser when a detection sensor of any of the drone dispensers detects one or more individuals within a prescribed detection range, area, or zone covered thereby.

In embodiments, the method can further comprise connecting the at least one activation sensor, the controller, transmitter, one or more receivers, or a combination thereof, of at least the lead dispenser to the power supply when a radar sensor of the power management system detects movement of one or more individuals within the prescribed detection range, area, or zone.

In embodiments, the method can further comprise connecting the at least one activation sensor, the controller, transmitter, one or more receivers, or a combination thereof, of at least the lead dispenser to the power supply when a PIR sensor of the power management system detects infrared radiation of one or more individuals within the prescribed detection range, area, or zone.

According to another aspect, a dispenser is provided, comprising: a housing; at least one supply of a material to be dispensed received within the housing; a dispensing mechanism configured to dispense a selected amount of the material; a control system comprising: a controller in communication with the pump and the power supply; at least one activation sensor in communication with the controller and configured to send an activation signal to the controller to initiate the dispensing operation; and a transmitter in communication with the controller and configured to transmit dispenser use information for one or more individuals passing within a selected target range, area, or zone of the dispenser; and wherein the control system is configured to receive a series of signals from at least one transmitter carried by each of the one or more individuals and determine use of the dispenser by each individual passing within the selected target range, area, or zone of the dispenser.

In embodiments, the controller can be configured and/or can include programming configured to look for an/or receive signals from the at least one transmitter of the individual within the selected target range, area, or zone of the dispenser at multiple selected intervals during a dispensing operation.

For example, in embodiments, the controller can look for and/or receive one or more signals identifying each individual from the at least one transmitter carried by each individual within the selected target range, area, or zone of the dispenser prior to initiation or at the start of a dispensing operation, at least once during the dispensing operation, and upon completion of the dispensing operation.

In embodiments, a failure of the controller to receive the signals identifying an individual within the selected target range, area, or zone of the dispenser at each of the selected intervals can indicate a non-use of the dispenser by the individual.

In embodiments, the control system further can be configured to transmit to dispenser use information identifying use or non-use of each identified individual passing within the selected target range, area, or zone of the dispenser to a remote server, facility control system, mobile or other wireless device, database, or combinations thereof.

In embodiments, the dispensing system can be located within a medical facility, the medical facility including a facility control system having at least one database configured to store information related to patients and the individuals moving throughout the medical facility.

In embodiments, the control system can be configured to transmit dispenser use information identifying individuals moving within a selected zone, area, proximity or range of the dispensing system to the facility control system. In some embodiments, the facility control system can be configured to cross-reference the dispenser use information with the information stored in the database to facilitate a determination of use of the dispensing system by the patients, the individuals, or a combination thereof.

In embodiments, the control system further comprises a power management system including at least one detection sensor in communication with the control system and configured to capture infrared radiation; and wherein the at least one detection sensor is configured to connect the controller, the at least one activation sensor, the at least one supply level detection sensor, the pump, or combinations thereof, to the power source when the at least one detection sensor detects one or more individuals within the prescribed detection range, area, or zone.

In embodiments, the at least one detection sensor of the power management system can comprise a PIR sensor configured to detect infrared radiation of one or more individuals within the prescribed detection range, area, or zone.

In other embodiments, the at least one detection sensor of the power management system can comprise a radar sensor configured to detect movement of the one or more individuals within the prescribed detection range, area, or zone.

According to another aspect, a dispensing system comprises: at least one dispenser comprising: a power source; a dispensing mechanism in communication with the power source; a control system in communication with the power source and the dispensing mechanism, and including programming to control the dispensing mechanism for dispensing a fluid or a sheet material; wherein the control system comprises: a controller; at least one activation sensor configured to transmit an activation signal to the controller upon detection of an individual within a selected activation zone, proximity or rage of the dispenser; and a power management system including at least one detection sensor configured to detect a presence of one or more individuals; wherein the power management system is configured to connect at least the at least one activation sensor to the power source when the at least one detection sensor detects the presence of one or more individuals within a prescribed detection range, area, or zone; and wherein the controller and the dispensing mechanism remain disconnected from the power source until the at least one activation sensor has transmitted the activation signal to the controller to initiate a dispensing operation.

In embodiments, the dispensing system can comprise one or more dispensers configured for dispensing sheet materials; while in other embodiments, the dispensing system can include at least one dispenser configured to dispense a fluid material.

In other embodiments, the at least one detection sensor of the power management system can comprise a radar sensor configured to detect movement of the one or more individuals within the prescribed detection range, area, or zone.

In other embodiments, the at least one detection sensor of the power management system can comprise a PIR sensor configured to detect infrared radiation of the one or more individuals within the prescribed detection range, area, or zone.

According to a further aspect, a method comprises: detecting a presence of one or more individuals within a prescribed detection range, area, or zone of a dispenser; wherein the at least one dispenser is configured to dispense a selected amount of a fluid or sheet material; connecting at least one activation sensor to a power supply to place the at least one activation sensor in a powered-on state; if the at least one activation sensor detects an individual within a selected activation zone of at least one dispenser: connecting a controller of the dispenser to the power supply to place the controller in a powered-on state; connecting a dispensing mechanism of the dispenser to the power supply to place the dispensing mechanism in a powered-on state and initiating a dispensing operation; wherein the dispensing operation comprises: activating a dispensing mechanism; drawing a fluid or a sheet material from a supply; and discharging the fluid or sheet material.

In embodiments, detecting the presence of the one or more individuals comprises detecting movement of the one or more individuals within the prescribed detection range, area, or zone with a radar sensor.

In other embodiments, detecting the presence of the one or more individuals comprises detecting infrared radiation of the one or more individuals within the prescribed detection range, area, or zone with a PIR sensor.

According to yet another aspect, a dispenser is provided, comprising: at least one supply of a material to be dispensed; a dispensing mechanism configured to dispense a selected amount of the material; and a control system comprising: a controller in communication with the dispensing mechanism and configured to initiate a dispensing operation and control operation of the dispensing mechanism to dispense the selected amount of material; at least one activation sensor in communication with the controller and configured to send an activation prompt to the controller to initiate the dispensing operation; and a power management system in communication with a power supply is configured to detect one or more individuals within a prescribed detection range, area, or zone and in response, connect the at least one activation sensor to the power supply while the controller and dispensing mechanism remain disconnected from power; and a transmitter configured to transmit dispenser use information.

In embodiments, the power management system comprises at least one detection sensor of the power management system including a radar sensor configured to detect movement of the one or more individuals within the prescribed detection range, area, or zone, a PIR sensor configured to detect infrared radiation of the one or more individuals within the prescribed detection range, area, or zone, or a combination thereof.

In embodiments, the dispenser is part of a dispensing system. In some embodiments, the dispensing system includes a plurality of dispensers. In further embodiments, the dispensers can include fluid dispensers, sheet material dispensers, or combinations thereof.

In addition, according to some aspects, a system is provided and can comprise equipment and/or personnel tracking systems, one or more compliance systems, and a dispensing system or plurality of dispensing systems that can be linked in communication with each other and with a facility server, facility control, cloud server, network, mobile application, or combinations thereof. The tracking systems, compliance system(s), and at least a plurality of the dispensers of the dispensing system(s) within a facility can operate together, as part of an integrated system for monitoring, tracking and generation of information to identify movements and locations of personnel and equipment throughout/within a facility, such as a hospital or other medical facility, lab, etc., The equipment and/or personnel tracking systems and compliance system(s) of the facility can cooperatively communicate with a plurality of dispensers of the and dispensing system(s), which in turn can communicate with a facility control system, a server for the facility or a cloud server, or other information processing and/or storage system for creation of records of movement of identifiable equipment and personnel about the facility and usage of dispensers by personnel, to enable monitoring and tracking of movements and location of equipment and personnel about the facility, enable monitoring and tracking of compliance with dispenser usage protocols or procedures by such personnel; and further enable additional dispenser usage and diagnostic and/or status information to be communicated so that the status/conditions of the dispenser, such as battery/power levels, supply levels, fault conditions and other information, can be transmitted to and accessed by the facility control system and, in embodiments, transmitted to and/or accessed by a mobile device such as a tablet, phone, etc.

Such information can be collected and transmitted by dispensers that are generally required within such facilities, without requiring use of additional monitoring devices and transmitters to be used. As a further result, such an integrated system for the monitoring and tracking movements and locations of equipment and personnel, monitoring compliance and dispenser usage and diagnostics, and for management of operations, including power usage and reporting of dispenser usage and diagnostic and other operating conditions can be installed as a new or retrofit system.

In embodiments, the tracking systems can be linked to an associated piece of equipment that can be movable about a facility. In embodiments, each tracking system will send tracking signals that can include an identification signal or signature that can be used to identify the piece of equipment associated with that tracking system. In addition, in an example embodiment, each of the tracking systems can include a tag, badge or similar device that can be secured to the piece of equipment, and which can comprise a sensor, detector and/or a controller configured to detect motion of the piece of equipment and in response to this movement, activate a transmitter to cause the tracking signals to be transmitted. In embodiments, the compliance system can include badges, tags, or similar devices that each include one or more transmitters that transmit signals including identification information identifying the badge and/or an individual carrying the badge.

In some embodiments, the tracking signals can be sent on a substantially continuous basis while the piece of equipment is moving. Alternatively, the tracking signals can be sent in periodic pulses during movement of the piece of equipment. When the piece of equipment stops moving, the tracking system can stop sending the tracking signals (e.g., after a selected time of no motion being detected, the transmitter can be deactivated). The tracking signals captured by the dispensers can be sent, together with location information for the capturing dispensers and the times and dates of such captures to enable mapping and tracking of the movements of the piece of equipment and a last known location thereof when the piece of equipment is stopped.

In embodiments, the badges carried by persons moving about the facility can be configured to send badge signals on a substantially constant basis. Alternatively, the badge signals can be sent in response to a transmission or signal from a dispenser, such as when the person enters a room, their badge can detect signals from one or more dispensers searching or querying for a badge, and in response transmit the badge signals.

In addition, in embodiments, the badge signals can comprise sets of signals sent in series or as separate pulses, which signals can include a signature or other identifying information identifying the badge (and by association, the person carrying that badge), and one or more additional signals that can be of different strengths, signal lengths that limit their transmission distance, different frequencies, or with other characteristics. The additional signals can be used to determine with enhanced accuracy the movements of the person (e.g., if a doctor got close enough to a patient’s bed to require use of a dispenser).

In some further embodiments, the dispensers can include one or more receivers configured to receive the tracking and/or badge signals when the equipment and/or personnel come within a selected area, distance, zone or range of the dispenser sufficient to be detected by the one or more receivers. The dispensers also can be configured to monitor use thereof by an individual detected within the selected area, distance, zone or range as part of a compliance system to ensure sufficient use of the dispenser(s) as required by a facility protocol or procedure.

In embodiments, the dispensers can collect and send one or more information packets including information identifying the dispenser and/or its location in the facility, and a record of detected/captured tracking signals and other signals identifying specific pieces of equipment and/or personnel, as well as, in some embodiments, usage information confirming use of the dispenser by an identified individual. Such information packets can be transmitted periodically, such as at selected time intervals or after detection of a piece of equipment or individual and/or use of the dispenser.

According to other aspects of the disclosure, a system is provided, comprising: a plurality of tracking systems, each tracking system linked to an associated piece of equipment and configured to transmit one or more tracking signals including signature information identifying the associated piece of equipment; wherein each tracking system is configured to detect movement of the associated piece of equipment, and in response to detection of the movement, emit the one or more tracking signals as the associated piece of equipment moves about a facility; a plurality of dispensers positioned at selected locations within the facility, each dispenser comprising: a dispensing mechanism configured to dispense a fluid or a sheet material; at least one receiver configured to receive the one or more tracking signals emitted by the tracking systems when the piece of equipment; a control system comprising a controller in communication with the dispensing mechanism and the at least one receiver; wherein the controller includes programming configured to: control the dispensing mechanism to dispense an amount of the fluid or the sheet material upon detection of a user initiating a dispensing operation; and transmit one or more information packets to a facility server, facility control, cloud server, network, mobile application, mobile or wireless or combinations thereof.

In embodiments, each information packet can include a location identifier for the dispenser and signature information for at least one piece of equipment detected within prescribed range, area or zone of the dispenser to facilitate identifying, logging, mapping, tracking, or combinations thereof, of the movement and/or location of the pieces of equipment throughout the facility.

In embodiments, each tracking system comprises at least one sensor configured to detect the movement of its associated piece of equipment and at least one transmitter configured to transmit the signature information.

In some embodiments, each tracking system further includes at least one processor configured to receive a movement signal from the at least one sensor indicating movement of the associated piece of equipment and activate the at least one transmitter; wherein when the associated piece of equipment is determined to have stopped or be in a non-moving state, the at least one transmitter is placed in an inactive state.

In embodiments, the facility comprises a medical facility, and wherein the pieces of equipment comprise one or more of patient mobility aids, beds, monitors, portable diagnostic equipment, , portable treatment and life support equipment, and portable surgical equipment.

In embodiments, each dispenser further can comprise at least one activation sensor in communication with the controller and configured to detect an activation gesture by the user and send an activation signal or prompt to the controller to initiate the dispensing operation, and a pumping or feeding system for dispensing the fluid or the sheet material during the dispensing operation.

In embodiments, one or more of the dispensers further comprises a power management system including at least one passive infrared radiation (PIR) sensor, at least one radar sensor, or a combination thereof, in communication with the control system or the at least one activation sensor.

In some embodiments, the at least one PIR sensor is configured to connect the controller, the at least one receiver, the at least one transmitter, the at least one activation sensor, and the pumping or feeding system to a power source when the at least one PIR sensor captures infrared radiation of one or more individuals within the prescribed detection range, area, or zone of the PIR sensor; and, in embodiments, the controller, the at least one receiver, the at least one transmitter, the at least one activation sensor, the pumping or feeding system generally will be placed in a substantially powered off state until the at least one PIR sensor captures the infrared radiation.

In embodiments, the system can further comprise a compliance system comprising a plurality of badges carried by a plurality of individuals, each badge being configured to transmit a series of signals including information identifying each badge; wherein the signals are transmitted for a distance, with a selected signal strength, or combinations thereof, to be received by a dispenser when the badge moves within a selected zone, area, proximity or range of the dispenser.

In embodiments, each information packet transmitted by the at least one transmitter of each dispenser includes the location identifier for the dispenser and can include the signature information for at least one piece of equipment, the information identifying an individual associated with each badge, or combinations thereof, detected within the selected zone, area, proximity, or range of the dispenser during a prescribed period.

According to another aspect of the disclosure, a method is provided, comprising: tracking and/or locating pieces of equipment within a facility, comprising: detecting movement of a piece of equipment by a tracking system linked to the piece of equipment; upon detection of the movement of the piece of equipment, emitting a plurality of tracking signals using the tracking system, the tracking signals including signature information identifying a piece of equipment linked to the tracking system; and as the piece of equipment moves about a location, detecting at least one tracking signal emitted by the tracking system at each of a plurality of dispensers as the piece of equipment moves within a first zone, area, proximity, or range thereof; and tracking compliance with protocols by individuals moving about the facility, comprising: detecting one or more signals emitted by badges carried by the individuals at one or more dispensers of the plurality dispensers when the badges move within a second zone, area, proximity, or range of the one or more dispensers of the plurality of dispensers; wherein the one or more signals emitted by each badge includes identifying information for identifying the individual carrying the badge.

In embodiments, upon detection of at least one badge signal emitted by at least one badge or one or more tracking signals at a dispenser, determining usage of the dispenser by an individual carrying the at least one badge; and transmitting one or more information packets from each dispenser of the plurality of dispensers to a facility server, facility control system, cloud server, network, mobile application, or combinations thereof.

In embodiments, each information packet includes a location identifier for the dispenser and can include one or more of signature information for at least one piece of equipment received by the dispenser and usage information for the dispenser.

In embodiments, the usage information can include information identifying each badge detected by the dispenser, information indicating activation and usage of the dispenser by the individual carrying each badge detected by the dispenser, dispenser status information or combinations thereof.

In embodiments, determining usage of the dispenser comprises activating the dispenser to initiate a dispensing operation for dispensing a fluid or sheet material in response to an activation prompt from the individual; receiving one or more additional signals from the badge carried by the individual by the dispenser; and determining if the individual used the dispenser sufficient to comply with an established protocol for the facility.

In embodiments, each dispenser includes a controller, a feed mechanism, at least one activation sensor, one or more receivers configured to receive the signals transmitted by the badges, and a power management system including at least one PIR sensor. In some embodiments, the method can further comprise: detecting infrared radiation of one or more individuals within a prescribed detection range, area, or zone of the dispenser with the at least one PIR sensor of the power management system of the dispenser; connecting the at least one activation sensor to power to place the at least one activation sensor in a powered-on state; if the at least one activation sensor detects an individual moving within a selected activation zone of at least one dispenser of a dispensing system: connecting a controller of the dispenser to power to place the controller in a powered-on state; transmitting an activation signal to the controller; connecting a dispensing mechanism of the dispenser to power to place the dispensing mechanism in a powered-on state; and initiating a dispensing operation to dispense an amount of fluid or sheet material.

In addition, in embodiments, when movement of the piece of equipment is not detected by the tracking system linked thereto, the tracking system will cease sending tracking signals; and further comprising tracking movement of the piece of equipment within the facility based on locations of each dispenser at which the piece of equipment was detected; and determining a location of the piece of equipment based on a last dispenser location at which the piece of equipment was detected.

According to another aspect, a facility monitoring system is provided, comprising: a plurality of dispensers positioned at known locations throughout the facility, each of the dispensers comprising: a dispensing mechanism operable for dispensing a fluid or sheet material; a control system in communication with the dispensing mechanism, and including: programming to control the dispensing mechanism to perform a dispensing operation for dispensing an amount of the fluid or sheet material; wherein the control system comprises a controller, at least one activation sensor configured to transmit an activation prompt to the controller to initiate the dispensing operation, and at least one transmitter; and a plurality of tracking systems each linked to an associated piece or equipment and each including a controller configured to detect motion by the piece of equipment and a transmitter configured to transmit one or more tracking signals in response. In addition, in embodiments, a series of badges can be carried by individuals moving about the facility, each of the badges including one or more transmitters configured transmit a series of signals including information identifying each badge.

In embodiments of the facility monitoring system, as the pieces of equipment and the individuals carrying the badges move about the facility, the tracking signals and the signals from the badges are received by one or more of the dispensers; and wherein the controller of each dispenser includes programming configured to transmit one or more information packets to a facility server, facility control server, cloud server, network, mobile application, mobile or other wireless device, or combinations thereof.

In embodiments of the facility monitoring system, the information packets can include information identifying a location of the dispenser, and one or more tracking signals detected by the dispenser, information identifying one or more badges detected by the dispenser, dispenser status information, or combinations thereof.

In embodiments of the facility monitoring system, at least some of the dispensers further can comprise a power management system including at least one detection sensor configured to transition one or more of the at least one activation sensor, the controller, the transmitter, and the dispensing mechanism to a powered-on state when the at least one detection sensor detects a presence of one or more individuals within a prescribed detection range, area, or zone.

In embodiments of the facility monitoring system, the power management system is configured to initially connect the activation sensor of the dispenser to power prior to connecting the controller, dispensing mechanism, and the transmitter to power.

In some embodiments, the at least one detection sensor can comprise a PIR sensor configured to capture infrared radiation of the one or more individuals within the prescribed detection range, area, or zone.

In other embodiments, the at least one detection sensor can comprise a radar sensor configured to capture movement of the one or more individuals within the prescribed detection range, area, or zone.

In embodiments of the facility monitoring system, at least the controller and the dispensing mechanism can remain disconnected from power until the at least one activation sensor has transmitted the activation prompt to the controller to initiate a dispensing operation.

In embodiments of the facility monitoring system, the power management system can be further configured to transition the transmitter to a powered-on state upon capturing infrared radiation of one or more individuals within the prescribed detection range, area, or zone.

In embodiments of the facility monitoring system, the transmitter is in communication with the power management system and includes a controller; and wherein the at least one detection sensor is configured to connect the at least one transmitter to the power source when the at least one detection sensor detects one or more individuals within the prescribed detection range, area, or zone, and wherein upon connection to power, the at least one transmitter can transmit the information packet.

In embodiments of the facility monitoring system, the power management system can be configured to periodically transition the transmitter to a powered-on state whereupon the transmitter can transmit an information packet without detection of infrared radiation of one or more individuals within a prescribed detection range, area, or zone by a PIR sensor or movement of the one or more individuals within the prescribed detection range, area, or zone by a radar sensor.

In embodiments, each of the dispensers further can comprise at least one receiver configured to receive the tracking signals transmitted by the tracking systems, and at least one receiver configured to receive the signals transmitted by the badges.

In embodiments of the facility monitoring system, the dispenser status information comprises dispenser usage and diagnostic information. In some embodiments, the dispenser usage and diagnostic information can comprise one or more detected fault conditions, , fluid or sheet material supply information, power level information, power usage information, or combinations thereof.

In some embodiments of the facility monitoring system, the control system of each dispenser is configured to monitor and receive a series of signals from at least one badge within a threshold range, zone, area or proximity to the dispenser and determine whether a dispensing operation was initiated while the series of signals were detected, and if a dispensing operation is initiated, monitor and receive signals from the at least one badge at multiple times during a dispensing operation to determine usage of the dispenser by an individual carrying the at least one badge.

In embodiments, the facility comprises a medical facility, and wherein the pieces of equipment comprise one or more of patient mobility aids, beds, monitors, portable diagnostic equipment, portable diagnostic equipment, portable treatment and life support equipment, and portable surgical equipment.

In embodiments, each badge is configured to transmit a plurality of signals, each of the signals being transmitted at a predetermined distance or at a predetermined signal strength, and wherein the control system of each dispenser is configured to determine a plurality of positions or movements of each individual within the selected target range based on the distance or signal strength of the signals received from a badge carried by the individual.

According to some aspects, a monitoring system is provided, comprising a plurality of tracking systems configured to transmit one or more tracking signals including signature information identifying an associated piece of equipment; a plurality of dispensers positioned at selected locations within the facility, each dispenser configured to dispense a fluid or a sheet material and having at least one receiver configured to receive the one or more tracking signals transmitted by the tracking systems and a control system configured to control operation of the dispenser to dispense an amount of the fluid or the sheet material upon detection of a user initiating a dispensing operation; and transmit an information packet including a location identifier for the dispenser and information at least one piece of equipment detected moving within a range, area or zone of the dispenser.

In embodiments, the information packet is transmitted to a facility server, facility control, cloud server, network, mobile application, mobile or other wireless device, or combinations thereof.

In some embodiments, the facility comprises a medical facility, and wherein the pieces of equipment comprise one or more of patient mobility aids, beds, monitors, portable diagnostic equipment, , portable treatment and life support equipment, and portable surgical equipment.

In embodiments, each tracking system comprises at least one sensor configured to detect the movement of its associated piece of equipment and activate at least one transmitter to transmit the one or more tracking signals as the associated piece of equipment moves about a facility.

In embodiments, wherein each tracking system further includes at least one processor configured to receive a movement signal from the at least one sensor indicating movement of the associated piece of equipment and activate the at least one transmitter; wherein when the associated piece of equipment is determined to have stopped or be in a non-moving state, the at least one transmitter is placed in an inactive state.

In some embodiments, the system further comprises a compliance system including a plurality of badges carried by a plurality of individuals, each badge being configured to transmit a series of signals including information identifying each badge; wherein the signals are transmitted for a distance, with a selected signal strength, or combinations thereof, to be received by a dispenser when the badge moves within a selected zone, area, proximity or range of the dispenser.

According to some other aspects, a system is provided, comprising a plurality of tracking systems, each tracking system linked to an associated piece of equipment and configured to transmit one or more tracking signals including signature information identifying the associated piece of equipment; a plurality of dispensers positioned at selected locations within the facility, each dispenser comprising: a dispensing mechanism configured to dispense a fluid or a sheet material; at least one receiver configured to receive the one or more tracking signals emitted by the tracking systems; and a control system in communication with the dispensing mechanism and the at least one receiver and including programming configured to: control the dispensing mechanism to dispense an amount of the fluid or the sheet material upon detection of a user initiating a dispensing operation; and transmit an information packet including a location identifier for the dispenser and signature information for at least one piece of equipment detected moving within a prescribed range, area or zone of the dispenser to a facility server, facility control, cloud server, network, mobile application, mobile or other wireless device, or combinations thereof.

In embodiments of the system, each tracking system can comprise at least one sensor configured to detect the movement of its associated piece of equipment and activate at least one transmitter to transmit the one or more tracking signals as the associated piece of equipment moves about a facility. In addition, in embodiments, when the associated piece of equipment is determined to have stopped or be in a non-moving state, the at least one transmitter is placed in an inactive state.

In embodiments of the system, one or more of the dispensers further comprises a power management system including at least one passive infrared radiation (PIR) sensor configured to capture infrared radiation or at least one radar sensor configured to capture movement, the at least one PIR sensor or at least one radar sensor being in communication with the control system of at least one dispenser; and wherein the at least one PIR sensor or at least one radar sensor is configured to connect the control system, the at least one receiver, the at least one transmitter, the at least one activation sensor, the dispensing mechanism, or combinations thereof, to a power source upon detection of one or more individuals within a prescribed detection range, area, or zone of the PIR sensor or radar sensor.

In embodiments, the system further can comprise a compliance system comprising a plurality of badges carried by a plurality of individuals, each badge being configured to transmit a series of signals including information identifying each badge; wherein the signals are transmitted for a distance, with a selected signal strength, or combinations thereof, to be received by a dispenser when the badge moves within a selected zone, area, proximity or range of the dispenser.

In embodiments of the system, an information packet transmitted by the at least one transmitter of each dispenser can include information identifying each badge detected within the selected zone, area, proximity, or range of the dispenser during a prescribed period.

According to another aspect, a method of tracking and/or locating pieces of equipment within a facility can comprise: detecting movement of a piece of equipment by a tracking system linked to the piece of equipment; upon detection of the movement of the piece of equipment, emitting a plurality of tracking signals, the tracking signals including signature information identifying a piece of equipment linked to the tracking system; and as the piece of equipment moves about the facility, detecting at least one tracking signal emitted by the tracking system at each of a plurality of dispensers; and at least periodically transmitting one or more information packets from each dispenser of the plurality of dispensers to a facility server, facility control, cloud server, network, mobile application, mobile or other wireless device, or combinations thereof; and wherein each information packet includes a location identifier for the dispenser, and one or more of signature information for at least one piece of equipment received by the dispenser and usage information for the dispenser.

In embodiments, the method can further comprise detecting one or more signals emitted by badges carried by individuals moving about the facility at one or more dispensers of the plurality dispensers; wherein the one or more signals emitted by each badge include information identifying the badge; upon detection of at least one signal emitted by at least one badge at a dispenser, determining usage of the dispenser by an individual carrying the at least one badge.

In embodiments of the method, determining usage of the dispenser comprises activating the dispenser to initiate a dispensing operation for dispensing a fluid or sheet material in response to an activation prompt; receiving one or more additional signals from the at least one badge by the dispenser; and determining if the individual carrying the at least one badge used the dispenser sufficient to comply with a protocol for the facility.

In some embodiments, each dispenser includes a controller, a dispensing mechanism, at least one activation sensor, one or more receivers configured to receive the signals transmitted by the badges, and a power management system including at least one PIR sensor; and the method can further comprise detecting infrared radiation of one or more individuals within a prescribed detection range, area, or zone of the dispenser with the at least one PIR sensor; placing the at least one activation sensor in a powered-on state; if the at least one activation sensor detects an individual moving within a selected activation zone of at least one dispenser of a dispensing system: placing the controller of the dispenser in a powered-on state, transmitting an activation signal or prompt to the controller; and initiating a dispensing operation to dispense an amount of fluid or sheet material.

In embodiments, the method can further comprise tracking movement of the piece of equipment within the facility based on a location of each dispenser at which the piece of equipment was detected; and determining a location of the piece of equipment based on a last dispenser location at which the piece of equipment was detected.

In some embodiments of the method, when movement of the piece of equipment is not detected by the tracking system linked thereto, discontinuing sending tracking signals.

Another aspect can include a facility monitoring system comprising: a plurality of dispensers positioned throughout the facility, each of the dispensers comprising: a dispensing mechanism operable for dispensing a fluid or sheet material; a control system including a controller in communication with the dispensing mechanism, at least one activation sensor, and at least one transmitter; a plurality of tracking systems each linked to an associated piece or equipment and configured to transmit one or more tracking signals; a series of badges configured to be carried by individuals moving about the facility, each of the badges including one or more transmitters configured transmit a series of signals including information identifying each badge; wherein as the pieces of equipment and the individuals carrying the badges move about the facility, the tracking signals and the signals from the badges are received by one or more of the dispensers, wherein the controller of each dispenser includes programming configured to transmit one or more information packets to a facility server, facility control, cloud server, network, mobile application, mobile or other wireless device, or combinations thereof; and wherein the information packets include information identifying a location of the dispenser, and one or more tracking signals detected by the dispenser, information identifying one or more badges detected by the dispenser, dispenser status information, or combinations thereof.

In embodiments of the facility monitoring system, at least some of the dispensers further comprise a power management system including at least one PIR sensor or at least one radar sensor configured to connect one or more of the at least one activation sensor, the controller, the transmitter, and the dispensing mechanism to power when the presence of one or more individuals is detected within a prescribed detection range, area, or zone by the at least one PIR sensor or at least one radar sensor.

In embodiments of the facility monitoring system, the at least one transmitter is in communication with the power management system; and wherein the at least one PIR sensor or at least one radar sensor is configured to connect the at least one transmitter to the power source when the at least one PIR sensor or at least one radar sensor detects the presence of one or more individuals within the prescribed detection range, area, or zone, and wherein upon connection to the power supply, the at least one transmitter is configured to at least periodically transmit the dispenser status information.

In embodiments of the facility monitoring system, each of the dispensers can further comprise at least one receiver configured to receive the tracking signals transmitted by the tracking systems, and at least one receiver configured to receive the signals transmitted by the badges.

In some embodiments of the facility monitoring system, each tracking system can comprise a controller configured to detect motion of the piece of equipment and activate a transmitter to transmit the one or more tracking signals in response to detection of the movement of the piece of equipment is detected.

In embodiments of the facility monitoring system, each tracking system can comprise a vibration detector configured to detect movement of the piece of equipment and a transmitter configured to transmit the one or more tracking signals in response to detection of the movement of the piece of equipment is detected.

In embodiments of the facility monitoring system, the control system of each dispenser is configured to monitor and receive a series of signals from at least one badge within a threshold range, zone, area or proximity to the dispenser and determine whether a dispensing operation was initiated while the series of signals were detected, and if a dispensing operation is initiated, monitor and receive signals from the at least one badge at multiple times during a dispensing operation to determine usage of the dispenser by an individual carrying the at least one badge.

In embodiments of the facility monitoring system, the facility can comprise a medical facility, and wherein the pieces of equipment comprise one or more of patient mobility aids, beds, monitors, portable diagnostic equipment, portable diagnostic equipment, portable treatment and life support equipment, and portable surgical equipment.

In embodiments, the dispensing system(s) of the facility monitoring system can include a series of lead and drone dispensers configured for bidirectional or cross-communication between at least the drone dispensers and the lead dispenser; and in some embodiments, also between various ones of the drone dispensers. In embodiments, each of the dispensers of the dispensing system can be configured to send dispenser usage and diagnostic information at least to the lead dispenser, and in some can share such information with one or more drone dispensers as well. In embodiments, the communications can be periodic, or can be in response to a request from the lead dispenser, which could also include instructions from the lead dispenser, such as for a reset or for the collection of requested dispenser usage and diagnostic information, etc.

Still further, the bidirectional cross-communication between dispensers enables enhanced monitoring and generation of dispenser diagnostic information needed from a maintenance or servicing perspective, which can be developed and accessed by authorized personnel generally on request. In addition, in embodiments, such cross-communication between dispensers can enable enhanced power management of the dispenser operations, for example, enabling selective shut down and powering of various dispensers. As a result, the dispensing systems can maximize the monitoring, collection of information, and the total amount and types of information collected and made accessible to management/supervisory personnel to provide increased efficiencies in operation, substantial reductions in energy costs and service costs for the dispensers, and increased efficiency in servicing and understanding usage of the dispensers, including planning and ordering of supplies and deployment of personnel for servicing the dispensers, as well as helping to facilitate the integration and efficiencies in operation of equipment and compliance monitoring and tracking within a facility.

Still other aspects, embodiments, and advantages of the foregoing exemplary aspects and embodiments, are discussed in detail below. Moreover, it is to be understood that both the foregoing information and the following detailed description are merely illustrative examples of various aspects and embodiments and are intended to provide an overview or framework for understanding the nature and character of the claimed aspects and embodiments. Accordingly, these and other objects, along with advantages and features of the present disclosure herein disclosed, will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain the principles of the embodiments discussed herein. No attempt is made to show structural details of this disclosure in more detail than can be necessary for a fundamental understanding of the exemplary embodiments discussed herein and the various ways in which they can be practiced. It further will be understood that the various features of the drawings discussed below are not necessarily drawn to scale. Dimensions of various features and elements in the drawings can be expanded or reduced to more clearly illustrate various aspects and embodiments of the dispensing systems and methods according to the principles of the present disclosure.

FIGS. 1A-1B are perspective views of an example embodiment of a dispenser of a dispensing system according to principles of the present disclosure, with the dispenser shown as being operable for dispensing a sheet material.

FIG. 1C is a perspective view of the dispenser of FIGS. 1A-1B, taken in partial cross-section.

FIG. 2A is a perspective illustration of an example of an additional embodiment of a dispenser of a dispensing system according to the principles of the present disclosure, with the dispenser being configured for selectively dispensing sheet materials from one or two supplies of sheet material.

FIG. 2B is a perspective view of the dispenser of FIG. 2A, with a front portion of the housing being removed.

FIG. 2C is a perspective view illustrating an embodiment of the loading of supplies of sheet material in the dispenser of FIG. 2A.

FIG. 2D is a perspective view of an embodiment of a dispensing mechanism for use with the dispenser of FIG. 2A.

FIG. 2E is a perspective view illustrating an embodiment of a feeding operation for feeding amounts of sheet material from alternate ones of the supplies of sheet material with the dispenser of FIG. 2A.

FIG. 2F is a side elevational view taken in cross-section, of an embodiment of a dispensing mechanism.

FIG. 2G is a perspective view taken in cross-section, of the dispensing mechanism of FIG. 2F.

FIGS. 3A-3B are side elevational view illustrating another example embodiment of a dispenser of a dispensing system according to principles of the present disclosure, with the dispenser shown as being operable for dispensing fluids.

FIG. 3C is an exploded perspective view of the dispenser of FIGS. 3A-3B.

FIG. 4A illustrates a further example embodiment of a dispenser of a dispensing system according to principles of the present disclosure, with the dispenser shown as being operable for dispensing a sheet material from a plurality of supplies of sheet material.

FIG. 4B shows an example embodiment of an activation sensor.

FIGS. 4C-4D are side cross-sectional views illustrating embodiments of a target activation zone or area for an activation sensor of a dispenser according to the principles of the present disclosure.

FIGS. 5A-5B are perspective views illustrating a cassette or drive mechanism for a dispenser and further illustrating an additional embodiment of a target activation zone or area for an activation sensor of a dispenser according to the principles of the present disclosure.

FIG. 6A is a schematic perspective view of an additional example embodiment of a dispenser of a dispensing system according to principles of the present disclosure, with the dispenser shown as being operable for dispensing a sheet material from a plurality of supplies of sheet material.

FIG. 6B illustrates a portion of the dispenser of FIG. 6A, showing a PIR sensor of a power management system and an IR beam from an activation sensor according to the principles of the present disclosure.

FIGS. 6C-6D are side cross-sectional views illustrating embodiments of a target activation zone or area for an activation sensor of a dispenser according to the principles of the present disclosure.

FIG. 6E is a perspective view illustrating a further embodiment of a target activation zone or area for an activation sensor of a dispenser according to the principles of the present disclosure.

FIG. 6F is a side view of the embodiment of a target activation zone or area for an activation sensor of a dispenser shown in FIG. 6C.

FIGS. 7A-7D are schematic views illustrating various embodiments of operative components of a dispensing system with a power management system according to the principles of the present disclosure.

FIGS. 8A-8E are schematic views illustrating an example embodiment of a dispensing system with a power management system according to the principles of the present disclosure, illustrating the dispensing system in different operating states including power consumption rates of the dispensing system in such operating states.

FIGS. 9A-9E are schematic views illustrating another example embodiment of a dispensing system with a power management system according to the principles of the present disclosure, illustrating the dispensing system as being configured for dispensing materials from multiple supplies in different power consumption states.

FIG. 10 is a schematic illustration of an example embodiment of a power management system including an example embodiment of a detection sensor thereof, including a passive infrared radiation (PIR) sensor, that can be used in the dispensing system with a power management system according to the principles of the present disclosure.

FIG. 11 is a schematic illustration of an example embodiment of a compliance and tracking system and an example method of operation thereof in accordance with the principles of the present disclosure.

FIG. 12 is a schematic illustration of an example embodiment of a tracking system in accordance with the principles of the present disclosure.

FIGS. 13A-13B are schematic illustrations of example embodiments of compliance systems, illustrating the transmission of identifying signals from one or more badges that are received by a dispenser, in accordance with the principles of the present disclosure.

FIG. 14 is a schematic illustration showing an example compliance and tracking system in a facility, in accordance with the principles of the present disclosure

### DETAILED DESCRIPTION

The present disclosure relates to dispensing systems and methods of operation/use thereof, and in embodiments, to dispensing systems including dispensers configured to selectively dispense sheet materials or fluid or liquid materials. In embodiments, the fluid or liquid materials can comprise soaps, sanitizers, and/or other fluids or liquids that can be provided and stored or contained within a supply, such as a container that can loaded into the dispenser and from which the fluid can be and selectively drawn and dispensed. In other embodiments, sheet materials such as, without limitation, paper towels, tissues and other, similar materials, can be loaded into the dispenser in rolls, with the sheet materials being progressively drawn from the rolls during a dispensing operation.

The dispensing systems, dispensers and various features, assemblies and mechanisms thereof, as well as various methods of operating such dispensing systems, dispensers, assemblies and mechanisms for dispensing fluid or sheet materials according to the present disclosure can be understood more readily by reference to the following detailed description, examples, drawings, and claims, and their previous and following description. However, before the present devices, systems, and/or methods are disclosed and described, it is to be understood that this disclosure is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the dispensing systems, dispensers and methods is provided as an enabling teaching of such dispensing systems, dispensers and methods according to the principles of the present disclosure. To this end, those skilled in the relevant art will recognize and appreciate that many changes can be made to the various aspects of the disclosure described herein, while still obtaining the beneficial results of the dispensing systems, dispensers and methods of the present disclosure. It will also be apparent that some of the desired benefits of the dispensing systems, dispensers and methods of the present disclosure can be obtained by selecting some of the features of the systems, devices and methods of present without utilizing other features. Accordingly, those who work in the art will recognize that many modifications and adaptations to the dispensing systems, dispensers and methods described herein are possible and can even be desirable in certain circumstances and are a part of the dispensing systems, dispensers and methods of the present disclosure. Thus, the following description is provided as illustrative of a system, and various operative components, systems and equipment thereof, including equipment tracking systems, compliance systems, dispensing systems, dispensers and methods of operation thereof according to the principles of the present disclosure and not in limitation thereof.

As used throughout, the singular forms “a,” “an” and “the” include plural referents unless the context clearly dictates otherwise. Thus, for example, reference to “a chamber” can include two or more such chambers unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “can,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular embodiment.

The phraseology and terminology used herein is for the purpose of description and should not be regarded as limiting. As used herein, the term "plurality" refers to two or more items or components. The terms "comprising," "including," "carrying," "having," "containing," and "involving," whether in the written description or the claims and the like, are open-ended terms, i.e., to mean "including but not limited to." Thus, the use of such terms is meant to encompass the items listed thereafter, and equivalents thereof, as well as additional items. Only the transitional phrases "consisting of" and "consisting essentially of," are closed or semi-closed transitional phrases, respectively, with respect to any claims. Use of ordinal terms such as "first," "second," "third," and the like in the claims to modify a claim element does not by itself connote any priority, precedence, or order of one claim element over another or the temporal order in which acts of a method are performed but are used merely as labels to distinguish one claim element having a certain name from another element having a same name (but for use of the ordinal term) to distinguish claim elements.

Disclosed are components that can be used to perform the disclosed methods and systems. These and other components are disclosed herein, and it is understood that when combinations, subsets, interactions, groups, etc. of these components are disclosed that while specific reference to each various individual and collective combinations and permutation of these cannot be explicitly disclosed, each is specifically contemplated and described herein, for all methods and systems. This applies to all aspects of this application including, but not limited to, steps in the disclosed methods. Thus, if there are additional steps that can be performed, it is understood that each of these additional steps can be performed with any specific embodiment or combination of embodiments of the disclosed methods.

The present methods and systems can be understood more readily by reference to the following detailed description of preferred embodiments and the examples included therein and to the Figures and their previous and following description.

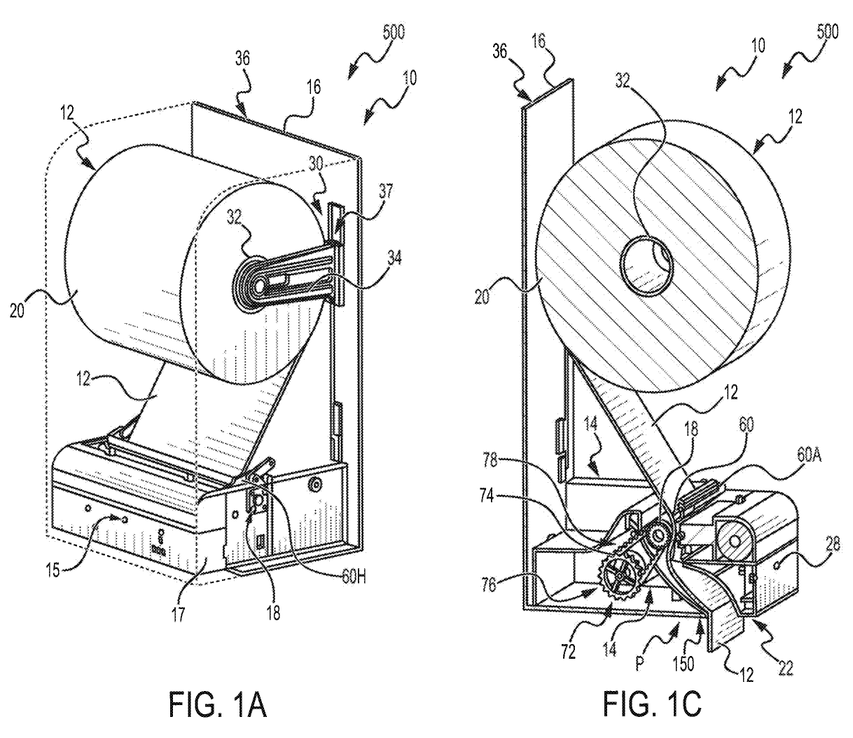

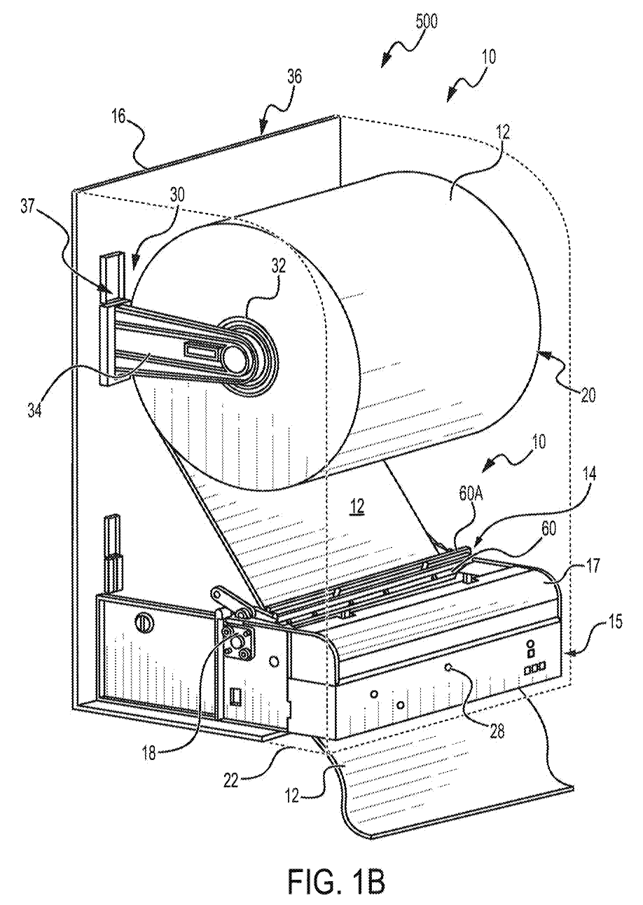

As generally illustrated in FIGS. 1A-9E embodiments of the present disclosure are directed to systems and methods for cooperatively monitoring operations in a facility, such as, for example, a medical facility, and which, in embodiments, can comprise an integrated system in which one or more dispensing systems 500, which will include one or more dispensers 10, and, and generally a plurality of dispensers 10 in communication with a compliance system 1000 for monitoring compliance with dispenser use protocols or policies, and a system 1050 for monitoring movement of equipment in a facility, including a series tracking systems linked to associated pieces of equipment.

In embodiments, the dispensers further can be configured with an operating architecture that can include a plurality of controllers (e.g., processors or CPUs) for controlling operation of the various operative components of the dispensers. For example, as discussed further below, in embodiments, the dispensers can include a dispenser controller, one or more activation sensors, a dispensing mechanism, one or more transmitters, one or more receivers, supply monitoring devices, and one or more power management systems, each of which can include a dedicated controller/CPU/processor (though some components such as the dispensing mechanism can be linked to and can be operated by , for example, the dispenser controller). The different controllers can be in bidirectional communication with each other, can be operable independently of the remaining components to enable enhanced load shedding, helping to minimize power drain.

Still further, in embodiments, the dispensers can be in bidirectional communication with one or more additional dispensers within the facility, such that some dispensers can receive instructions to place them in a powered-on state. For example, a signal can be sent from a lead dispenser to a power management system of one or more dispensers linked/in communication therewith to cause the power management system of each of such one or more dispensers to be placed in a powered-on or awake state, while the remaining components of the dispenser can remain in a powered-down or off state until the power management system detects the presence of an individual or a tracking system of a piece of equipment within a prescribed detection range, zone, area, or distance.

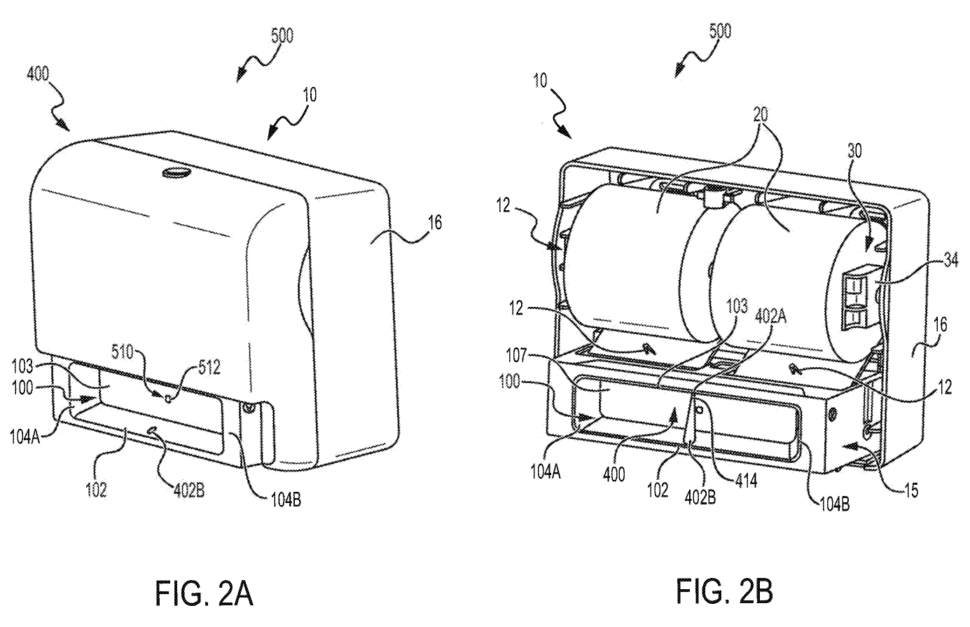

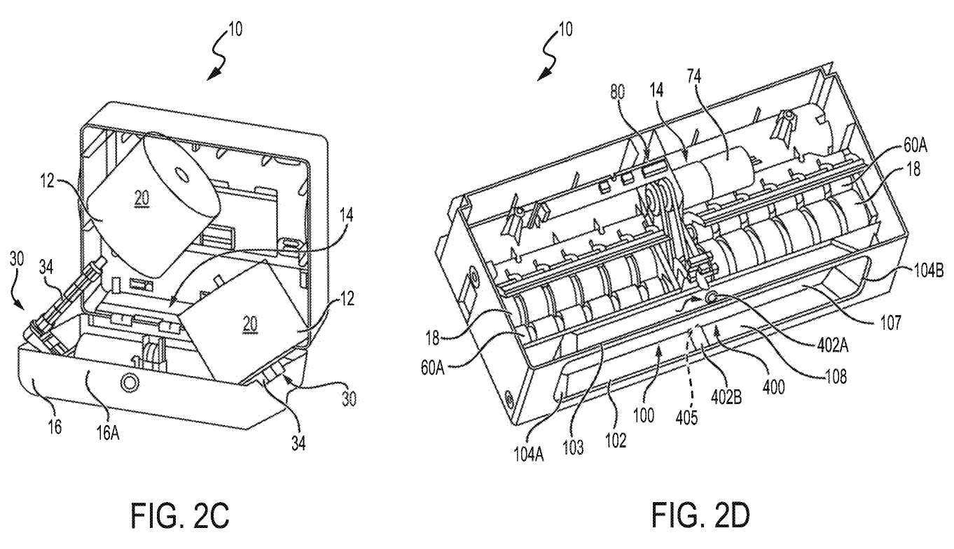

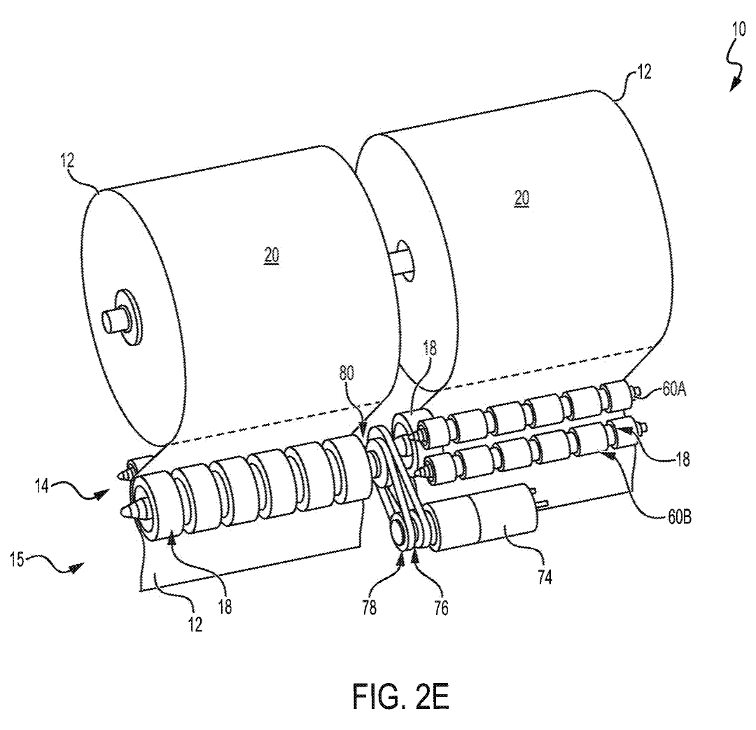

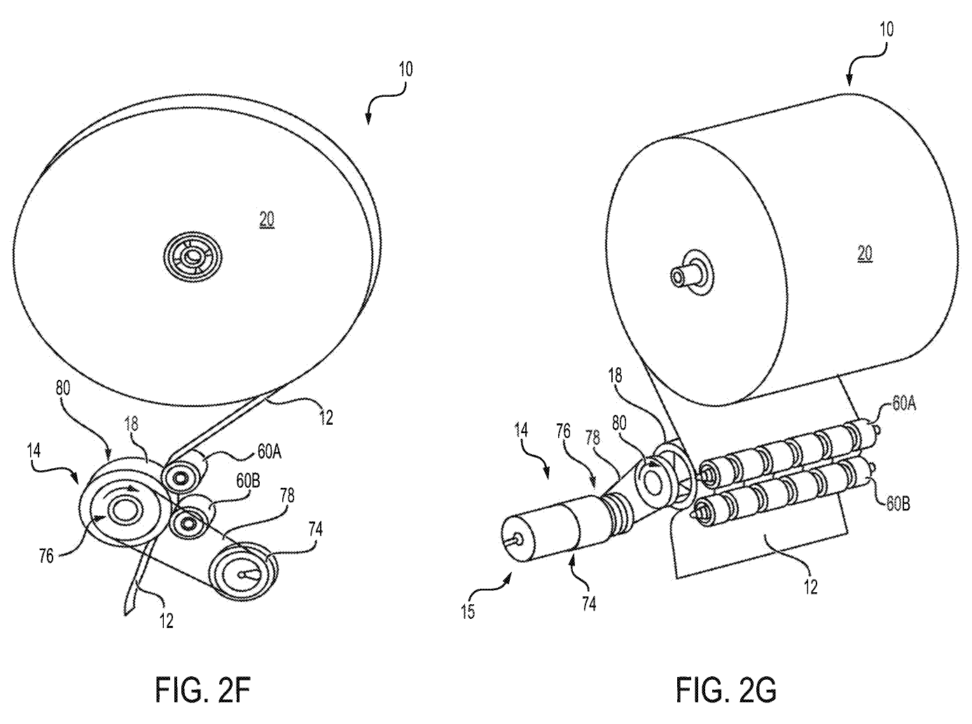

As illustrated in FIGS. 1A-1B, in embodiments, the dispenser generally can comprise an automated dispenser 10 for feeding or dispensing a selected material. For example, in some embodiments, the dispenser can be configured to dispense a sheet material 12, which, in embodiments, can include a dispenser 10 adapted to dispense a sheet material from a single supply as illustrated in FIGS. 1A-1C, or from multiple (e.g., two) supplies of sheet materials such as illustrated in FIGS. 2B-2G. In other embodiments, the dispenser can comprise a fluid dispenser 300 configured for dispensing fluid or liquid materials.

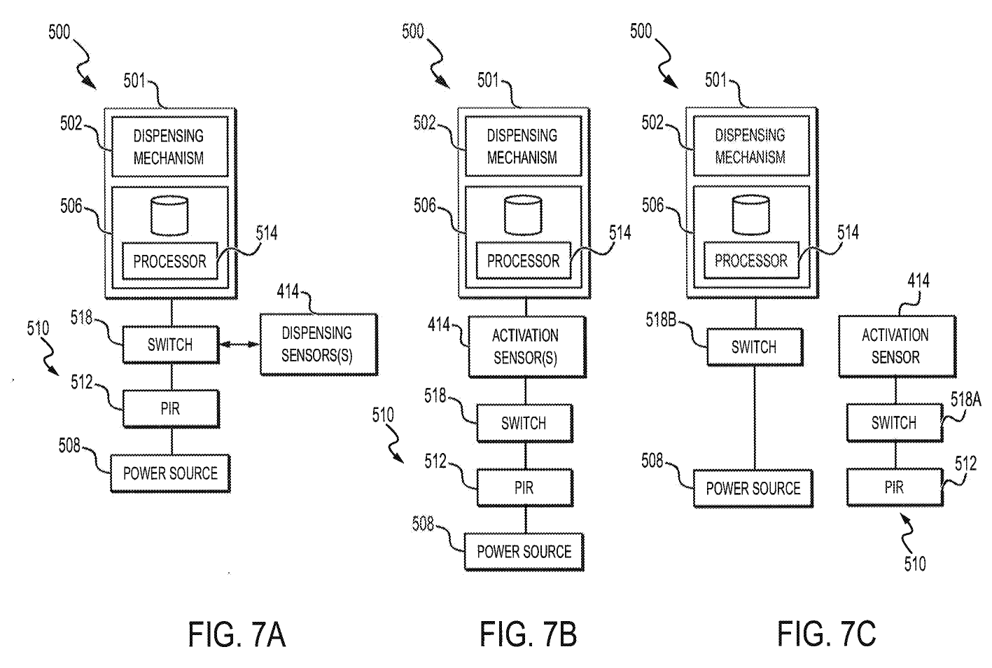

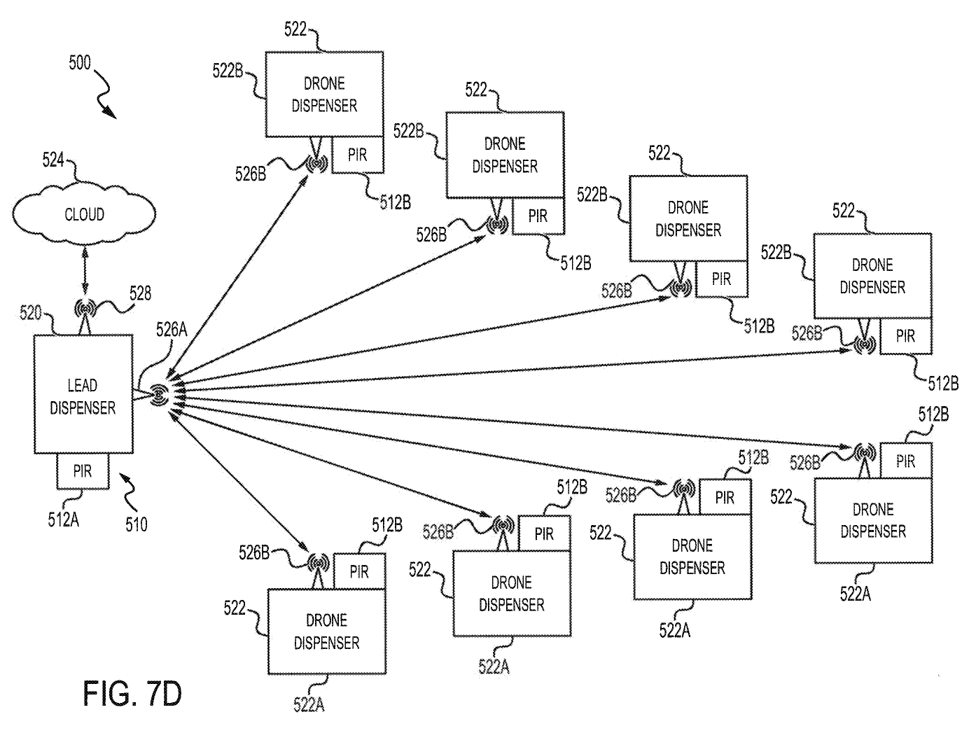

In addition, in embodiments, such as shown in FIGS. 7A-7D, the dispensing system 500 can include one or more dispensers. For example, in some embodiments, such as illustrated in FIG. 7D, the dispensing system 500 can include a plurality of dispensers that can be located at different selected locations within a facility. In embodiments, the dispensers of the dispensing system can be located, such as, for example, within bathrooms, patient rooms, personnel stations, etc. of a facility such as a hospital, clinic or other, similar facility. In other embodiments, the dispensers of the dispensing system can be arranged in sets or series that can include a lead dispenser and one or more drone dispensers that communicate with the lead dispenser as illustrated in FIG. 7D.

In embodiments, the dispenser may be recessed into a wall with a lower profile or configuration to help reduce a footprint of the dispenser or can be mounted to a wall. The dispenser may dispense rolled sheet material for a user to dry an object or in a commercial setting, dispense material for wrapping products such as packages, meat, etc. where rolled material is needed; or can be used to dispense liquids or fluid materials such as liquid soap, hand sanitizer, shampoo, etc. The dispenser housing can further include a housing 16 that can include or can be formed from an antimicrobial plastic or composite material or can be formed from a metal or composite material treated with an antimicrobial coating on all touchable surfaces.

In one aspect, as shown in FIGS. 1A-2G, the dispenser 10 can be configured to dispense various types of sheet materials 12 including paper sheet materials such as towels, tissue, napkins, etc. In embodiments, the dispenser 10 generally will include a dispensing mechanism 15 including a driven roller drive assembly 14 mounted or otherwise disposed within a dispenser housing 16 and operable to dispense prescribed amounts/lengths of sheet material. For example, upon activating the dispenser 10, the feed roller drive assembly 14 is engaged and operates to drive or cause rotation of a drive spindle or feed roller 18 (FIG. 1C). In addition, in some embodiments, the dispensing mechanism 15 can be incorporated into a housing or a cassette 17 that can be replaceable mounted within the dispenser housing.

FIGS. 1A-1C show an example embodiment of a sheet material dispenser 10 configured to dispense a sheet material 12 from a single supply 20 of sheet material 12. For example, the dispenser of FIGS. 1A-1C can be configured to dispense tissue paper from a supply roll 20 of tissue paper. In other embodiments, such as shown in FIGS. 2B, 2C, and FIGS. 2E-2G, the dispenser 10 can be configured to dispense sheet materials 12 from two or more supply rolls 20.

It further should be appreciated that the dispenser 10 described herein should not be considered to be limited to any particular style, configuration, or intended use, or to a particular type of sheet material. For example, the dispenser 10 may be operable to dispense paper towels, toilet tissue, or other similar paper or sheet materials, including dispensing or feeding non-perforated and/or perforated sheet materials. In addition, the dispensers and dispensing system can be used in a variety of different environments, including, without limitation, use in a residential setting (e.g., in a bathroom in a person’s home), as well as in commercial locations (e.g., in bathrooms and/or kitchens of restaurants, large venues such as arenas, airports, etc.., and other locations such as in hospitals, schools, etc.).

In embodiments, the feed roller drive assembly 14 of the dispensing mechanism 15 of the dispenser 10 can include at least one feed roller 18 and one or more pressing rolls 19. The rotation of the feed roller 18 in turn pulls the sheet material 12 from a supply of sheet material supply 20 for feeding a predetermined, prescribed, measured or selected amount or length L (e.g., a 10″-12″ or other desired length) of the sheet material 12 along a conveying or feed path P (FIG. 1B) from the roll or the sheet material supply 20 of the sheet material 12 through and out of a discharge, such as a discharge chute 22 or other suitable opening provided/defined in the dispenser housing 16 of the dispenser 10, as is generally indicated in FIG. 1C.

The feed roller drive assembly 14 can be activated and driven/rotated to pull and feed the sheet material12 from the sheet material supply 20 to and through the discharge chute 22 under control of a control system 506 of the dispenser 10. Example embodiments of control systems 506 for the dispensers 10 of the dispensing system 500, which will be configured for operating their dispensers 10, and in some embodiments, can be configured to communicate with and control operation of multiple dispensers of a dispensing system, are generally shown in FIGS. 7A-7D.

For example, in embodiments such as shown in FIGS. 7A-7C, each dispenser can include a control system 506 including a controller 514 that can comprise one or more processors, such as a microprocessor, CPU, etc., a memory 516, and computer programming stored in the memory 516 and executed by the controller 514 for control of the feed roller drive assembly 14 to feed the selected or desired length of sheet material and to monitor the dispenser 10 and components such as the supply of sheet material and usage/operation of the dispenser. The control system 506 further will be in communication with, and will receive a plurality of signals, from at least one activation sensor 414 of an activation system 400, which, in embodiments, can comprise at least one activation sensor or an array or series of activation sensors configured to detect the presence of an individual or user within a selected activation zone, proximity of range and generate an activation signal or prompt to initiate dispensing of the sheet material or other material being dispensed.

The activation system 400 can include an activation sensor assembly 401, which, in embodiments, can include one or more activation sensors 414, which, in various embodiments, can comprise various type sensors or detectors, for example, including an adjustable proximity sensor that can be configured/adjusted to detect the presence of a user's hand or other object at a desired range/location and dispense measured/selected amounts of the sheet material. The proximity sensor can be manually or automatically adjustable. In addition, or in the alternative, one or more pairs of IR sensors (e.g., an emitter and a corresponding detector) that are arranged about/within the discharge chute 22 and transmit/receive signals across the discharge path P to sense or detect the presence or absence of sheet material or other object within the discharge chute or otherwise along the feed path.

In embodiments, any suitable sensor, however, such as a photoelectric, light curtain, or other similar sensing systems/detectors, can be used to detect the presence of a user's hands or other object placed along the dispenser housing 16, and/or the feeding of a selected amount of the sheet material 12 can be used, without departing from the present disclosure. In addition, various sensor arrays and/or control systems can be used.

As indicated in FIGS. 1A and 1C, the dispenser housing 16 includes a roll support mechanism 30, for holding at least one of a roll 32 of the sheet material supply 20 of the sheet material 12. The roll support mechanism 30 can include a pair of supports or arms 34 coupled to the dispenser housing 16 and supporting the roll of sheet material 12, such as indicated at 36. These support arms 34 may be fixedly arranged to hold the sheet material supply 20 of sheet material in a spaced relationship with respect to the feed roller 18. For example, the support arms 34 can be attached or coupled to the dispenser housing 16 by sliding or snap-fitting at least a portion of the supports/arms within grooves or slots 37 defined along a rear portion 36 of the dispenser housing 16. However, in other embodiments, the support arms 34 further can be connected to the dispenser housing 16 in any suitable manner, such as with one or more fasteners or other suitable connection mechanisms. As a further alternative, the support arms also can be integrally formed with the housing without departing from the present disclosure. In additional or alternative constructions, the support arms 34 also may be biased or urged, such as by a spring or other suitable biasing mechanism(s), or by a general resiliency, toward the feed roller 18 to urge or direct the sheet material supply 20 of sheet material downwardly toward or against the feed roller 18.

In addition, the feed roller 18 will be movably or rotatably mounted within the dispenser, for example, being rotatably coupled to one or more walls or other portions of the dispenser housing 16. For example, the ends of the feed roller 18 can be connected, mounted, or otherwise coupled to the dispenser housing 16 by one or more bearing assemblies and/or other suitable support mechanisms that support and allow for rotation of the feed roller 18 in relation to the dispenser housing 16.

As illustrated in FIG. 1B, the dispenser 10 further generally can include one or more pressing roller(s) 60. The pressing roller(s) 60 can be biased toward engagement with the feed roller 18, so as to engage and urge or press the sheet material against the feed roller 18 with a force sufficient to draw or pull the sheet material therebetween upon rotation of the feed roller 18. The pressing roller(s) 60 can be mounted within the dispenser housing 16, such as with the ends thereof held within one or more arms or supports of a bracket 60A in a manner to enable rotation of the pressing roller(s) 60. The bracket 60A also can be biased by a biasing member, such as a spring, so that the pressing roller(s) 60 can be urged toward the driven feed roller 18.

Additionally, or in the alternative, one or more of the pressing roller(s) 60 further can be disposed within a frame or other structure and biased toward the feed roller 18 such as by compressing/tension springs or other suitable springs, biased cylinders, or other biasing mechanisms. In one construction, the frame can support at least two pressing rollers and also can be pivotable to enable one pressing roller to move away from the feed roller as needed, while the other roller is pivoted into closer contact with the feed roller (not shown). In addition, or alternatively, in some embodiments, the pressing roller(s) 60 may be driven by drive mechanism, for example, off of the motor that drives the feed roller or by a separate drive, so as to help facilitate feeding of the sheet material 12.

The feed roller drive assembly 14 includes at least one driving mechanism, e.g., a motor 74, that is in communication with the feed roller 18 so as to drive movement/rotation thereof (FIG. 1C). The motor 74 can include a brushless servo or stepper motor or other, similar type of variable speed electric motor, and communicates with the control system 506 of the dispenser 10 to receive instructions and power for activating and driving the feed roller 18 through a dispensing cycle (e.g., a determined time, number of revolutions, etc.), so as to feed the selected or desired amount/length of the sheet material through the discharge chute 22 of the dispenser 10.

In one additional aspect, the drive system/assembly 14 also can include a transmission assembly 76 for transferring power between the motor 74 and the feed roller 18. For example, the transmission assembly 76 can include a drive belt 78 and/or drive gears coupling the motor 74 to the feed roller 18. In alternative constructions, the feed roller drive assembly 14 can include a gear assembly including a plurality of intermeshing gears that operatively connect the motor 74 and the feed roller 18. Any suitable transmission mechanisms, device, assemblies, etc. can be used for transferring power between the driving mechanism and the feed roller, without departing from the scope of the present disclosure.

In addition, FIGS. 2A-2G illustrate an embodiment of a dispenser 10, which can be configured for dispensing sheet materials from multiple supplies 20 of a sheet material 12. The dispenser 10 of the FIGS. 2A–2G can have a substantially similar construction to the dispenser of FIGS. 1A–1C, including a housing 16 within which multiple (e.g., two) supplies 20 of sheet material 12 are received. For example, an embodiment, the dispenser 10 of FIGS. 2A–2G can be used to feed tissue, toilet paper, or other, similar sheet materials, with each roll of sheet material being supported by a roll support mechanism 30. As indicated in FIG. 2C, an embodiment, the roll support mechanism can include a pair of supports or arms 34 can be pivotally attached to a portion of the housing 16, for example, being pivotally attached to a cover portion 16A of the housing. As indicated in FIG. 2C, the rolls of sheet material can be received on the support arms 34 when the support arms are in a raised or pivoted up configuration and then can be lowered into a feeding configuration with a portion and of the sheet material of each roll of sheet material being engaged with the dispensing mechanism 15 as shown in FIG. 2B.

In embodiments, the dispensing mechanism 15 of the dispenser of FIGS. 2A-2G can include a pair of feed rollers 18, sets of pressing rollers 60 and a feed roller drive assembly 14 that can be coupled to each of the feed rollers 18 or selectively driving each of the feed rollers as needed for feeding for selectively feeding the sheet material from one or the other of the supply rolls. For example, in embodiments, the feed roller drive assembly 14 can include at least one driving mechanism such as a motor 74 that can be linked in communication with each of the feed rollers 18. The driving mechanism can be in communication with the control system 506 of the dispenser to receive control instructions and power for activating and driving a selected one of the rollers 18 during a dispensing operation or cycle (e.g., a selected time, number of revolutions, etc.), so as to feed the selected or desired amount/length of the sheet material through the discharge chute 22 of the dispenser 10.

According to some aspects, the feed roller drive assembly 14 can have a similar construction as shown FIG. 1C, including, in embodiments, a transmission assembly 76 for transferring power between the motor 74 and the feed rollers 18. For example, the transmission assembly 76 can include a drive belt 78 and a drive gear assembly that can include a series of drive gears coupling the motor 74 to the feed roller 18. In addition, a gear clutch or one-way bearing assembly 80 can be provided, for example, being positioned between the drive gears linked to each of the feed rollers and with the drive belt being extended thereabout. In embodiments, the drive motor can be a reversible motor and can be selectively driven by the control system in a first direction to feed the sheet material from a first one of the supply rolls, and in a second direction to feed the sheet material from the second supply roll (e.g., when the first supply roll has been exhausted or depleted). When the driving direction of the motor is switched, the gear clutch or one-way bearing assembly can cause the drive belt to be disengaged from the drive gear associated with one of the supply rolls (e.g., the first supply roll) and engaged with the drive gear of the other supply roll (e.g., the second supply roll) to selectively switch feeding between the supply rolls as needed. Any suitable transmission mechanisms, device, assemblies, etc. can be used for transferring power between the driving mechanism and the feed roller, without departing from the scope of the present disclosure.

Still further, is some other embodiments, a pair of drive motors could be provided, each linked to an associated one of the feel rollers. In embodiments, the drive motors can be selectively operable by the control system for selectively feeding sheet material from one or the other of the supplies of sheet material.

In addition, in embodiments of the dispensers such as shown in FIGS. 1A-2G, one or more paper detection sensors 608 can be positioned along/adjacent the discharge of the dispenser, for example, being located within a throat portion of the discharge, while in other embodiments, the paper detection sensor (s) can be positioned above the throat or below the outlet of the discharge. In embodiments, the paper detection sensor(s) 608 can comprise a signal device such as a proximity sensor switch or the like adapted to detect the presence or absence of paper within the discharge or throat and send a signal to notify the controller of the control system of the dispenser that the sheet material has or has not been removed once the dispensing operation has ended. By way of example, the paper detection sensor(s) can include an infrared emitter and detector that can be adapted to detect the presence or absence of the sheet material, though any suitable sensor can be employed such as a proximity sensor or other detector, a magnetic switch, or a mechanical switch. After receiving a signal indicating removal of the sheet material, the control system 506 further can reset the dispensing mechanism for a next dispensing operation of cycle.

In addition, in embodiments, the dispenser also could include a pawl member or additional paper sensor configured to indicate the removal of the sheet material from the dispenser. In such embodiments, the pawl member can be positioned to be engaged by the sheet material as a user pulls/removes the paper from the dispenser, and when released, can indicate the sheet material has been removed, and if not released or returned to a home position when the dispensing operation is completed, a signal can be sent to the controller to indicate the sheet material has not been removed.

Still further, in some constructions, the dispenser can include a cutting mechanism configured to move or be actuated at a prescribed or preset point during a revolution of the feed roller 18, or after a prescribed rotation of the feed roller 18 so as to selectively cut or perforate the sheet material after a desired or prescribed length or portion of the sheet material has been fed or dispensed. In embodiments, the dispenser may include one or more suitable movable cutting members or can include a stationary blade or tear bar or other cutting member disposed adjacent or along the dispenser housing 16 so that a user can separate a sheet or measured amount of the material by grasping and pulling the sheet across the stationary tear bar.

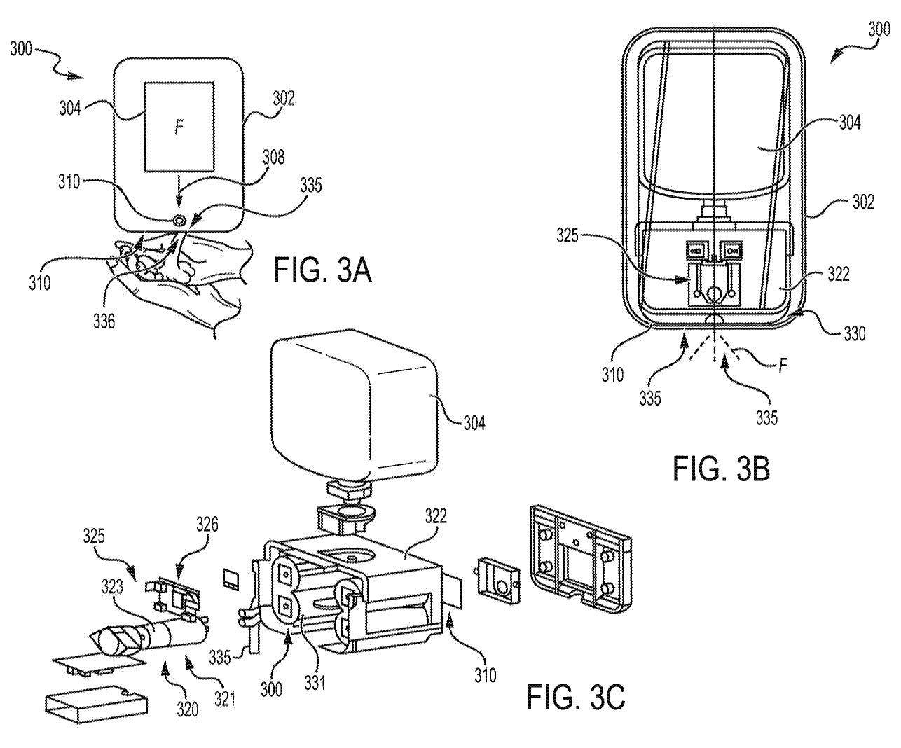

FIGS. 3A-3C illustrate an additional example embodiment of a dispenser 300 that can be part of the dispensing system, which dispenser comprises a liquid or fluid dispenser such as soaps, sanitizers, shampoos and/or conditioners, lotions, and other fluids. In addition, it will be understood that the terms “fluid” and “liquid” can refer to any type of fluid media and are not to be construed as limited to particular fluids or liquids.

In embodiments, the fluid dispenser 300 generally includes a dispenser housing 302 that supports/houses a supply or reservoir 304 that contains/stores a liquid, e.g., liquid soap, hand sanitizer, etc. For example, in embodiments, such as shown in FIGS. 3B-3C, the dispenser 10 generally will include at least one supply 304 of a fluid or liquid “F”, which can comprise a bottle, bag, or other, similar container defining a supply chamber configured to hold a selected volume of the fluid material. For example, in embodiments, the fluid supply can contain a selected or predetermined volume of fluid that can provide a preferred or selected number of fluid dispenses (e.g., the supply can include a container with approximately 1000ml to 2000ml, or other volumes as needed). Still further, in some embodiments, the supply 304 can be configured to be refillable, while in other embodiments, the supply can be provided as a single use container that can be recycled once empty to help protect against contamination of the fluid therein.

As further illustrated in FIG. 3C, in embodiments, the dispenser 300 further will include a dispensing assembly 320 including a dispensing mechanism 321 in communication with the fluid supply 304 and configured to selectively draw and dispense prescribed amounts or “shots” of the fluid from the fluid supply, and a control system 325. In embodiments, the control system 325 can comprise a controller 326 that can include one or more processors, and, in embodiments, can be contained within a housing or cassette 322 within which the dispensing mechanism 321 is housed, or, in other embodiments, can be separate from the dispensing assembly and dispensing mechanism.

In embodiments, the dispenser 300 can include an internal power supply 330, such as a series of batteries 331, while in other embodiments, the dispenser could be coupled to an external power source, such as a building power supply, external batteries, or other sources. In embodiments, the controller will include programming or otherwise will be configured to initiate and control operation of the dispensing mechanism to perform a dispensing operation to dispense the fluid.

In embodiments, the dispensing mechanism 321 can include at least one pump 323 that will be driven by a motor or other actuator to draw an amount of fluid from the fluid supply 304 and direct the fluid to a discharge for the dispenser. In addition, in embodiments, the fluid dispenser 300 further can include one or more nozzles 335 located at a discharge outlet 336 for providing the fluid to the user. In some embodiments, the nozzles can be configured to cause the fluid to be dispensed as a foam, while in other embodiments, the fluid can be dispensed in a substantially liquid or gel form.

The fluid dispenser 300 further can include one or more activation sensors 310 or an array of activation sensors, e.g., including an IR sensor, proximity sensor or other detector configured to detect the presence of an individual within a selected activation zone, proximity or range adjacent the dispenser. The one or more activation sensors generally will be in communication with the control system 325 of the dispenser, so as to communicate/transmit an activation signal or prompt to the controller 326 upon detection of a user within the selected activation zone, proximity of range, in response to which the controller can control operation of the pumping mechanism 308 to initiate and conduct a dispensing operation.

In embodiments, the one or more activation sensors 310 can be configured as proximity sensors to gather information that is related to the presence of an object, such as a user's hand, near or proximate the fluid dispenser 300. Accordingly, in operation, when a user places their hand in proximity to the one or more sensors 310, the one or more sensors 310 provide a signal to the controller to activate the pumping mechanism 308 for dispensing a select or prescribed amount of liquid. Still further, in embodiments, the control system, including the controller and at least one activation sensor, and the dispensing mechanism and other operative components of the dispenser can be in communication with a power management system that further is coupled to the power source, e.g., one or more batteries or an AC power source, and will be operable to control the supply of power to the controller 326, at least one activation sensor 310, dispensing mechanism 308, and other operative components to minimize power usage by the dispenser, as discussed further below.

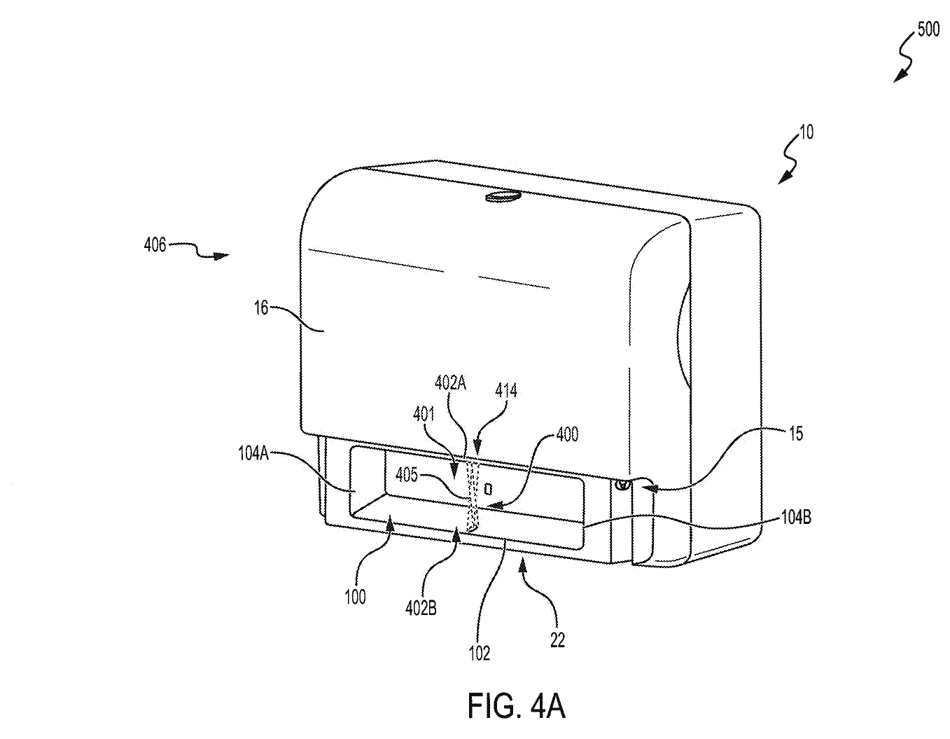

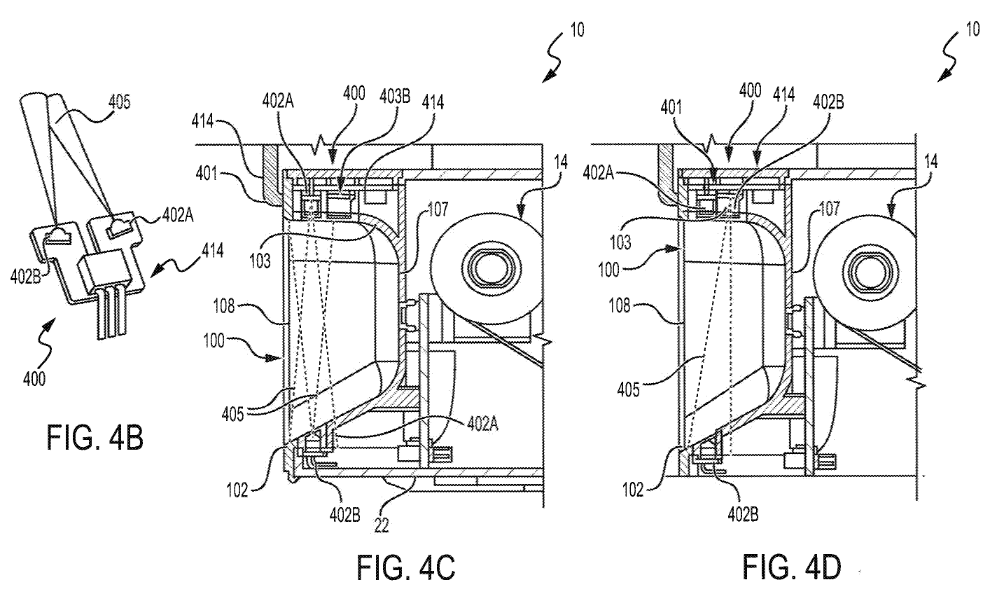

FIGS. 4A-6D illustrate example embodiments of dispensers with embodiments of activation sensor assemblies 401 of the activation system 400 of the dispensers that can be located in various locations along portions of the dispensers and which can provide varying activation zones, proximities or ranges for the capture of a user (e.g., capture of infrared radiation from the hand or other part of a user) to generate an activation signal or prompt to initiate a dispensing operation. FIG. 4A shows an example embodiment of a dispenser 10, which is shown as similar to the embodiment of a dispenser shown in FIGS. 2A-2G, generally including a dispenser housing that can support/house one or more supplies of a material to be dispensed, e.g., paper towel, hand towel, fluid containers, etc. The dispenser further can include a discharge outlet through which the sheet material can be dispensed from the dispenser housing to the user, and a dispensing mechanism (FIGS. 4C-4D), for directing or moving the material to be dispensed from the supply to the discharge outlet.

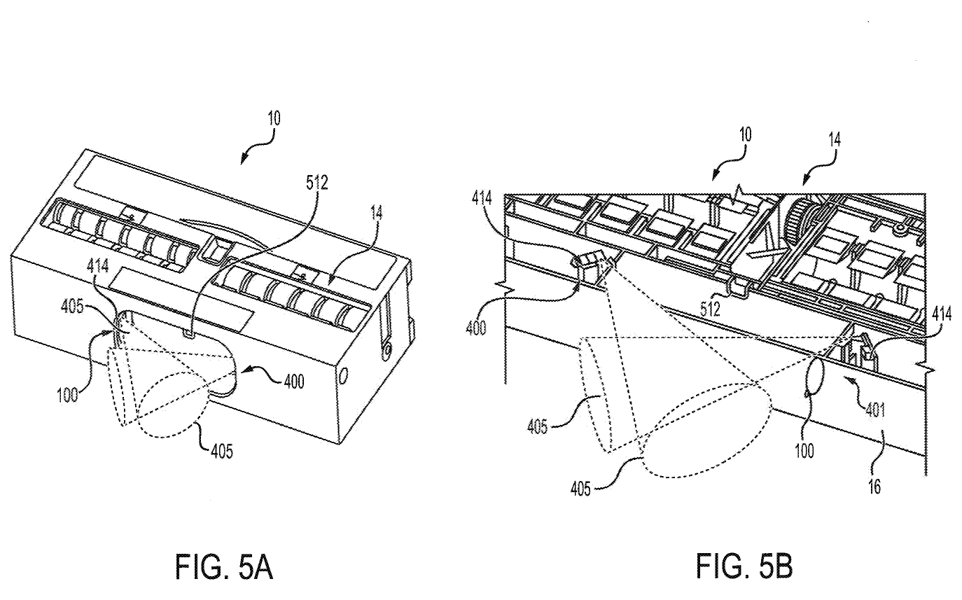

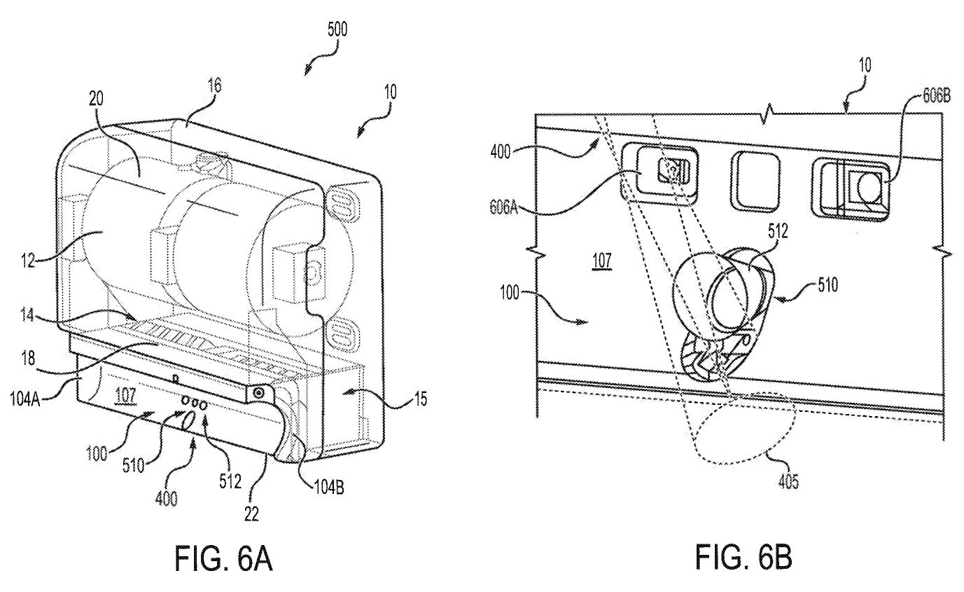

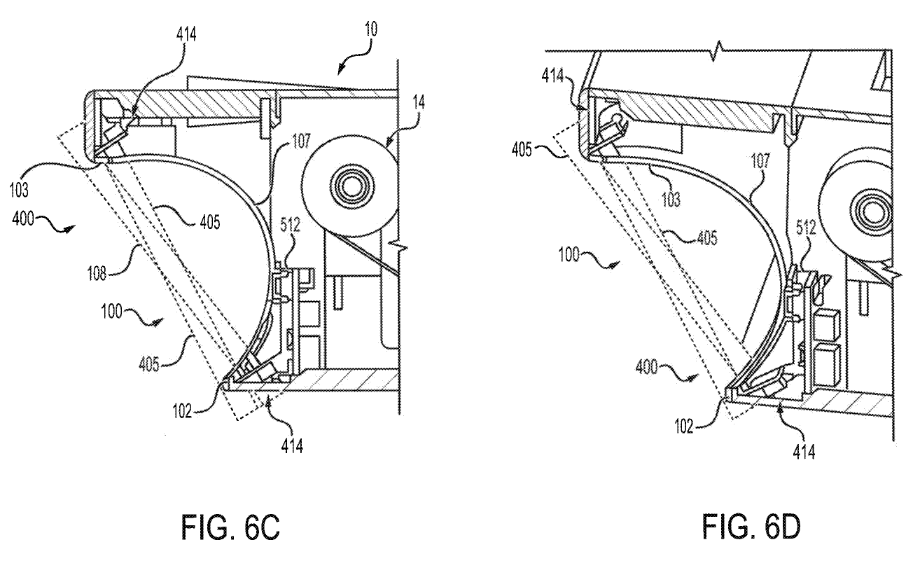

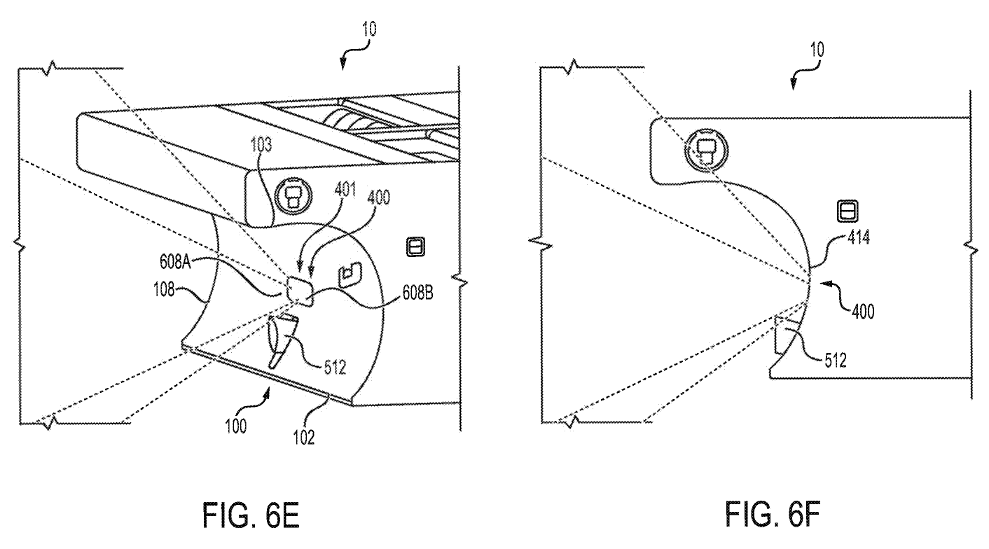

In embodiments, as shown in FIGS. 2A, 4A, 5A, and FIGS. 6A-6F, the dispenser 10 can include a recess or pocket 100 in which an activation system 400 can be located. As indicated in FIGS. 4A-6F, the pocket or recess of the dispenser can have various configurations. For example, in FIGS. 2A and 4A-4D, the pocket or recess can have a generally elongated substantially rectangular construction, while in FIGS. 5A-5B, the pocket or recess can have a smaller area. FIG. 6A shows another embodiment in which the pocket or recess is formed without a bottom or lower edge, and which also may be configured with open sides.

In embodiments, the activation system 400 may include at least one activation sensor 414 e.g., including an IR sensor, proximity sensor or other, similar sensor in communication with a controller 510 of a control system 506 of the dispenser/dispensing system and with a power management system 510. As shown in FIG. 7C, the power management system will be configured to selectively control the flow/transmission and use of power by the operative components of the dispenser, including the controller, the dispensing mechanism, the one or more activation sensors of the activation system 400, and other potential operative components (e.g., one or more transmitters, status indicators, lights, dispenser controls such as resets, etc..). In embodiments, the power management system can be configured to separately and independently connect or disconnect the operative components and the power supply. For example, in embodiments, the power management system can be configured to connect only the one or more sensors of the activation system upon detection of infrared radiation withing the prescribed detection area, zone, or range while maintaining the other components in a powered-off or decoupled state; while in other embodiments, the power management system can be configured to periodically power on or off the operative components, for example, disconnecting one or more of the components from the power supply after a prescribed time or other event; and/or connecting the control system or a Wi-Fi transmitter of the dispenser to the power supply at intervals to send updated dispenser information including dispenser usage and diagnostic information such as amounts dispensed, times of dispensing, personnel using the dispenser, error conditions such as jams, no supply or low supply or low power.

In embodiments, the activation system 400 can be located within the pocket or recess 100 formed or defined along a portion (e.g., a front or lower forward edge) of the housing 16 of the dispenser, such that at least one activation sensor 414 (e.g., a hand sensor) thereof can be located within a generally protected area. The at least one activation sensor can be arranged/oriented within the pocket or recess for sensing the presence of a user or a portion thereof within a selected activation zone, proximity or range, for example, when the user places their hand within the pocket or recess 100. The location of the at least one activation sensor within the pocket or recess can help define or focus the activation zone, proximity or range and help avoid inadvertent detections that could initiate an unwanted dispensing operation. In response to detection of a user by the at least one activation sensor 414, the controller 506 may determine which of the plurality of supplies 404 for the dispensing mechanism 408 to activate and start a dispensing cycle or operation for dispensing sheet material from the selected supply of sheet material.

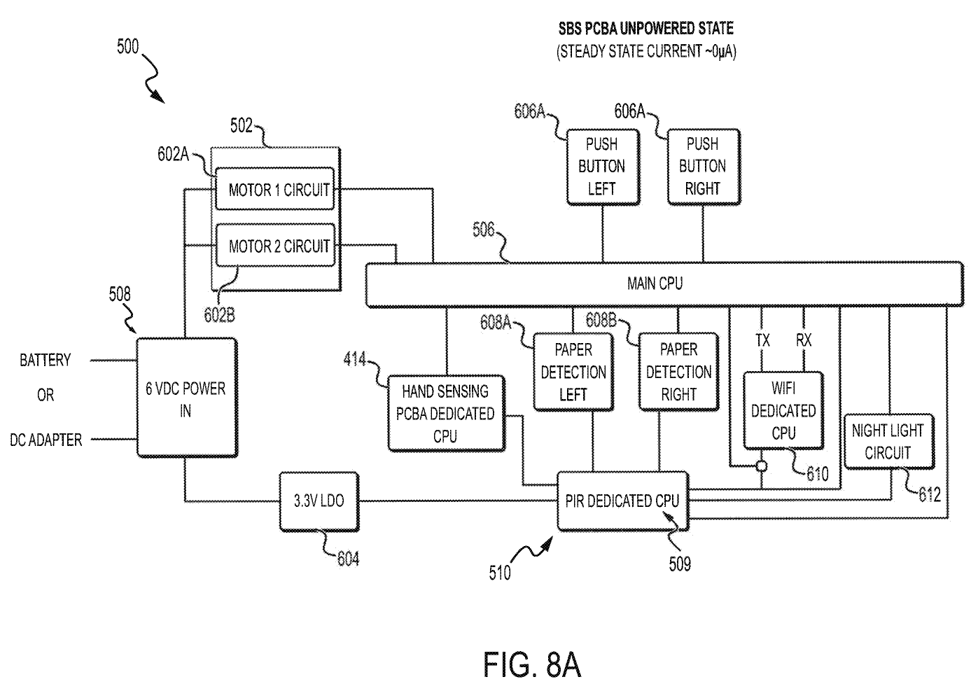

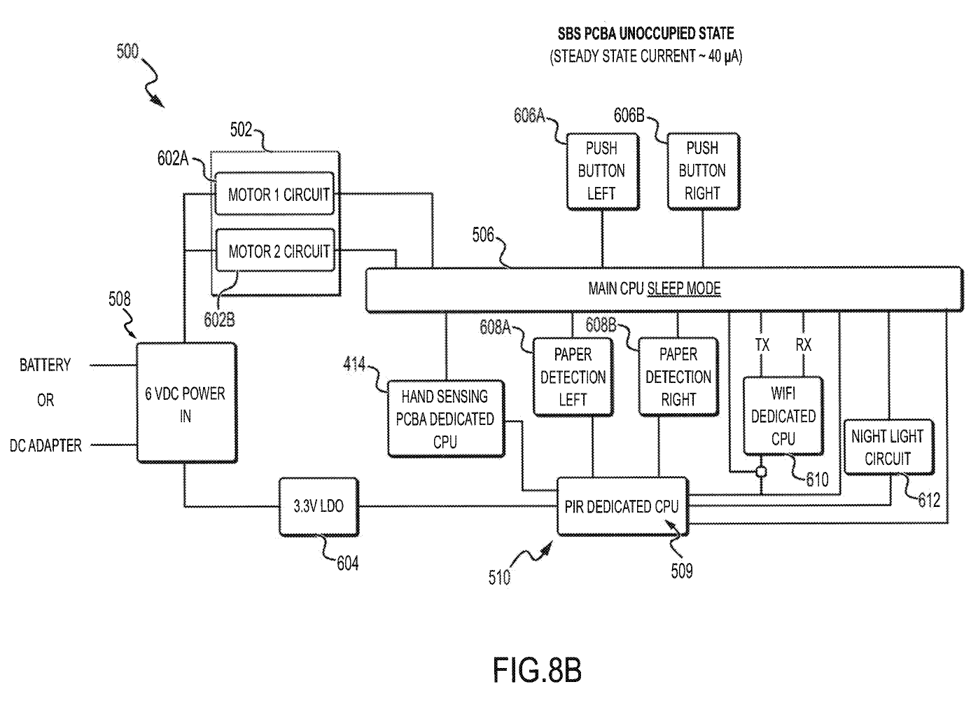

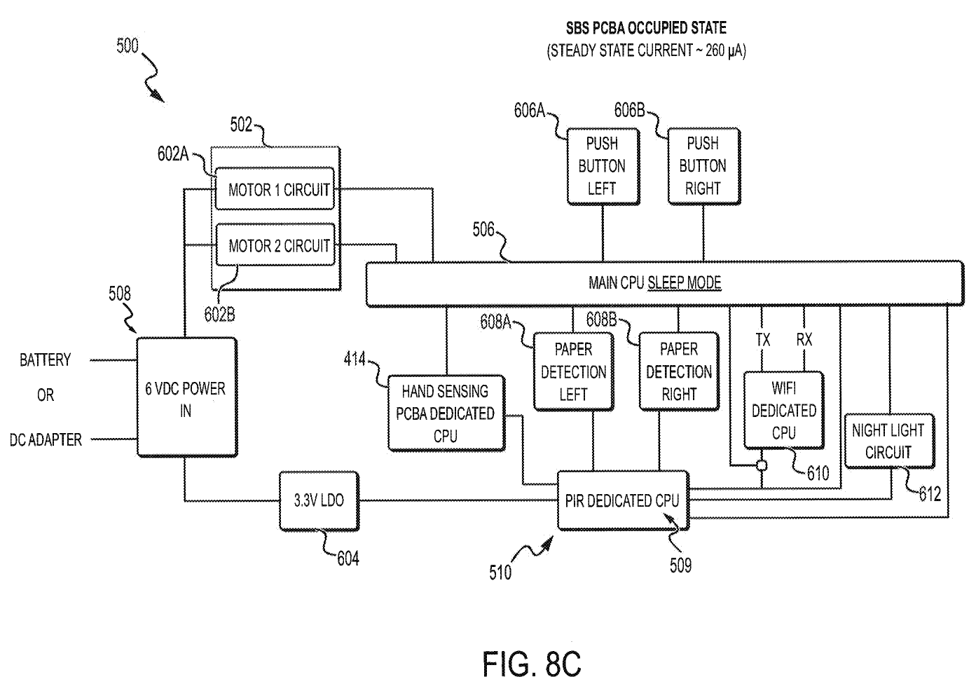

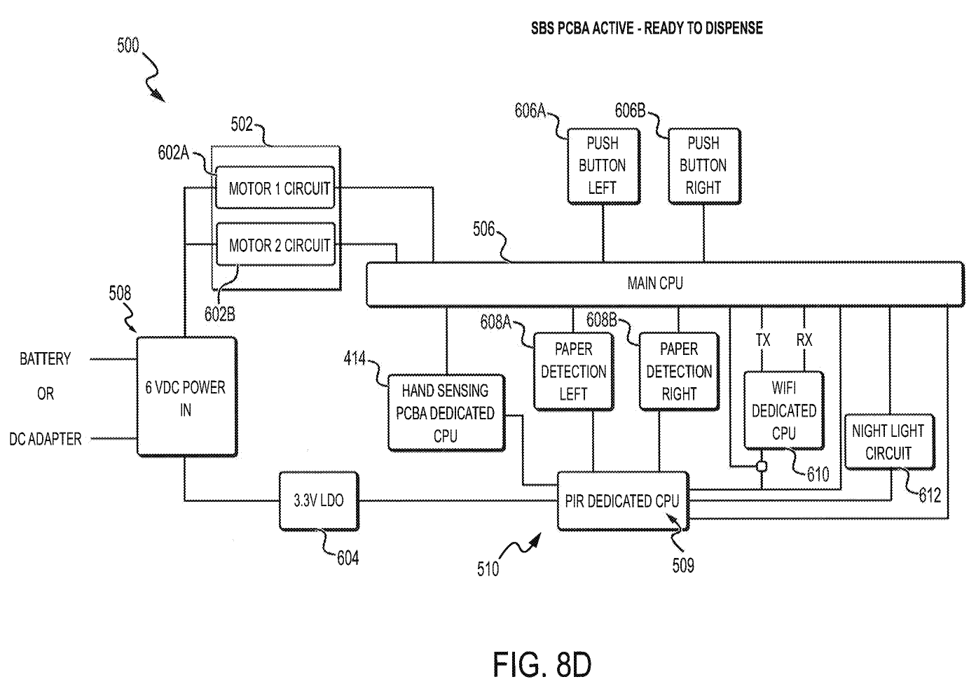

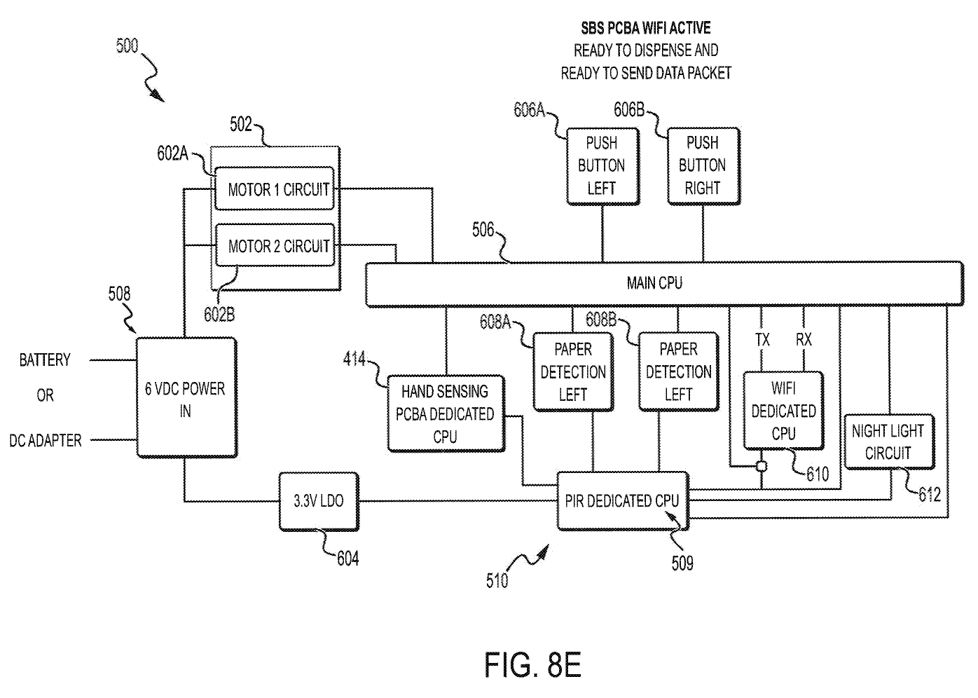

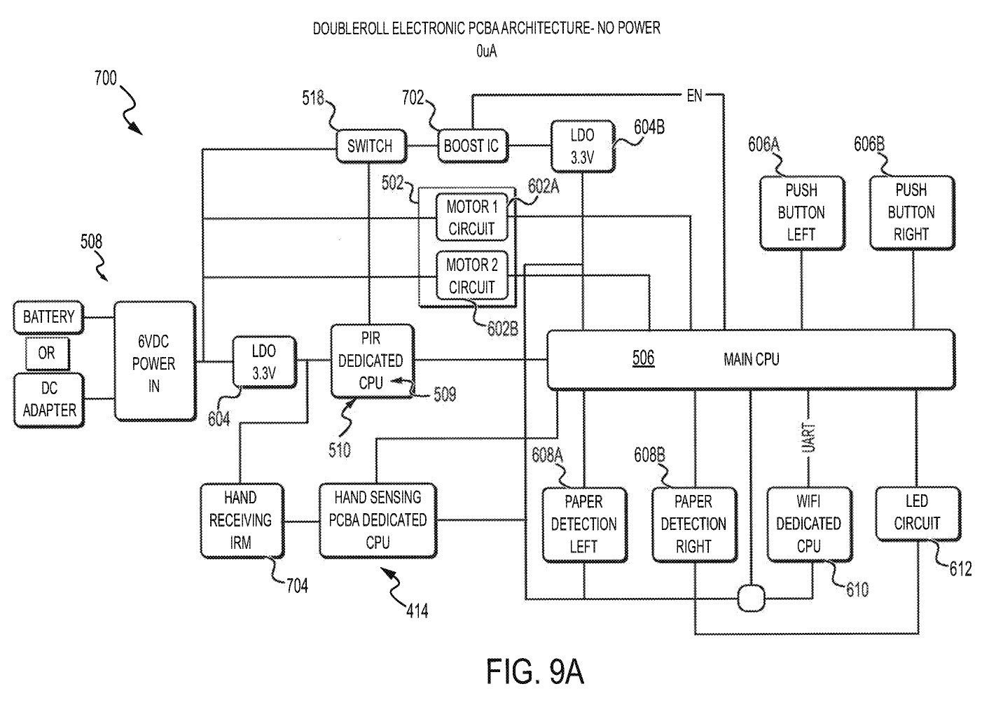

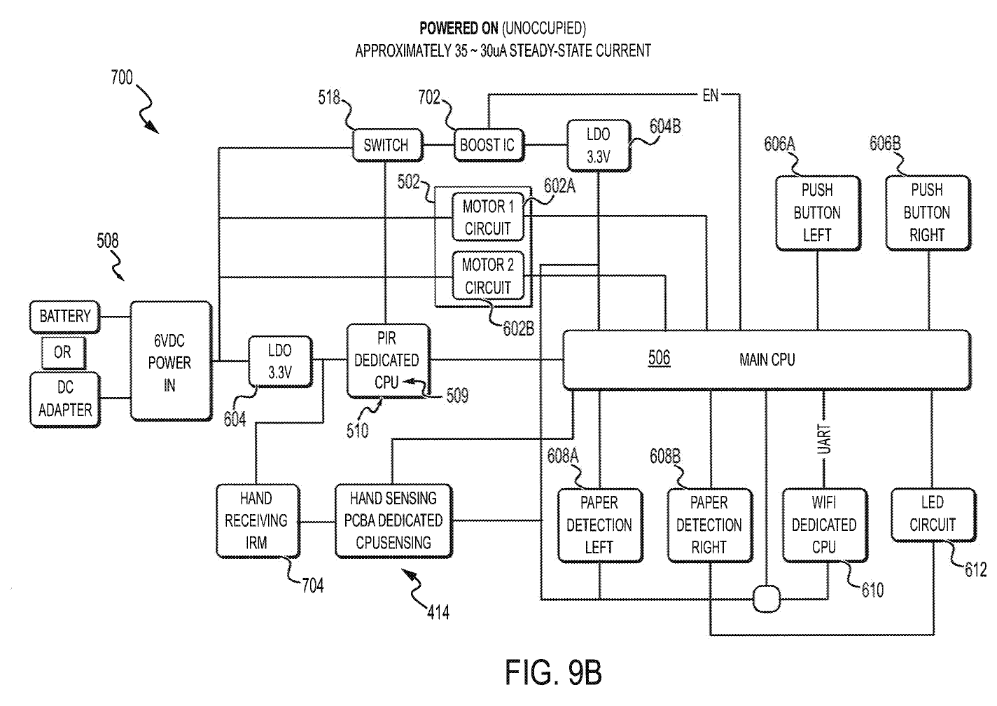

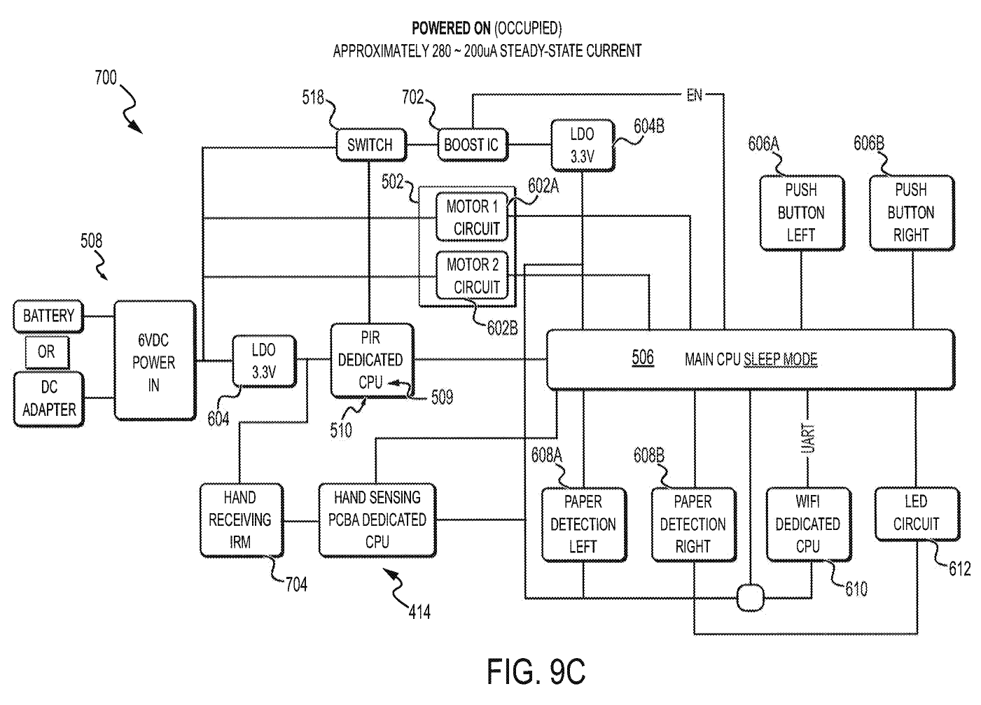

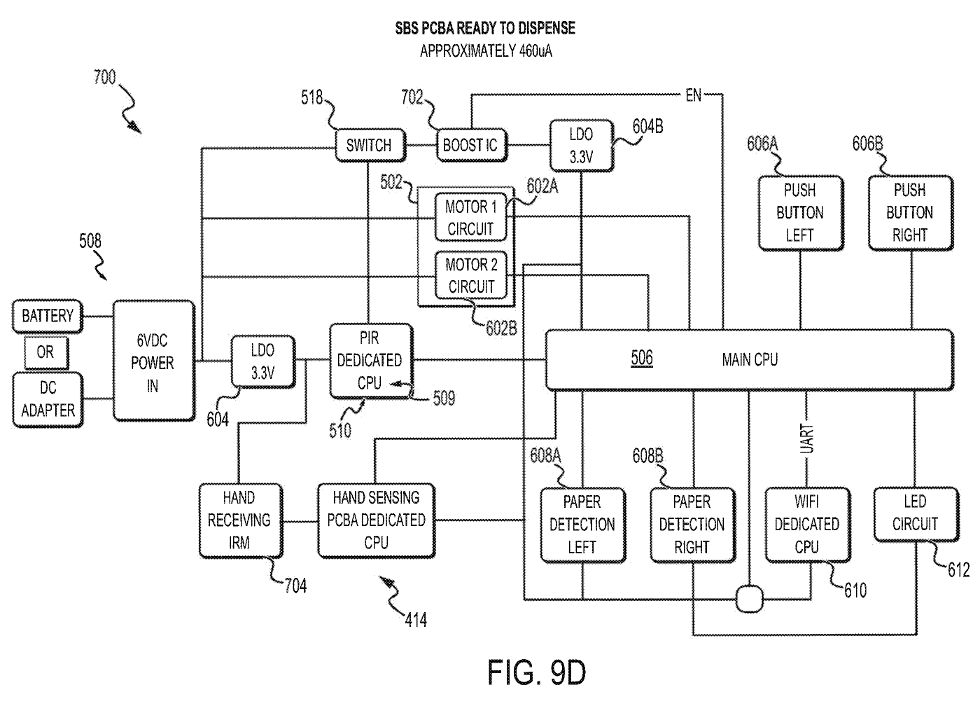

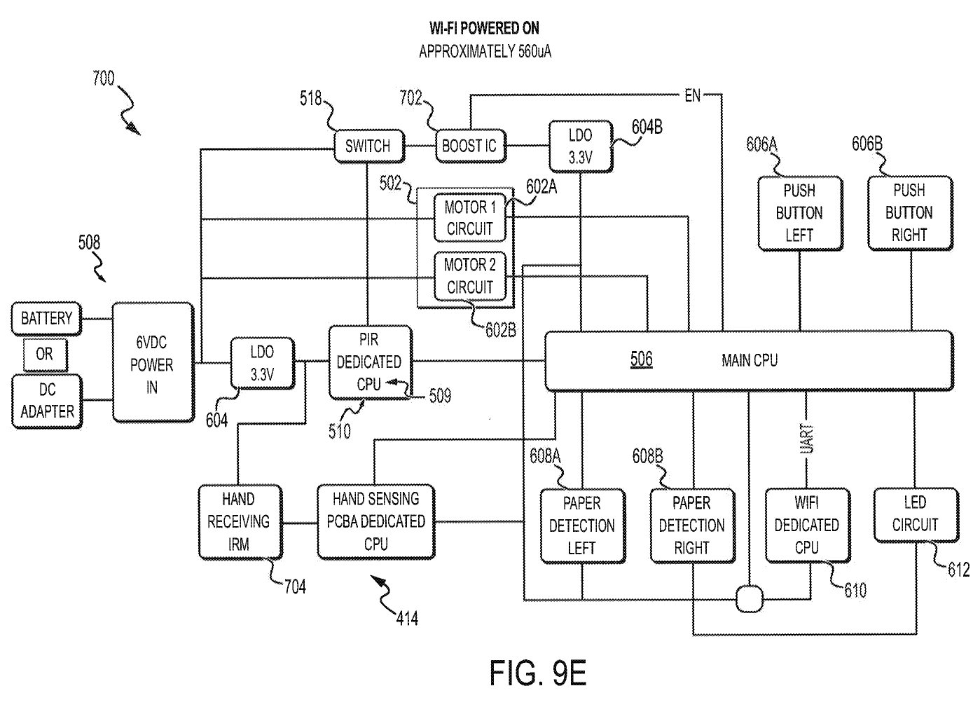

In embodiments, the activation sensor 414 of the activation system 400 can be configured as a proximity sensor with a focused/selected activation zone, proximity, range or area 405 for detection of the presence of an object, such as a user's hand, near or proximate the dispenser 400, such as, for example, detecting the user’s hand when it is placed in the pocket or recess 100, but not when it is simply near the dispenser, to help reduce activations or the dispenser being fully powered up and in a “powered on,” ready to dispense state such as shown in FIGS. 8D-8E and 9C-9E. FIGS. 2B, and FIGS. 4A-6F illustrate various example embodiments or arrangements of activation sensors, activation sensor arrays, and different configurations of activation zones for the activation systems 400 according to principles of the present disclosure.

For example, one non-limiting example embodiment sensor that could be used as an activation sensor 414 of the activation system 400 is generally shown in FIG. 4B and can include an infrared emitter 402A and an infrared receiver or detector 402A, which can be configured to emit an IR beam (indicated by 403). Other types or configurations of sensors also can be used.

In other embodiments such as illustrated in FIGS. 4A, 4B-4C, 5A-5B, 6A, and FIGS. 6C-6F, the activation system 400 can include a variety of types and arrangements of activation sensors 414. For example, FIGS. 4A and 4C-4D show arrangement of activation sensors including one or more emitters 402A and one or more receivers 402B that can be located along an upper portion or surface 101 and a lower portion or surface 102 of the pocket or recess 100. The emitters and receivers can be arranged in an opposing relationship, with one or more IR beams being transmitted therebetween. The transmission of the IR beams can be varied so as to provide different activation zone configurations as indicated at 405.

In addition, in other embodiments such as shown in FIGS. 5A-5B, a pair of activation sensors can be positioned along opposite sides 104A/104B of the pocket or recess 100 and can project generally conically shaped sensing beams that can cross to define an activation zone with overlapping fields of coverage.

FIGS. 6A-6F show still further arrangements of activation sensors 414 and sensor arrays 401, which can define/create still other activation zone configurations and can extend in varying directions, including forwardly, towards the sides of the dispenser, downwardly below the bottom of the dispenser (e.g., where the pocket or recess does not include a bottom portion), or combinations thereof. As also shown in FIGS. 6E-6F, in embodiments, an activation sensor array, which can include one or more activation sensors 414 positioned along a rear portion 107 of the pocket or recess, adjacent the PIR sensor of the power management system of the dispenser, and which can project a sensing beam forwardly out to the open front portion 108 and, in embodiments, at least partially downwardly and/or to the sides.

Accordingly, in operation, in embodiments, when a user places their hand in proximity to the one or more activation sensors 414, the activation system 400 provides a signal to the controller of the dispenser to activate the dispensing mechanism for dispensing the sheet material. The controller further is coupled to a power source, e.g., one or more batteries or an AC power source (e.g., the dispenser could include a plug-in type of connector for connecting to a building power supply), such as the power source 508 in FIG. 7A to power the controller, the dispensing mechanism 408, and the activation system 400.

In an embodiment, after receiving a signal indicating removal of the sheet material, the controller further can activate a paper detection sensor 416 to verify that the sheet material has been removed from the discharge chute.

FIG. 7A-7D shows an exemplary embodiment of the dispensing system 500 for at least one dispenser as previously described. The elements of FIG. 7A-7D are herein described in relation to elements of the previous figures. In FIG. 7A, the dispensing system 500 can include at least one dispenser including a dispensing assembly 501, which, in embodiments, can comprise a dispensing mechanism 502, at least one activation sensor 414, and a control system 506 including a controller 514 (which can, in embodiments, include one or more processors). The dispensing system 500 further generally will include a power source or supply 508, and a power management system 510 in communication with or coupled to the dispensing assembly 501 and the power source 508. In embodiments, the power management system further will include at least one detection sensor.

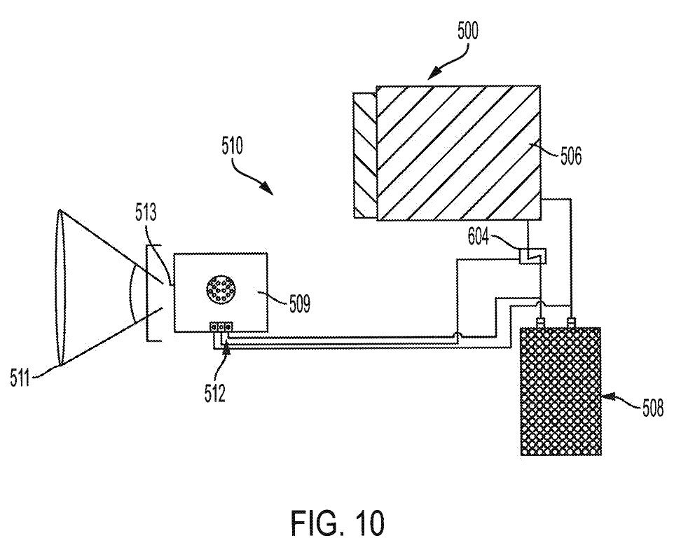

In embodiments, the at least one detection sensor of the power management system can comprise at least one passive infrared radiation (PIR) sensor 512. For example, FIG. 10 illustrates an example embodiment of a PIR sensor 512 that can be used with embodiments of the power management system 510. In embodiments, the PIR sensor 512 can comprise a “smart” PIR sensor including a processor 509 coupled to the power supply and which can communicate with one or more switches 604 to selectively couple or decouple various operative components of the dispenser 10 of a dispensing system 500, including the control system 506, dispensing mechanism, activation sensors, transmitter(s), and/or other operative elements of the dispenser to power 508. In embodiments, the PIR sensor 512 further can include a lens 513 that can be configured to create a selected detection zone, area, range or distance as shown at 511. For example, a generally comical IR transmission, having an angle “α” can be generated, extending for a predetermined distance and/or width. Other configurations also can be provided.

While embodiments of power management system are described as including a detection sensor comprising at least one PIR sensor, it will be understood that other types of detection sensors also can be used. For example, in some environments, the dispenser(s) can be subjected to higher temperatures and/or brighter lighting that, in some cases, require an individual to be closer to the PIR sensor before determining that infrared radiation generated by a person is within the detection range, zone or area. In some such cases, the ability of a PIR sensor to capture/identify the presence of an individual, and further whether the individual has used the dispenser, can be limited, which can potentially affect the tracking of movements of such an individual throughout a facility, and/or tracking compliance of the individual with facility procedures/protocols. In such cases, the at least one detection sensor of the power management system can include at least one radar sensor that can be used in conjunction with or as an alternative to the at least one PIR sensor.

In embodiments, the at least one radar sensor can be configured to send out pulses of radar waves. The pulses of radar waves can be transmitted periodically or on a substantially continuous basis. In embodiments, a time it takes for the radar waves to return after engaging an object can be used to determine movement of an individual within the detection range, zone, or area. By way of example, in some embodiments, if a CPU of the radar sensor determines that the time required for the radar waves to return exceeds a threshold, it can cause the power source to be connected to one or more operative components of the dispenser such as the at least one activation sensor, controller, transmitter, etc. Alternatively, if the CPU detects a difference in timing of the return of the radar waves versus one or more previously returned radar waves, it can initiate such a connection of the one or more operative components to the power source.

In embodiments, the power source or supply 508 can comprise an internal power supply (e.g., one or more batteries or power cells such as shown in FIG. 3D), or can comprise an external power source (e.g., the dispensing system and/or one or more individual dispensers thereof can be coupled to a building power source, battery pack, etc..). In embodiments, the power management system 510 associated with the dispensing system (e.g., with each dispenser or multiple dispensers of the dispensing system also can include or can be in communication with one or more switches 518, which can be integrated with the power management system, or, in some embodiments, can be a separate component(s) positioned between the operative components of an associated dispenser (e.g., the controller of the control system, one or more activation sensors, the dispensing mechanism, indicators, dispenser controls, etc..) and the PIR sensor 512.

FIG. 7B, in an embodiment, shows the dispensing system 500. The dispensing system 500 may include the dispensing assembly 501 in communication with the activation sensor 414. The activation sensor may be in further communication with the switch 518. In embodiments, the switch may be in further communication with the PIR sensor 512 and the power management system 510. The PIR sensor 512 and the power management system 510 may be in further communication with the power source 508.

FIG. 7C, in an embodiment, shows the dispensing system 500 with two switches 518, a first switch 518A and a second switch 518B. The dispensing system 500 may include the dispensing assembly 501 in communication with the second switch 518B. The second switch 518B may be in further communication with the power source 508 and the activation sensor 414. The activation sensor 414 may be in further communication with the first switch 518A. The first switch 518A may be in further communication with the PIR sensor 512 and the power management system 510. The PIR sensor 512 and the power management system 510 may be in further communication with the power source 508.

FIG. 7D shows an example embodiment of a dispensing system 500 according to principles of the present disclosure. The dispensing system 500 generally can include a dominant, primary or lead dispenser 520 and a plurality of secondary, follower or drone dispensers 522 in communication with the lead dispenser 520. The plurality of drone dispensers 522 can include one or more liquid dispensers 522A, such as soap dispensers, hand sanitizer dispensers, etc. and/or one or more sheet material dispensers 522B, such as tissue dispensers, paper towel dispensers, etc. In embodiments, the dispensers 520/522 can include the dispensers 10, 300, or 400. The lead dispenser 520 can include a sheet material dispenser (e.g., tissue dispensers, paper towel dispensers, etc.) or a liquid dispenser (e.g., a soap dispenser or a hand sanitizer dispenser). Each of the dispensers 520/522 can generally include the power management system 510, the activation sensor 414, and the dispensing assembly 501 as shown in FIG. 7A-7C.

In embodiments, the lead and drone dispensers will be configured for bidirectional or cross-communication between each of the drone dispensers and the lead dispenser; and at least some of the drone dispensers also can be configured for cross communication with one or more additional ones of the drone dispensers. In embodiments, each of the dispensers of the dispensing system can be configured to send dispenser usage and diagnostic information at least to the lead dispenser, though in some cases such usage and/or dispenser diagnostic information can be shared between one or more drone dispensers as well. In embodiments, such bidirectional communications can be transmitted/exchanged either periodically or in response to a request from the lead dispenser, which requests can, in embodiments, include instructions from the lead dispenser, such as for a reset of a drone dispenser or for the collection of requested diagnostic information such as, for example, low supply levels (including whether one supply of a series of supplies of sheet material or fluid in the dispenser, or a supply of any of a series of dispensers in the area, is low or empty), fault conditions (e.g., low power, jams or disruptions in operation of the dispenser(s), and usage of a particular dispenser by personnel (which can be used for compliance tracking), etc.

This communication between dispensers enables enhanced monitoring and generation of dispenser diagnostic information needed from a maintenance or servicing perspective, which can be developed and communicated to or accessed by authorized personnel in substantially real time or with minimal delay. Such cross communication between dispensers further enables enhanced power management of the dispenser operations, for example, enabling selective shut down and powering of various dispensers within a same area (e.g., within a bathroom, dispensers in one or more stalls can be shut down while though adjacent a sink can be turned on or remain powered on). As a result, the dispensing systems can maximize the monitoring, collection of information, and the total amount and types of information collected and made accessible to management/supervisory personnel to provide increased efficiencies in operation, substantial reductions in energy costs and service costs for the dispensers, and increased efficiency in servicing and understanding usage of the dispensers, including planning and ordering of supplies and deployment of personnel for servicing the dispensers, as well as helping to facilitate the integration and efficiencies in operation of equipment and compliance monitoring and tracking within a facility.

For example, in some embodiments, such as generally indicated in FIG. 7D, the lead dispenser can be positioned at a selected location within an area or space and each of the drone dispensers can be arranged at various other locations within the area or space (e.g., lead dispenser can be located near a door of a bathroom and the drone dispensers can be placed at other locations such as along sinks or in stalls of the bathroom) and will communicate with at least the lead dispenser, and in embodiments, also with one or more drone dispensers within the area or space. As further discussed below, the drone dispensers can communicate with the lead dispenser to send usage and diagnostic information to and receive instructions/updates and queries from the lead dispenser. In embodiments, the lead dispenser can collect and transmit the usage and diagnostic information from multiple ones of the drone dispensers as part of one or more information packets.

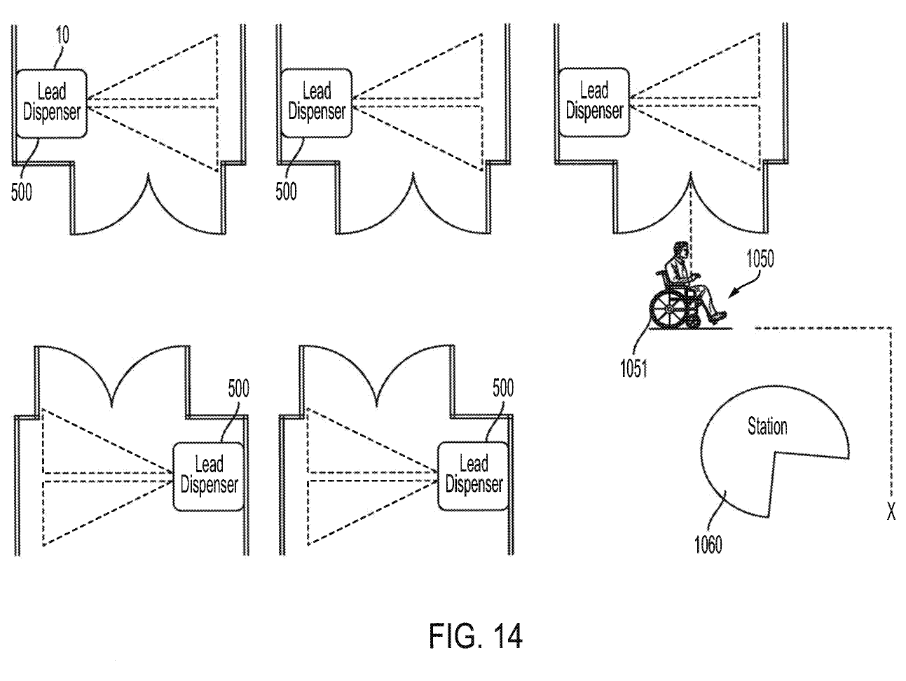

Alternatively, or in addition, in embodiments such as indicated in FIG. 14, a lead dispenser of a series of dispensers configured to act as lead dispensers can be positioned at spaced, known locations throughout a facility and can be in bidirectional or cross communication with each other and with a plurality of drone or secondary dispensers associated therewith. By way of example, in embodiments such as indicated in FIG. 14, one or more lead dispensers 520 can be positioned along a hallway of a facility (e.g., a hospital), with a plurality of drone dispensers 522 generally being located within a sub-area such as a patient room. Each room also can have a lead dispenser with a series of associated drone dispensers in communication therewith. The lead dispensers can communicate with the associated drone dispensers to collect usage and diagnostic information therefrom and can communicate with one or more other lead dispensers (e.g., a lead dispenser in the hall outside the room). Dispenser usage and diagnostic information for each dispenser in the room can be collected by the lead dispenser in the room, which can transmit this information to an overall or primary lead dispenser for collection and transmission to the facility control system, server, cloud server or other network, mobile device or software application, etc.

As illustrated in FIG. 7D, in embodiments, the lead dispenser 520 further can be in communication with a network 524, such as cloud based network or other suitable public (e.g., the Internet) or private network, and the lead dispenser 520 can provide one or more signals, packets, etc. including, or otherwise related to, dispenser information and/or alerts, notifications, etc., generated by the lead dispenser 520 and the drone dispensers 522 to the network 524 for access by a system operator, maintained personnel, etc. The dispenser information can include information related to power levels (e.g., battery levels), supply levels (e.g., information related to remaining amounts of sheet material or liquid), usage (e.g., times and dates of when the dispenser was used, amounts the dispenser was activated during a specific time period, other usage rates or statistics, etc.) The alerts, notifications, etc. can be generated, e.g., if the dispensers 520/522 are experiencing a low power, low supply, error states, etc.

The lead dispenser 520 and drone dispensers 522 may include a short-range receiver/transmitter 526A/526B, such as a Bluetooth® or other suitable RF or short-range signal receiver/transmitter, to facilitate communication between the lead dispenser 520 and the drone dispensers 522 (see, e.g., FIG. 7D). The lead dispenser 520 also can include an additional, long-range receiver/transmitter 528, such as a narrowband (“NB”) receiver/transmitter (e.g., 4G, LTE, 5G, etc.) or other suitable transmitter/receiver, e.g., Wi-Fi, for transmitting and/or receiving information to/from or otherwise communicating with the network 524.

Further, in some variations, the drone dispensers 522 can have a substantially similar construction to the lead dispenser 520 and will include a long-range receiver/transmitter 528, such as a narrowband (“NB”) receiver/transmitter (e.g., 4G, LTE, 5G, etc.) or other suitable transmitter/receiver, e.g., Wi-Fi, etc. In these variations, the long-range transmitter/receivers 528 of one or more of the drone dispensers 522 can be deactivated or generally maintained in a low power state.

As further indicated in FIG. 7D, the lead dispenser 520 and drone dispensers 520 each can include the at least one PIR sensor 512A/512B configured to detect infrared radiation of a person or people (e.g., that is indicative of the presence or movement of individuals within a prescribed detection range, area, zone, etc. covered by each of the PIR sensors 512A/512B around their respective dispensers 520/522). The PIR sensors 512A/512B can be in communication with the control system 506 and the power management system 510 of their respective dispensers 520/522. The control system 506 and dispensing assembly 501 of the respective dispensers 520/522 can be deactivated and activated based upon/responsive to signals received from their corresponding PIR sensors 512A/512B and their corresponding activation sensor 414 of the power management system 510.

When the PIR sensors 512A/512B detects infrared radiation from a person or persons (e.g., indicating an occupied state or mode—such as a person entering a bathroom or an individual stall), at least one respective activation sensor 414 for that dispenser can be connected to or otherwise placed in communication with a corresponding power source 508 so as to receive power/current therefrom. In another embodiment, when one of the respective PIR sensors 512A/512B detects infrared radiation from a person or persons, everyone or more of the dispensers 520/522 may be directed (e.g., receive a signal) to connect their corresponding activation sensor 414 to the power source 508 while the other dispenser components (e.g., the controller/control system 506, the dispensing assembly 501, and dispensing mechanism 502, various other sensors, etc.) remain disconnected from the power source 508.

For example, in embodiments, power management systems of each of the dispensers can include or can be in communication/linked with a transmitter and receiver that can send a signal to other power management systems to power-on or wake up (e.g., connect to the power supply) at least one activation sensor associated therewith. In other embodiments, the lead dispenser could be configured such that its power management system can connect its control system to power to enable the control system to send a signal to the other dispensers (e.g., drone dispensers) to place their activation sensors in a powered on or active state.

Thereafter, when the activation sensor 414 of a respective dispenser 520/522 detects infrared radiation from a person or persons, their respective control system 506, dispensing assembly 501, and other dispenser components in communication therewith can be connected to or otherwise placed in communication with their corresponding power source 508 so as to receive power/current therefrom. The other non-activated dispensers 520/522 may remain in a low power state.

When the PIR sensors 512A/512B do not detect infrared radiation from a person or people, indicating an unoccupied state or mode (e.g., no one is detected within the room or within an individual stall), their respective control system 506 and other dispenser components in communication therewith can be placed in a low or minimal power state and disconnected from the power source 508 such that no power/current is provided to the control system 506 and the dispensing assembly 501 such that the dispensing assembly 501, the activation sensor 414, and other dispenser components in communication therewith are deactivated and do not draw or consume power from the power source 508. That is, in the low or minimized power state, only the PIR sensors 512A/512B will draw or consume power from the power source 508.

In some respects, the control systems 506 (and other dispenser components in communication therewith) can finish or complete any on-going work, functions, operations, etc. thereof and generate and transmit one or more command signals to place the short-range transmitter/receivers 526A/526B (as well as the long-range transmitter/receiver 528) into a low power/power off state. As the control system 506 enters into its power down sequence, the control system 506 can generate and output one or more signals, e.g., a low level signal, to the switch 518 such that the switch 518 is in an open or “off” state, disconnecting the power source 508 from their associated dispenser controllers 506 to completely shut down/power off the dispenser controllers 506 and substantially all other components of the dispensers 520/522 including the activation sensors 414, except for the PIR sensors 512A/512B, and, in some variations, the short-range 526/526B and long-range 528 transmitter/receivers. In these variations, the transmitters/receivers 526/526B and 528 can be placed in a low power or sleep state. In this regard, the control system 506 and substantially all other components of the dispensers 520/522 (e.g., activation sensors 414, dispensing mechanisms 502, etc.) are generally decoupled from the power sources 508 such that the control systems 506 and other operative components of the dispensers 520/522 do not consume or draw power from the power sources 508 while in such low/minimal power state.

FIGS. 8A-8E show additional elements of the dispensing system 500 of FIG. 7A-7D in various states of operation. In embodiments, the dispensing system 500 in FIGS. 8A-8E may further include the dispensing mechanism 502, which may include at least one motor. In some embodiments, more than one motor circuit 602 can also be provided, including, for example, a motor one circuit 602A and a motor two circuit 602B. Two motor circuits 602A/602B may be used when the dispenser 400 includes a plurality of supplies 404 as shown in FIG. 4B.

In addition, as shown in FIGS. 8A-8E, in embodiments, the power management system 510 may be in communication with the power source 508, and a voltage regulator or limiter 604 can also be provided along the pathway between the power management system 510 and the power source. The voltage limiter also can be provided at other locations, such as between the motor circuits or other components of the dispensing assembly 501.

In some embodiments, the dispensing system 500 may further include a push button system 606, which may include a push button left 606A and a push button right 606B, the paper detection system 608 for detecting the supply level of the fluid or sheet material, which may include a paper detection left sensor 608A and a paper detection right sensor 608B depending on the type of dispenser 10/300/400/520/522 implemented, a Wi-Fi dedicated CPU 610, and a night light circuit 612. The PIR sensor 512 and the switch 518 may be included in the power management system 510 in FIGS. 8A-8E.

The dispensing system 500 is configured to selectively activate and deactivate certain elements of the dispenser based on the detected presence of a user within a prescribed area or zone around the dispenser using the PIR sensor 512 and the activation sensor 414. The elements will be further described in connection with dispensers such as previously described.

In embodiments, the power management system 510 can include a switch 518, e.g., a triode, or other suitable switching mechanism, which is coupled to a lead or other coupling/connector connecting the power source 508, and the PIR sensor 512. The control system 506, the activation sensor 414, and dispensing mechanism, and transmitter can be linked to the power management system, or the power management system can be incorporated into the control system. The switch 518 can be activated by the power management system 510 to connect and disconnect the control system 506 from the power source 508 (e.g., in FIG. 7A, the switch 518 includes a first, disconnected position wherein the control system 506 and the activation sensor 414 are disconnected from the power source 508 and the PIR sensor 512 is connected to the power source, the switch 518 includes a second, connected position wherein the activation sensor 414 is connected to the power source 508 and the control system 506 is disconnected from the power source 508, and the switch 518 includes a third, connected position wherein the control system 506 and the power source 508 are connected).

In this regard, when closed by the power management system, the switch 518 can connect or couple the controller of the control system 506 to the power source 508 such that the controller can draw or consume power from the power source 508, and when opened by the switch 518 can disconnect or decouple the controller from the power source 508 such that the control system 506 does not draw or consume power from the power source 508.

In embodiments, the switch 518 can further couple the activation sensor 414 to the power source 508 such that the activation sensor 414 can draw or consume power from the power source 508 independently of the control system and other operative components (e.g., while the controller, etc. remain disconnected from the power supply). The switch 518 also can disconnect or decouple the activation sensor 414 from the power source 508 such that at least one activation sensor 414 does not draw or consume power from the power source 508 upon a signal from the controller or other condition (e.g., expiration of a prescribed time period of no activity).

In FIG. 7C, in embodiments, there may be two switches 518, 518A and 518B. The power management system 510 may couple the activation sensor 414 to the power source 508 via the first switch 518A upon a signal from the PIR sensor 512 to the first switch 518A. Thereafter, the activation sensor 414 may couple the dispensing assembly 501 to the power source 508 via the second switch 518B once it detects a user withing a selected activation zone, by sending a signal from the activation sensor 414 to close the second switch 518B and connect the control system to the power supply.

In embodiments, the power management system 510 may operate the PIR sensor 512 to determine if the activation sensor 414 shall connect/couple or disconnect/decouple from the power source 508 via the switch 518. The power management system 510 may operate the activation sensor 414 to determine if the control system 506 shall connect/couple or disconnect/decouple from the power source 508.

Once a dispensing operation is initiated, the dispensing mechanism 502 can dispense a selected amount of fluid or sheet material when activated by the control system 506. For example, in embodiments, the dispensing mechanism 502 may include a motor 74 shown in FIG. 1B and feed roller 18 and in response to the activation signal or prompt, the controller can connect the motor to the power supply and operate the motor to drive the feed roller to feed a length of sheet material. Alternatively, in other embodiments, the dispensing mechanism, such as shown in FIG. 3, can be configured for dispensing fluids such as soap or sanitizer.

In addition, in still other embodiments, the dispenser can include dispensing mechanism 502 as shown in FIGS. 8A-8E that may include two motor circuits, a motor one circuit 602A and a motor two circuit 602B, for selectively feeding from a plurality of supplies of material. However, the dispensing mechanism 502 may only require one motor circuit depending on the type of dispenser used. The dispensing mechanism may be in communication with the power source 508 or receive power from the control system 506, e.g., the dispensing mechanism 502 may receive power from the power source 508 through the control system 506 or directly from the power source 508 as shown in FIG. 8A.

The activation sensor 414 is configured to be activated by a user to initiate dispensing of the selected amount of the fluid or sheet material. In embodiments, the activation sensor 414 may include a proximity sensor, or an infrared sensor configured to capture infrared radiation of a user within a defined activation range, area, or zone. For example, as shown in FIGS. 2 and 4A-4B, the dispenser 10 can include an activation area or zone 405 that can, in embodiments, be located above or within a recess or pocket 406 in the dispenser into which the user will place their hand for activation of a dispensing operation. In embodiments, the activation sensor 414 may be configured such that it may not detect the presence of the user to initiate a dispensing cycle or operation until the user has placed their hand inside the pocket or recess 100. In other embodiments, the activation zone can be directed along the front or a lower portion of the dispenser housing and can be focused to minimize inadvertent activations. In embodiments, the activation sensor 414 also may include a dedicated CPU for processing signals from the control system 506 and the power management system 510.

The at least one activation sensor may be powered by the power management system 510 through the switch 518 or the control system 506, depending on different embodiments of the disclosure. When the activation sensor 414 is activated and detects a user, the activation sensor 414 may send a signal to the control system 506 to activate the dispensing mechanism 502. The activation sensor 414 may operate the switch 518 to connect the control system 506 to the power source 508. In another embodiment, the activation sensor 414 may send a signal to the power management system 510 to activate the switch 518 to connect the controller of the control system 506 to the power source 508.

In embodiments where the activation zone is focused inside a pocket or recess or otherwise, the user may be generally required to take some action, such as putting their hand in the pocket or recess, to activate the activation sensor. Otherwise, the activation sensor can remain in a standby mode and will not cause the controller/main CPU of the control system to be reconnected to the power supply and signal the controller to start a dispensing cycle or operation. This can help conserve power/reduce current draw by the dispenser, by maintaining the control system, dispensing mechanism and other operative components of the dispenser in a powered off condition disconnected from then power supply, which in turn can help conserve and extend the life of batteries or a rechargeable power supply for the dispenser.

In embodiments, the control system 506 may be in communication with the activation sensor 414, the power source 508, the power management system 510, the PIR sensor 512, the switch 518, the push button system 606, the paper detection system 608, the Wi-Fi dedicated CPU 610, and the night light circuit 612. The control system 506 may control when the attached elements are activated and receive power. The control system 506 may connect to the power source 508 by the power management system 510 when the activation sensor 414 captures infrared radiation of one or more individuals within the prescribed detection range, area, or zone.

The power source 508 of the dispensing system 500 may be one or more batteries, an alternating current (AC) distribution system, or other AC or direct current (DC) power sources. The power source 508 may be in communication with the power management system 510, the control system 506, and the power management system 510.

In embodiments, the power management system 510 includes the PIR sensor 512 and the switch 518. In some embodiments, the switch 518 or other connector could be a separate element in communication with the power management system 510. The PIR sensor 512. An embodiment of PIR sensor 512 is shown in FIG. 9. The PIR sensor 512 may have a larger prescribed detection range, area, or zone than the activation sensor 414. The power management system 510 may be in communication with the control system 506, the activation sensor 414, and the switch 518. In addition, in some embodiments, the activation sensor 414 could comprise a similar infrared sensor.

In embodiments, the power management system 510 may be in communication with the paper detection system 608, the Wi-Fi dedicated CPU 610, and the night light circuit 612. The power management system 510 may operate the switch 518 to connect and disconnect the control system 506, the activation sensor 414, and other elements to the power source 508.

In embodiments, the power management system 510 may determine which elements need power based on sensor inputs from the PIR sensor 512 and the activation sensor 414. The power management system 510 further includes a processor, such as a microprocessor, CPU, etc., memory, and computer programming stored in the memory and executed by the processor for control of the switch 518 and the PIR sensor 512. In embodiments, the PIR sensor can include a processor and a memory (or can be in communication with a memory) and programming sense or detect infrared radiation and operate the switch. The power management system 510 (e.g., the CPU of the PIR sensor) also may include a timer to determine a time period lapsed during the power saving states as described below in relation to FIGS. 8B-8E.

The Wi-Fi dedicated CPU 610 can include a long-range receiver/transmitter such as a narrowband (“NB”) receiver/transmitter (e.g., 4G, LTE, 5G, etc.) or other suitable transmitter/receiver, e.g., Wi-Fi, for transmitting and/or receiving information to/from or otherwise communicating with a network. Further, in some variations, the long-range transmitter/receivers of the dispensing system 500 can be deactivated or generally maintained in a low power state. The Wi-Fi dedicated CPU 610 may transmit sensor information and the status of the fluid or sheet material supply as determined by the paper detection system 608. The Wi-Fi dedicated CPU 610 may include the short-range receiver/transmitter 526A/526B as shown, e.g., in FIG. 7D, such as a Bluetooth® or other suitable RF or short-range signal receiver/transmitter, to facilitate communication. The paper detection system 608 may include the paper detection sensor 416 in FIG. 4B to verify that the sheet material has been removed from the discharge chute.

The night light circuit 612 can illuminate at least a portion of the front recess of the dispenser assembly with visible light (e.g., proximate to and/or adjacent the dispenser activation sensor assembly 401). Accordingly, the night light circuit 612 can guide a user with respect to where the user should gesture in order to activate a dispensing operation. The night light circuit 612 can be configured to illuminate in one or more colors of light in order to provide information about the state and/or status of aspects of the dispenser assembly (e.g., supply levels, battery life, etc.). In addition, in embodiments, the night light circuit 612 can be turned on when the PIR sensor 512 detects a user and can be configured to use a minimal power draw, while one or more components of the dispensing assembly 501 can be placed in a low-power or non-active but ready state. For example, the activation sensor 414 can be connected to the power source 508 but maintained in a low power or sleep state until the user is detected by the activation sensor 414, and the other components such as the control system 506 and dispensing mechanism 502 can be maintained in a powered-off state.

Example embodiments of the power saving states of the dispensing system 500 will now be described in reference to FIG. 7A-7D and FIGS. 8B-8E.

During an unoccupied state, as shown in FIG. 7B, the PIR sensor 512 (an example of which is generally illustrated in FIG. 10), of the power management system 510 (FIG. 7B) will be in a powered on state, coupled to the power source 508 while the switch 518 is placed in an open state to disconnect from the power source 508 all other elements of the dispensing system 500, including the dispensing assembly 501, the controller/control system 506, at least one activation sensor 414, the dispensing mechanism 502 and Wi-Fi transmitter and CPU. In an embodiment, in the unoccupied state, the current draw from the power source 508 may be limited to the power necessary to activate the power management system 510 and the PIR sensor 512.

For example, in embodiments, the power management system can substantially lower the current draw by the dispensing system, such as by disconnecting or shutting the control system, dispensing mechanism and activation sensor(s) from the power supply such that those components are effectively powered down or off, and only the PIR sensor may be drawing power from the power supply. In embodiments, the current draw can be lowered to as low as about 40 µA or lower, and in some embodiments, in a range from about 10 µA to about 80 µA, about 10 µA to about 70 µA, about 10 µA to about 60 µA, about 10 µA to about 50 µA, about 10 µA to about 40 µA, about 10 µA to about 30 µA, about 10 µA to about 20 µA, about 20 µA to about 80 µA, about 20 µA to about 70 µA, about 20 µA to about 60 µA, about 20 µA to about 50 µA, about 20 µA to about 40 µA, about 20 µA to about 30 µA, about 30 µA to about 80 µA, about 30 µA to about 70 µA, about 30 µA to about 60 µA, about 30 µA to about 50 µA, about 30 µA to about 40 µA, about 40 µA to about 80 µA, about 40 µA to about 70 µA, about 40 µA to about 60 µA, about 40 µA to about 50 µA, about 50 µA to about 80 µA, about 50 µA to about 70 µA, about 50 µA to about 60 µA, about 60 µA to about 80 µA, about 60 µA to about 70 µA, and finally, about 70 µA to about 80 µA.

The control system 506 of the dispensing assembly 501, the dispensing mechanism 502, and the at least one activation sensor 414 will remain disconnected from the power source 508 when the dispensing system 600 is in the unoccupied or non-triggered state. The dispensing system 500 may remain in an unoccupied state until the PIR sensor 512 captures infrared radiation of one or more individuals within the prescribed detection range, area, or zone.

The dispensing system 500 may be in an occupied state as shown in FIG. 8C when an individual’s presence is detected by the PIR sensor 512. The occupied state may be activated when the PIR sensor 512 captures infrared radiation of one or more individuals within the prescribed detection range, area, or zone. In the occupied state, the power management system 510 may connect or couple via the switch 518 the activation sensor 414 to the power source 508. The power management system 510 may disconnect or decouple from the power source 508 the control system 506, the dispensing mechanism 502, and the paper detection system 608, and other components of the dispensing assembly 501 while the activation sensor 414 is connected to the power source 508. Therefore, the only elements drawing current are the activation sensor 414 and the power management system 510 including the PIR sensor 512. In an embodiment, in the occupied state, the dispensing system may draw an electric current of about 260 µA.

In an embodiment, the power management system 510 may connect or couple, via the switch 518A, the activation sensor 414 to the power source 508. The other operative components such as the control system, dispensing assembly, other sensors, etc. can stay disconnected from power.

In an alternative embodiment, when the dispensing system 500 is in an occupied state, the power management system 510 may connect or couple the dispensing assembly 501 to the power source 508 so as to be ready to dispense, while the controller of the control system 506 may remain in a low power or sleep mode. The sleep mode of the control system 506 draws less power than when the control system 506 is activating the dispensing mechanism 502 and is in a ready to dispense state. The control system 506 may draw enough power from the power source 508 to activate the activation sensor 414 and the night light circuit 612.

In another embodiment, when the dispensing system 500 is in an occupied or fully powered-on and operational state, the power management system 510 may connect the night light circuit 612 to illuminate the activation zone of the activation sensor 414.

In an embodiment, the dispensing system 500 may remain in the occupied state for a predetermined period of time and then return to the unoccupied state.

The dispensing system 500 may be in a ready to dispense state as shown in FIG. 8D. The ready to dispense state may be activated when the dispensing system is in the occupied state and the activation sensor 414 detects a user within the prescribed detection range, area, or zone of the activation sensor 414. When a user is detected by the activation sensor 414, the power management system 510 may connect or couple the dispensing assembly 501 including the control system 506 to the power source 508. The power management system 510 and the control system 506 may further connect to the power source 508 the paper detection system 608, the dispensing mechanism 502, the night light circuit 612, the push button system 606, and the paper detection system 608. In the ready to dispense state, the control system 506 may signal the dispensing mechanism 502 to dispense the fluid or sheet material when the activation sensor 414 detects a user within the prescribed detection range, area, or zone.

In an embodiment, the activation sensor 414 may connect or couple the dispensing assembly 501 to the power source 508 via the second switch 518B.

In an embodiment, the dispensing system 500 may remain in a ready to dispense state for a period of time for the dispensing mechanism 502 to dispense the fluid or sheet material and then the dispensing system 500 may return to the occupied state. In another embodiment, the dispensing system 500 may remain in the ready to dispense state for a predetermined period of time before returning to the occupied state.

The dispensing system 500 may be in a ready to dispense and ready to send state, ready to send a data packet including dispenser status information as shown in FIG. 8E when the control system 506 receives a signal from the push button system 606 while in the ready to dispense state. When in the ready to dispense and ready to send state, the control system 506 and the power management system 510 may activate the Wi-Fi dedicated CPU 610. The Wi-Fi dedicated CPU 610 may transmit dispenser status information or data from the paper detection system 608 to notify an administrator of the usage and/or status of the dispenser. For example, the dispenser status information can include dispenser usage and diagnostic information (e.g., activity such as a number of dispensing operations, times of peak use, who used the dispenser, fault conditions, a supply status of the liquid or sheet material within the dispensing system 500, and/or other data. The dispenser of the dispensing system 500 may remain in the ready to dispense and ready to send data packet state for a predetermined period of time before returning to the occupied state or the ready to dispense state.

In addition, in embodiments, the dispenser(s) of the dispensing system, and/or individual components such as the power management system, control system, activation system, and other components, can incorporate a smart architecture. For example, in embodiments, a dedicated CPU or processor of the PIR sensor 512 of the power management system, the main CPU or controller of the dispenser control system 506, the CPU 610 for the Wi-Fi connection and/or transmission system, the CPU for the activation/hand sensor 414 and CPUs for the one or more paper detection sensors, and a dedicated CPU or processor for the dispensing mechanism (e.g., in embodiments, each motor of the dispensing mechanism can include a dedicated CPU, or in some embodiments, the motors could be linked as part of a package or series responsive to a common CPU), can be configured as replaceable, “plug-and-play” components that can be replaced or changed as needed without substantively affecting the operation of the dispenser. This can enable the dispenser to be scalable or adapted to meet different uses and/or changing usage conditions.

In addition, with each of the operative components having its own CPU or processor/controller, each component can be selectively operated, independently of the others, which can further promote load shedding to help reduce power consumption by the dispenser. For example, as indicated in FIGS. 8A-9E, in embodiments, when the dispenser is initially set-up for operation, the PIR sensor may be the only component in operation and receiving power (e.g., FIG. 86B).

In embodiments, when the PIR detects infrared radiation, it can cause one or more other components to be activate, for example, connecting the activation sensor to the power supply and sending a signal to the CPU of the activation sensor to cause the activation sensor 414 to activate/be readied for detection of a user (e.g., detecting a user’s hand being placed near the activation/hand sensor 414). The activation sensor can be controlled by its CPU to actively or passively scan for a user, and in embodiments, can be maintained in its activated state for a selected time period, after which, if not activated, it can be powered down, or can be put in a sleep mode at a lower power if the PIR sensor detects infrared radiation within its predefined zone or area, while the main CPU of the control system, the CPU’s of the dispensing mechanism, paper detection sensors, Wi-Fi connection/transmitter, and night light circuit can remain disconnected from the power supply. Thereafter, when the activation sensor detects the presence of a user, its CPU can cause the main CPU to be connected to the power supply and can send an activation signal or prompt to the main CPU to start a dispensing cycle, at which point, the main CPU can connect the dispensing mechanism to the power supply and cause the motor(s) thereof to feed a length of sheet material or supply a selected amount of a fluid.

The other components (e.g., the paper detection sensor(s), Wi-Fi connection/transmitter, and night light circuit) each can selectively remain disconnected from the power supply or can be powered on with the reconnection or powering up of the activation sensor, the main CPU, and/or the dispensing mechanism. Alternatively, one or more of the components such as the paper detection sensor(s), Wi-Fi connection/transmitter, night light circuit, or other components can be individually connected to power and placed in an operable state, while other components such as the activation/hand sensor can remain powered-off (e.g., disconnected from the power supply). In some embodiments, some such other components (e.g., the control system) can be connected to power but can be put into a low power or sleep mode, or, in some embodiments, disconnected from power to further help minimize power consumption by the dispenser.

In various embodiments, the control system 506 of each dispenser of the dispensing system 500 also can be configured to send an alert or transmit notifications and/or data to a building control or server, or to an administrator to notify them of a fault condition such as low power, low sheet material or fluid supplies, malfunctions, usage data, etc. The control system 506 further can be programmed to send such an alert or transfer usage data, etc. once the control system 506 has been reconnected to power, generally as an automated function. By way of example, in embodiments such as indicated in FIG. 7E, once the control system 506 is connected to power, the CPU controlling the Wi-Fi connection can likewise be connected to power, either in conjunction with the control system, or upon command of the control system establishing the power connection, and once a network connection (e.g., to building personnel/management, a central server, building/facility management server, the cloud, etc..) has been established, an alert or notification can be sent automatically. After the alert or notification has been sent, the Wi-Fi CPU can be powered down or disconnected from the power supply, even if the dispenser is still in an occupied/dispensing state. In embodiments, the Wi-Fi CPU can remain in its powered on state until a confirmation of receipt of the alert or notification by a recipient (e.g., building personnel/management, a central server, building/facility management server, the cloud, etc..), and once confirmation is received, can then be powered down or off.

In other embodiments, such alerts, notifications, data transfers or other communications can be sent at selected time intervals (e.g., the power management system 510 can include programming to wake up the control system 506 after an elongated period of inactivity to communicate with a central server, such as an administrator control, etc..). Alternatively, the CPU of the Wi-Fi transmitter or communication assembly can be connected to power while the control system, etc. remain disconnected. The Wi-Fi CPU can be connected at specified times, or when the PIR detects infrared radiation and can send a data pocket transmission and then can be powered off or down. For example, as indicated in FIG. 9E, the Wi-Fi CPU can be placed in a sleep or low power mode while the dispenser is in a ready to dispense mode, and activated when an alert of notification is to be sent, and then can be put back in a low power or sleep mode.

Alternatively, or in addition to communications sent on a selected time basis, such alerts, notifications, data transfers or other communications can be sent when a problem is detected. For example, if a low supply, paper jam, low power, or other fault condition is detected when the control system is connected to power by the power management system, the Wi-Fi CPU can be transitioned to a powered on state and can send the alert or notification to the dispenser, such as indicated in FIG. 9E.

Still further, in some embodiments, the dispensing system 500, and individual dispensers 10 thereof, can be configured to download data, alerts, etc. regarding the performance of the dispenser(s)/dispensing system 500 to a storage medium such as a hosted, cloud-based storage. For example, the control system 506 of each dispenser, or a lead dispenser 520 of a system of multiple dispensers (FIG. 7D), can be programmed to automatically download data regarding the dispenser(s) performance, fault conditions, etc.., such as when powered on, or at a time interval such as specified time or times of the day. Users, such as homeowners, businesses, etc. can be provided with a storage subscription where such data can be stored and accessed as needed.

FIGS. 9A–9E show an embodiment of the dispensing system 700, which incorporates similar features and components as shown with respect to the dispensing system 500. FIGS. 9A-9E show the dispensing system in unpowered, unoccupied, occupied, ready to dispense, and Wi-Fi powered on states as determined by the power management system 510 and the control system 506.

FIGS. 9A–9E further show additional details of a power management system 510 for a dispenser, which can be in communication with at least one switch 518 for connecting various components of the dispenser to power or otherwise placing such components in a powered-on or active state. The dispensing system 700 may also include a boost IC 702 (boost converter integrated circuit) for increasing a lower input voltage to a higher output voltage. Additionally, a second voltage regulator or limiter 604B may be used to provide a stable output voltage to the control system 506.

In addition, as indicated in FIGS. 9A-9E, in some embodiments, in addition to a mina CPU or dispenser controller 506, the various operative elements of each dispenser of the dispensing system (e.g., one or more hand sensing IRMs, one or more paper detection sensors, motors, transmitter, one or more receivers) can each include a controller. Such operative components can be in bidirectional communication with each other so as to enable selective operation of such components as needed, allowing for enhanced load shedding as well as shut down of such components at least individually, or, in some cases as part of a shutdown of the entire dispenser, including potentially placing its power management system in a powered-down or powered-off state. For example, in a distributed system such as shown in FIG. 7D, one or more drone dispensers, such as those in one or more bathroom stalls, can be placed in a powered-down or powered-off state, while others, such as soap or towel dispensers in the bathroom, can remain in a powered-on state and can communicate updates re: usage to the lead dispenser.

In addition, in some embodiments, the PIR sensor of the power management system of each dispenser can be configured to selectively connect one or more dedicated controllers (e.g., a processor or CPU) configured for controlling operation of one or more different components, and/or the overall dispenser control system controller, to power or place such controllers in a powered-on state. In embodiments, each of the component controllers can be configured to communicate directly with the PIR controller and with one or more of the component controllers.

In embodiments, the activation sensor 414 (e.g., a hand sensor), in addition to its dedicated CPU, can include a hand receiving infrared motion sensor (IRM) 704, which is used to monitor movement near the dispenser when the dispenser is in the occupied state. For example, in embodiments where the dispenser can include a pocket or recess in which at least one activation sensor is positioned, the activation sensor can generate an activation zone that can be focused e.g., can be configured to have a selected detection range, within the confines of the pocket, or in a selected area along a portion of the dispenser such that a user will be required to take some affirmative action, such as placing their hand within the pocket or recess to be detected by the activation sensor.

In embodiments, the IRM can be configured to operate in similar fashion to the PIR sensor of the power management system. For example, the activation sensor can be maintained in a powered off state disconnected from the power supply, and when the PIR sensor or the power management system detects infrared radiation within the prescribed area, space or zone, it can initially activate a switch to connect the IRM to power, while, in embodiments, the CPU of the activation sensor remains in a low power mode (an in some embodiments, possibly remaining disconnected from the power supply) until the IRM detects the presence of the user and wakes the activation sensor CPU.

Alternatively, in some embodiments, the IRM sensor can remain connected to the power supply and can separately wake-up or cause the operative components of the dispenser to be connected to the power supply. For example, in embodiments, the PIR sensor can, upon detection of infrared radiation, connect the control system to the power supply, but the control system can be maintained in a low power or sleep mode. When the IRM sensor detects the user’s hand and wakes up the activation sensor and causes an activation signal or prompt to be sent to the controller, the controller and one or more operative components of the dispenser can be connected to power and/or placed in a fully powered on (occupied) and awake/ready to dispense state (FIGS. 9C-9E).

As indicated in FIGS. 9C-9E, in embodiments, when the dispenser is in its powered on (occupied) state, some of the operative components such as the motors and paper detection sensors can remain in a powered off or low power state and can be turned on when the dispenser is ready to dispense, as indicated in FIG. 9D. However, in embodiments, some features of the dispenser, such as the Wi-Fi connection can remain in a powered off condition. As noted, the Wi-Fi CPU can be maintained disconnected from the power supply, or it can be placed in a sleep or low power mode until the dispenser is placed in the ready to dispense state.

According to the present disclosure, the term “about” can be understood to cover values in the range of ±0.5 μA, though about can reflect any suitable value range, such as ±0.1 μA, ±1 μA, or up to ±3 μA, or other value ranges as will be understood by those skilled in the art. In this regard, the dispensing system according to embodiments of the present disclosure helps to save significant power in comparison to typical steady state dispensing systems. In still other still other embodiments, if, for example, an alert indicating a fault condition such as a low supply condition, has been generated, the dispenser can further be configured to connect to the network and transmit an update to indicate if/when the fault condition has been cleared.

In addition, in various embodiments, the control system 506 (FIGS. 7A-9E) of each dispenser 10 of a dispensing system 500 can include or can be in communication with a communication system including one or more transmitters and/or receivers (e.g., Wi-Fi transmitter module 610) and can include one or more receivers configured to at least periodically monitor the selected activation area or zone within a determined proximity of the dispenser to detect signals being transmitted by transmitters carried by one or more individuals and/or pieces of equipment moving about the facility.

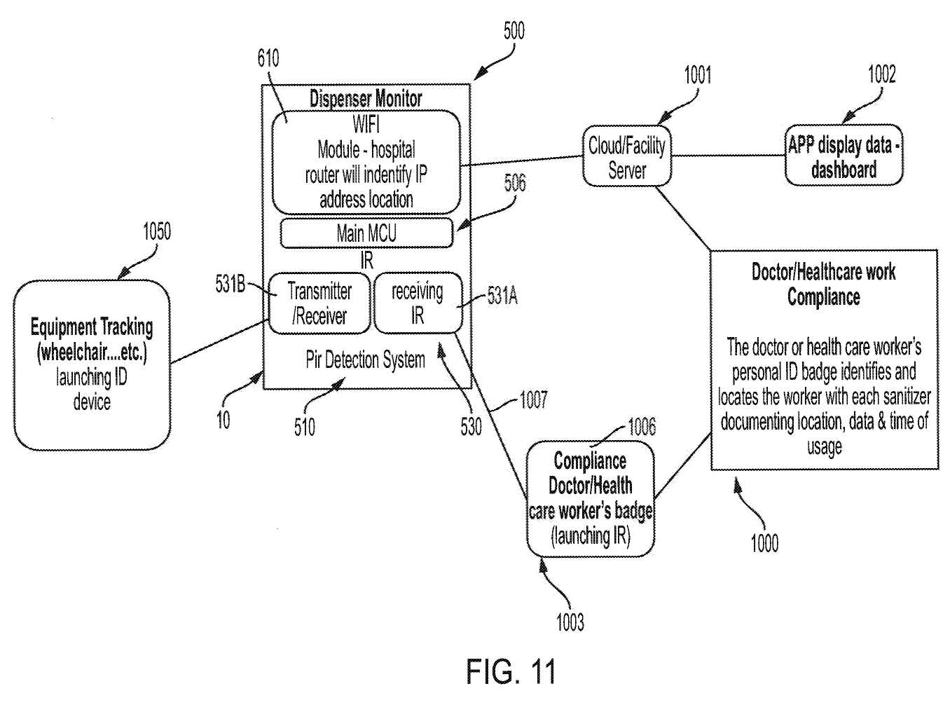

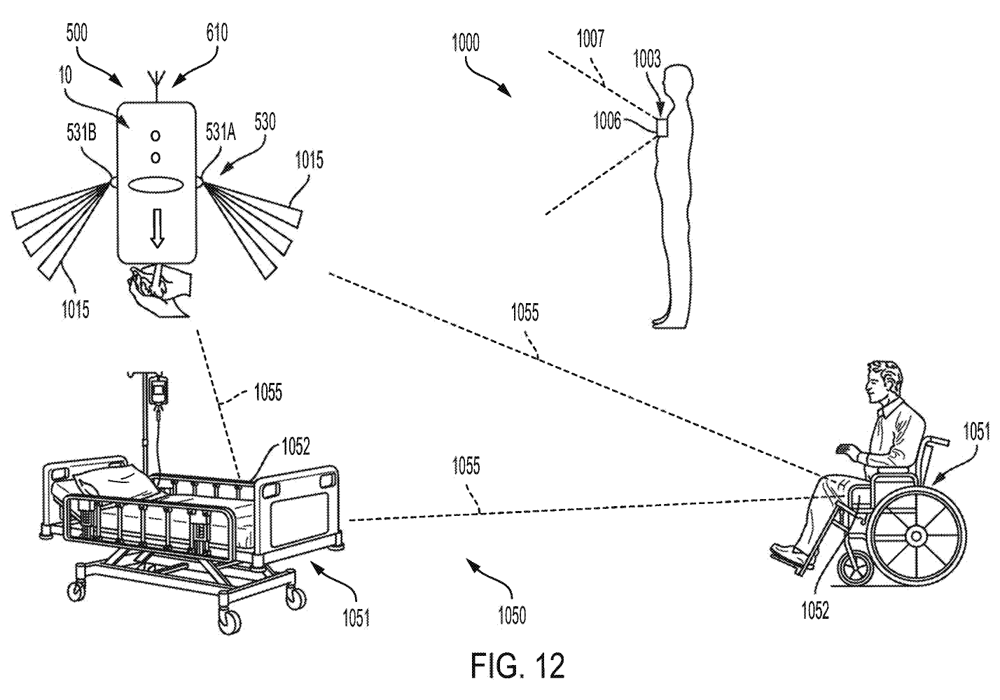

In embodiments, as schematically illustrated in FIGS. 11-12, each dispenser 10 of the dispensing system 500 further can be can be integrated or used with a compliance system 1000 for monitoring and tracking compliance with established facility procedures and protocols (e.g., health and safety protocols such as requirements for doctors, nurses and other personnel in a medical facility to perform handwashing and other sanitizing operations upon entering and/or leaving a patient’s room). In addition or alternatively, the dispensers 10 of the dispensing system 500 further can be used in conjunction with an equipment tracking system 1050 for tracking movements and facilitating location of equipment within a facility. Examples of the use/operation of embodiments of the dispensing systems as a substantially integrated part of in use in conjunction with a compliance system 1000 and/or a system 1050 for monitoring movement of and locating pieces of equipment being moved about a facility are shown in FIGS. 11-14.

In such embodiments, a system can be provided at a facility, in which equipment and/or personnel tracking systems, one or more compliance systems, and a dispensing system or plurality of dispensing systems (including one or more dispensers 10, and typically will include a plurality of dispensers) that can be linked in communication with each other and with a facility server, facility control, cloud server, network, mobile application, or combinations thereof. The tracking systems, compliance system(s), and at least a plurality of the dispensers of the dispensing system(s) within a facility can operate together as part of an integrated system; wherein the equipment and/or personnel tracking systems and compliance system(s) of the facility (e.g., a hospital or other medical facility, lab, etc.) can cooperatively communicate with a plurality of dispensers of the and dispensing system(s), which in turn can communicate with a facility control system, a server for the facility or a cloud server, or other information processing and/or storage system, for tracking and creation of records of movement of identifiable equipment and personnel about the facility and usage of dispensers by personnel, monitoring and tracking of compliance with dispenser usage protocols or procedures by such personnel, and additional dispenser usage and diagnostic and/or status information to be communicated so that the status/conditions of the dispenser, such as battery/power levels, supply levels, fault conditions and other information, can be transmitted to and accessed by the facility control system and, in embodiments, transmitted to and/or accessed by a mobile device such as a tablet, phone, etc.

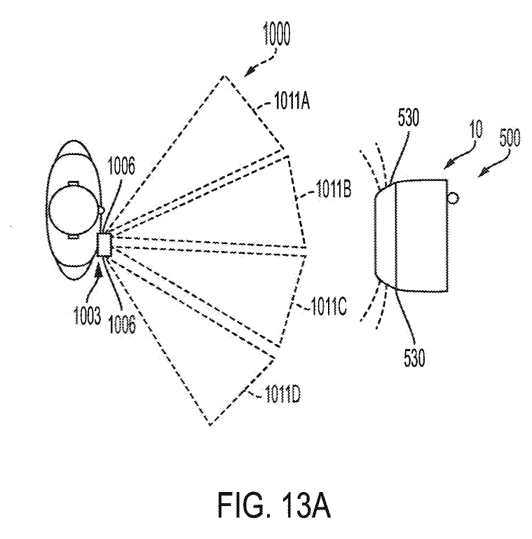

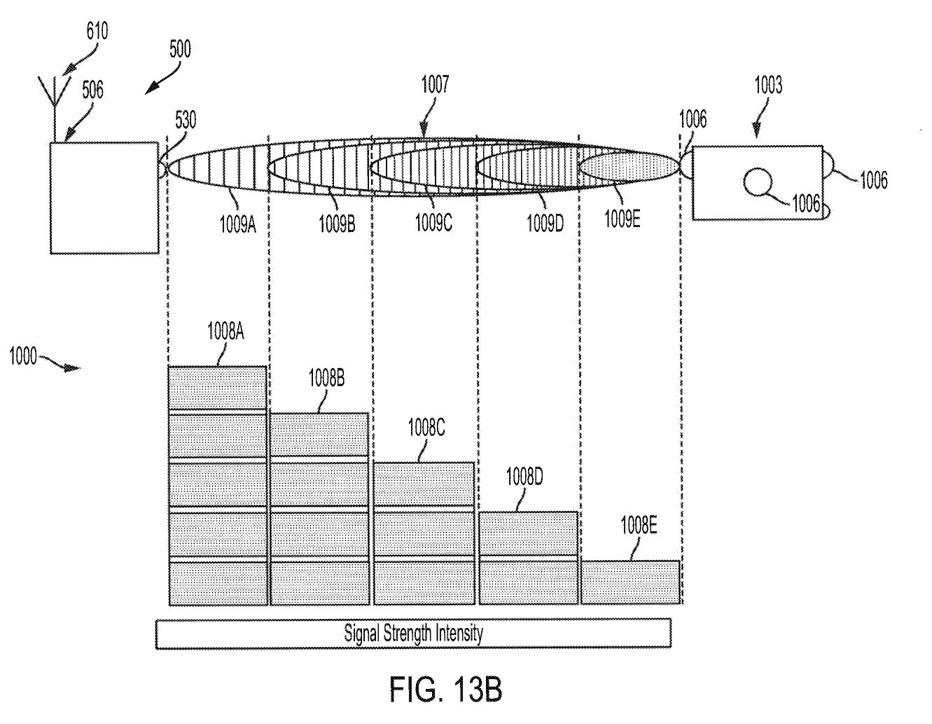

By way of example, in some embodiments, the compliance system 1000 can be linked to and/or in communication with a facility control system 1001 (e.g., a cloud/facility server) and/or can be communicated to or accessed by an application 1002, such as by a mobile or wireless device, etc., and can include a plurality of badges 1003 or other identifying devices that can be assigned to or otherwise associated with a particular individual. As generally shown in FIGS. 12-13B, in embodiments, each badge 1003 can include one or more badge transmitters 1006 that can be configured to transmit a series of signals 1007. In embodiments, each of the badge signals 1007 can include identifying a badge signature or other identifying information, such as signals with codes or other identifier, that will separately identify each badge and/or can include information identifying an individual carrying or associated with the badge. In some embodiments, the identification information within the badge signals can include an individual’s (e.g., a doctor or health care workers’) personal ID or other information.

In addition, in some embodiments, such as shown in FIG. 13B, the badge signals 1007 also can include transmission of signals having different signal strengths/intensities and/or different transmission distances/lengths and/or directions, and in some embodiments, each different signal 1008A-1008D can have its own identifier. By way of example, in some embodiments, the badges 1003 can transmit a series of badge signals 1007 in discrete bursts or signal transmissions, each of which can include at least one badge signature signal 1007 identifying the badge (and thus an individual carrying the badge), and a series of differing strength/intensity signals 1008A-1008E, different transmission distance/length signals 1009A-1009E (FIG. 13B) and/or different direction signals 1011A-1011D (FIG. 13A). The different strength signals can be used to determine a proximity to a dispenser of the person carrying the badge.

For example, if a badge carried by a doctor or nurse is initially detected and with a signal strength indicating they were outside a range that would require use of the dispenser (e.g., they were at the door of a patient’s room but did not enter), the different type/strength/distance signals detected (in addition to the badge identification signal) can be recorded at the dispenser. Thereafter, a notification can be sent with the recorded badge capture, including the identifying of an additional different type signals sent therewith, indicating whether the dispenser was used to determine if the individual was required to and did comply with facility protocols (e.g., handwashing and use of sanitizer) and if not, was it a failure of compliance or was such use not required based on the distance of the person from the dispenser when the signals were captured. Such information can also be used to track contact of individuals with patients and for mapping movements of individuals throughout the facility.

In embodiments, such as indicated in FIG. 13B, the badge transmitters 1006 can be configured to periodically send out badge signals 1007 that can be received by the control system 506 of each dispenser (e.g., by one or more receivers 530) to announce to the dispenser that a particular individual has moved within a sufficient proximity, area, range, zone or distance to the dispenser to require the individual to perform mandated health and safety protocols. In some embodiments, the badge signals 1007 can be sent substantially continuously and in response to receipt of one or more badge signals, the dispenser can be caused to be placed in a fully awake and powered-on state wherein it can communicate with the badge and also can transmit detection and/or dispenser usage and diagnostic information (which can include operational information such as a supply level, fault condition, number of dispensing operations, low power, etc.). In other embodiments, the badge signals can be sent as a string or series of signals in a repeating fashion.

In addition, in some further embodiments, the dispensers can be configured to periodically connect one or more receivers 530 thereof to power when the PIR sensor of the power management system detects infrared radiation within the prescribed detection area, zone, range, or distance. In some embodiments, the receivers 530 can include transmitters that send out signals 1015 to search for or initial communication with a badge or a tracking system 1050 (FIG. 12). The receivers further generally can have a selected area, range, zone, or distance within which they will detect a signal transmitted from a badge or tracking system.

In embodiments, the control system 506 of each dispenser of the dispensing system 500 can look for or request identification signals from an individual’s badge and multiple points/times within a dispensing operation. For example, as shown in FIG. 13B, when the control system 506 of the dispenser initially receives a first signal from the individual’s badge 1003, it can identify the presence of that particular individual and proximity to the dispenser. The dispenser control system can then search for one or more additional identification signals (e.g., at least a second signal) in conjunction with the activation of the dispensing mechanism, and search for an additional identification signal from the individual’s badge (e.g., a third signal) upon completion of the dispensing operation.

In embodiments, if, for example, one or more of the additional (e.g. second and/or third) identification signals are not received and/or a dispensing operation is not initiated after the first identification signal from the individual’s badge is detected (e.g., within a prescribed time or if some other event occurs), the dispenser control system can generate a data or information packet that can include the dispenser identification/location, a captured badge identifier, dispenser usage and diagnostic information, and a compliance/noncompliance record indicating whether the individual failed to use the dispenser.

In embodiments, such an information packet can be transmitted together with the identification of the individual associated with a detected badge to server, network, mobile application, facility control, mobile or other wireless device, or other system, for tracking and monitoring of movements of individuals throughout the facility and their compliance with health and safety protocols. In embodiments, the non-compliance record can be included as part of the dispenser usage and diagnostic information transmitted at selected times (e.g., upon powering on the control system, when the PIR sensor detects infrared radiation of one or more individuals, or at a selected time interval).

By recording the information regarding which individuals have been in proximity to a dispenser, and if such individuals were detected as present during the entirety of a dispensing operation, one or more dispensing use or operation and status records can be generated to ensure compliance with safety protocols, and also to monitor and track movements of individuals to determine if they were in areas where they should not be, or where a potentially dangerous or other condition or event occurred. The records also can be compared to a database of the facility control system (e.g., on a facility server or cloud server or network) to help in tracking and other purposes. In addition, integration of the compliance system with the dispenser enables a closer matching of an individual’s personal ID information can be matched with a location of a dispenser, and with the date and time of activation, together with a confirmation of use/non-use thereof, to help ensure true compliance with the use of the dispenser and collection of more accurate records thereof.

In additional embodiments such as shown in FIGS. 11-12, one or more systems 1050 for monitoring movement of and locating pieces of equipment 1051 within a facility can be provided and can communicate with the control system 506 of each of a plurality of dispensers located within a facility F, for example, as shown in FIG. 14. The tracking systems can be operable in conjunction with the compliance system 1000 to enable further detection, recording and tracking of equipment moving about the facility in addition to tracking and monitoring compliance of such personnel with facility protocols and procedures.

The equipment generally will comprise equipment capable of being readily moved, although larger equipment that typically may be located in a selected position or at a selected location for extended periods also can be tracking with the tracking system. By way of example, in embodiments where the facility comprises a medical facility, as shown in FIGS. 12 and 14, the pieces of equipment 1051 could include one or more of patient mobility aids, beds, monitors, portable diagnostic equipment, portable diagnostic equipment, portable treatment and life support equipment, and portable surgical equipment. In other environments, such as in a factory, equipment such as fork-lifts, dollies, etc.. can be tracked.

In embodiments, the one or more tracking systems 1050 coupled to an associated piece of equipment 1051. In embodiments, the one or more tracking systems each can include at least one tag, badge, or other similar sensor 1052 configured to detect motion of the associated piece of equipment 1051 and in response, activate a transmitter or launching device to begin transmitting tracking signals 1055 (FIGS. 11-12 and 14). For example, in some non-limiting embodiments, the at least one tag 1051 can include a switch, such as a vibration switch or similar motion sensitive detector, and further can include a controller or processor that receives a signal from the switch indicating that the piece of equipment is moving.

In embodiments, the transmitter can be configured with an adjustable launching frequency and range. For example, the frequency and/or range of the tracking signals can be set so that the tracking signals can be detected by multiple dispensers spaced throughout the facility as the equipment is moved from location to location (e.g., as a wheelchair is rolled down a hall, its tracking signals can be picked up by dispensers along the adjacent walls, and in some embodiments, by dispensers or similar equipment at other locations such as a nurses’ station 1060 (FIG. 14).

The tracking signals from the transmitter will include a signature of other identifying information for the associated piece of equipment (e.g., an item descriptor and/or other identifier such as an ID code, serial number, or the like) to allow individual pieces of equipment to be separately tracked through the hospital with the movement thereof being documented together with a location of a detecting dispenser and a date & time the tracking signals were detected by each dispenser. When the equipment stops moving, the tracking system (or at least the transmitter thereof) for that equipment is deactivated with its last location, time & date being recorded by a last dispenser receiving its tracking signals. Once the equipment is on the move again the tracking system will be activated to again transmit tracking signals identified with the piece of equipment to track its movement to a new location as required. The records of the tracking signals can be used for mapping movement of the equipment and for location thereof based on the last recorded location information.

Still further, as illustrated in FIG. 11, in some embodiments, the dispensers 10 can include at least two receivers 531A/531B configured to receive either the tracking signals or the badge signals. For example, the dispenser can have a first receiver 531A configured to receive tracking signals of a selected type (e.g., Bluetooth®, RF, infrared (IR) or other, transmissions) and/or frequency; and a second receiver 531B configured to receive the badge signals, which can include signals of a selected type such as Bluetooth®, RF, infrared (IR) or other, transmissions, but which generally will be of a different selected type and/or frequency from the tracking signals.

In addition, in embodiments, the tracking systems 1050 can be configured to communicate with other tracking systems, and/or receivers connected to other pieces of equipment. For example, in an embodiment such as shown in FIG. 12, a wheelchair can include a tracking system 1050 with a tag 1052 that sends out tracking signals 1055 that can be received by a dispenser 10, and by a receiver or tracking system coupled to another piece of equipment such as a patient bed. The tracking system of the patient bed can communicate with the tracking system of the wheelchair to identify proximity of the wheelchair to the patient bed, such as, for example, tracking whether an assigned wheelchair was used for transport of a specific patient and/or for further tracking movement of the wheelchair.

In addition, in embodiments such as generally shown in FIG. 7A, where the dispensing systems 500 include a series of dispensers 10 in communication with each other and/or a lead dispenser, at least some of the dispensers can be linked to a primary or initial power management system (e.g., for a primary or lead dispenser) that includes at least one PIR sensor. The at least one PIR sensor of such a power management system can include a CPU configured to communicate with one or more controllers of a subset of the dispensers (e.g. two or more paper towel dispensers and fluid dispensers for at least some of a series of sinks) in a selected area or location. The one or more controllers in communication with the at least one PIR sensor of such a primary of initial power management system (e.g., for a lead or primary dispenser within an area) could be a main dispenser controller configured to control substantially all operations of the dispenser, and can be placed into a powered-on or powered-up state in response to a communication from the at least one PIR sensor (e.g., the controller can be caused to turn on or be connected to power).

In other embodiments, a PIR sensor of a power management system of each dispenser can include a separate, dedicated processor or CPU configured to communicate with the at least one PIR sensor. When at least one PIR sensor of the primary power management system detects infrared radiation from an individual (e.g., as they enter a room and move near an initial or lead dispenser), it can communicate with a corresponding controller for a PIR sensor of a power management system for selected dispensers of the subset of dispensers (e.g., one or more soap and sheet material dispensers at series of sinks) to place at least one PIR sensor of such dispensers (and/or other components such as an activations sensor or the dispenser controller) into a powered-on state, connected to power, while other dispensers (e.g., sanitizer dispensers and/or tissue dispensers in a stall) can remain in a powered-off state. In addition, the remaining components of the dispensers and other dispensers (e.g., those in a bathroom stall) can be maintained in a substantially powered-off/powered-down state. At least some of the operative components of the dispensers can remain in the powered-down or powered-off state until an associated PIR sensor (which has been signaled to transition to a powered-on state to look for infrared radiation) for each of those dispensers detects infrared radiation from someone coming within a predetermined detection area, zone, range or distance of the PIR sensor.

In addition, in embodiments, such as shown in FIGS. 9A-9E, the PIR sensors can communicate with the CPU of selected components, such as an activation sensor and/or a transmitter to power those components, and one the activation sensor detects infrared radiation, it can power on the main CPU of the dispenser. Thus, in embodiments, additional reductions in power consumption via load shedding among various components of each dispenser of a dispensing system can be provided by limiting the number of active components/dispensers to those in use or likely to be used, while allowing unneeded dispensers and/or components to be shut down and placed into a substantially powered-off state.

Still further, in some embodiments, the bidirectional communication architecture of the dispensers can enable the dispensers to communicate with the tracking systems of the pieces of equipment, as well as with the badges of the compliance system(s) to place the dispensers (or at least some or the operative components thereof, in the powered-on state. For example, when a tracking signal or a badge signal is received by a corresponding receiver of a dispenser, even if the PIR sensor thereof has not detected infrared radiation, one or more operative components, such as an activation sensor or the main CPU, can be placed into the powered-on state. The dispensers further can be configured to communicate with the tracking systems and/or badges, such as by sending signals to search for responsive signals from the badges and/or tracking systems.

In embodiments, after collecting and recording such tracking signals and/or badge signals, the control system of the collecting dispenser can store the collected signal information in a memory and also can, at least periodically, establish a transmission connection (e.g., connect to Wi-Fi if not already connected) and transmit one or more information packets to a facility server, facility control, cloud server, network, mobile application, to a personal or other wireless device such as a tablet or smartphone, or combinations thereof. In embodiments, the information packets can include a location identifier for the dispenser and signature information included within the recorded tracking signal(s) for at least one piece of equipment detected moving within a prescribed range, area or zone of the dispenser. In addition, the information packets also can include signature or other identifying information for each badge detected by the dispenser together with a date and time the badge signal(s) were captured; and in some embodiments, also can include dispenser usage and diagnostic information showing use of the dispenser by an identified individual and thus compliance with a corresponding dispenser use protocol or policy. In still other embodiments, the transmitted information packets can be stored in a memory, such as in a cloud storage or server, and can be accessible via an Application or dashboard.

Any of the features of the various embodiments of the disclosure can be combined with, replaced by or otherwise configured with other features of other embodiments of the disclosure without departing from the scope of this disclosure.

The foregoing description generally illustrates and describes various embodiments of the dispensing systems, dispensers and methods of the present disclosure. It will, however, be understood by those skilled in the art that various changes and modifications can be made to the above-discussed construction of the dispensing systems, dispensers and methods of the present disclosure without departing from the spirit and scope of the dispensing systems, dispensers and methods of the present disclosure as provided herein, and that it is intended that all matter contained in the above description or shown in the accompanying drawings shall be interpreted as being illustrative, and not to be taken in a limiting sense.

Furthermore, the scope of the present disclosure shall be construed to cover various modifications, combinations, additions, alterations, etc., above and to the above-described embodiments, which shall be considered to be within the scope of the dispensing systems, dispensers and methods of the present disclosure. Accordingly, various features and characteristics of the dispensing systems, dispensers and methods of the present disclosure as discussed herein may be selectively interchanged and applied to other illustrated and non-illustrated embodiments thereof, and numerous variations, modifications, and additions further can be made thereto without departing from the spirit and scope of the dispensing systems, dispensers and methods of the present disclosure as set forth in the appended claims.

What is claimed is:

1. A system comprising:
a plurality of tracking systems, each tracking system linked to an associated piece of equipment and configured to transmit one or more tracking signals including signature information identifying the associated piece of equipment;
a plurality of dispensers positioned at selected locations within the facility, each dispenser comprising:
a dispensing mechanism configured to dispense a fluid or a sheet material;
at least one receiver configured to receive the one or more tracking signals transmitted by the tracking systems; and
a control system in communication with the dispensing mechanism and the at least one receiver and including programming configured to:
control the dispensing mechanism to dispense an amount of the fluid or the sheet material upon detection of a user initiating a dispensing operation; and
transmit an information packet including a location identifier for the dispenser and signature information for at least one piece of equipment detected moving within a prescribed range, area or zone of the dispenser to a facility server, facility control, cloud server, network, mobile application, mobile or other wireless device, or combinations thereof.

2. The system of claim 1, wherein each tracking system comprises at least one sensor configured to detect the movement of its associated piece of equipment and activate at least one transmitter to transmit the one or more tracking signals as the associated piece of equipment moves about a facility.

3. The system of claim 2, wherein each tracking system further includes at least one processor configured to receive a movement signal from the at least one sensor indicating movement of the associated piece of equipment and activate the at least one transmitter; wherein when the associated piece of equipment is determined to have stopped or be in a non-moving state, the at least one transmitter is placed in an inactive state.

4. The system of claim 1, wherein the facility comprises a medical facility, and wherein the pieces of equipment comprise one or more of patient mobility aids, beds, monitors, portable diagnostic equipment, portable diagnostic equipment, portable treatment and life support equipment, and portable surgical equipment.

5. The system of claim 1, wherein each dispenser further comprises at least one activation sensor in communication with the controller and configured to detect an activation gesture by the user and send an activation signal to the control system to initiate the dispensing operation.

6. The system of claim 5, wherein one or more of the dispensers further comprises a power management system including at least one detection sensor configured to detect a presence of one or more individuals within a prescribed detection range, area, or zone thereof, and in response, connect the control system, the at least one receiver, the at least one transmitter, the at least one activation sensor, the dispensing mechanism, or combinations thereof to a power source.

7. The system of claim 1, further comprising a compliance system comprising a plurality of badges carried by a plurality of individuals, each badge being configured to transmit a series of signals including information identifying each badge; wherein the signals are transmitted for a distance, with a selected signal strength, or combinations thereof, to be received by a dispenser when the badge moves within a selected zone, area, proximity or range of the dispenser.

8. The system of claim 7, wherein the information packet transmitted by the at least one transmitter of each dispenser further includes information each badge detected within the selected zone, area, proximity, or range of the dispenser during a prescribed period.

9. A method of tracking and/or locating pieces of equipment within a facility, comprising:
detecting movement of a piece of equipment by a tracking system linked to the piece of equipment;
upon detection of the movement of the piece of equipment, emitting a plurality of tracking signals, the tracking signals including signature information identifying a piece of equipment linked to the tracking system; and
as the piece of equipment moves about the facility, detecting at least one tracking signal emitted by the tracking system at each of a plurality of dispensers; and
at least periodically transmitting one or more information packets including a location identifier for each dispenser and one or more recorded tracking signals associated with one or more pieces of equipment from each dispenser of the plurality of dispensers.

10. The method of claim 9, further comprising:
detecting one or more signals emitted by badges carried by individuals moving about the facility at one or more dispensers of the plurality dispensers; wherein the one or more signals emitted by each badge include information identifying the badge; and upon detection of at least one signal emitted by at least one badge at a dispenser, determining usage of the dispenser by an individual carrying the at least one badge.

11. The method of claim 10, wherein determining usage of the dispenser comprises activating the dispenser to initiate a dispensing operation for dispensing a fluid or sheet material in response to an activation prompt; receiving one or more additional signals from the at least one badge by the dispenser; and determining if the individual carrying the at least one badge used the dispenser sufficient to comply with a protocol for the facility.

12. The method of claim 9, wherein:
each dispenser includes a controller, a dispensing mechanism, at least one activation sensor, one or more receivers configured to receive the signals transmitted by the badges, and a power management system including at least one PIR sensor; and
further comprising:
detecting infrared radiation of one or more individuals within a prescribed detection range, area, or zone of the dispenser with the at least one PIR sensor;
placing the at least one activation sensor in a powered-on state;
if the at least one activation sensor detects an individual moving within a selected activation zone of at least one dispenser of a dispensing system:
placing the controller of the dispenser in a powered-on state,
transmitting an activation signal to the controller; and
initiating a dispensing operation to dispense an amount of fluid or sheet material.

13. The method of claim 9, further comprising mapping movement of the piece of equipment within the facility based on a location of each dispenser at which the piece of equipment was detected; and determining a location of the piece of equipment based on a last dispenser location at which the piece of equipment was detected.

14. The method of claim 13, further comprising discontinuing transmission of the tracking signals when movement of the piece of equipment is not detected by the tracking system linked thereto.

15. A facility monitoring system comprising:
a plurality of dispensers positioned throughout the facility, each of the dispensers comprising:
a dispensing mechanism operable for dispensing a fluid or sheet material; a control system including a controller in communication with the dispensing mechanism, at least one activation sensor, and at least one transmitter;
a plurality of tracking systems each linked to an associated piece or equipment and each configured to transmit one or more tracking signals;
a series of badges configured to be carried by individuals moving about the facility, each of the badges including one or more transmitters configured transmit a series of signals including information identifying each badge;
wherein as the pieces of equipment and the individuals carrying the badges move about the facility, the tracking signals and the signals from the badges are received by one or more of the dispensers.

16. The facility monitoring system of claim 15, wherein at least some of the dispensers further comprise a power management system including the at least one PIR sensor configured detect infrared radiation or at least one radar sensor configured to detect movement; wherein the at least one PIR sensor or at least one radar sensor are configured to connect one or more of the at least one activation sensor, the controller, the transmitter, the dispensing mechanism, or combinations thereof, to power when the at least one PIR sensor captures infrared radiation of one or more individuals within a prescribed detection range, area, or zone, or the at least one radar sensor detects movement of the one or more individuals within a prescribed detection range, area, or zone; and wherein upon connection to power, the at least one transmitter is configured to at least periodically transmit the dispenser status information.

17. The facility monitoring system of claim 15, wherein each of the dispensers further comprises at least one receiver configured to receive the tracking signals transmitted by the tracking systems, and at least one receiver configured to receive the signals transmitted by the badges.

18. The facility monitoring system of claim 15, wherein the controller of each dispenser includes programming configured to transmit one or more information packets including information identifying a location of the dispenser, and including one or more tracking signals detected by the dispenser, information identifying one or more badges detected by the dispenser, dispenser status information, or combinations thereof.

19. The facility monitoring system of claim 15, wherein each tracking system comprises a vibration detector configured to detect movement of the piece of equipment and a transmitter configured to transmit the one or more tracking signals in response to detection of the movement of the piece of equipment is detected.

20. The facility monitoring system of claim 15, wherein the control system of each dispenser is configured to monitor and receive a series of signals from at least one badge within a threshold range, zone, area or proximity to the dispenser and determine whether a dispensing operation was initiated while the series of signals were detected, and if a dispensing operation is initiated, monitor and receive signals from the at least one badge at multiple times during a dispensing operation to determine usage of the dispenser by an individual carrying the at least one badge.
